(19) United States
(12) Patent Application Publication
(10) Pub. No.: US 2004/0121307 A1
(43) Pub. Date: Jun. 24, 2004

(54) Early detection of cellular differentiation using optophoresis

(75) Inventors: Catherine A. Schnabel, La Jolla, CA (US); Ilona A. Kariv, Carlsbad, CA (US); Thomas D.Y. Chung, Carlsbad, CA (US)

Correspondence Address: O'MELVENY & MEYERS, 114 PACIFICA, SUITE 100, IRVINE, CA, 92618, US

(73) Assignee: Genoptix, Inc

(21) Appl. No.: 10/326,905

(22) Filed: Dec. 19, 2002

(51) Int. Cl. C12Q 1/00

(52) U.S. Cl. 435/4

(57) ABSTRACT

A method for detecting cellular differentiation using Optophoretic analysis is provided. The method includes the steps of providing a plurality of cells, moving an optical gradient relative to the plurality of cells so as to cause displacement of at least some of the plurality of cells, measuring the travel distance of at least some of the plurality of cells, repeating the steps of moving the optical gradient and measuring the travel distance of at least some of the plurality of cells, and identifying those cells having changing travel distances. The method is able to detect the early onset of cellular differentiation. The method is also applicable to detecting and monitoring adipogenesis.

### RELATED APPLICATIONS

This Application is related to U.S. application Ser. No. ______, entitled “Detection and Evaluation of Chemically-Mediated and Ligand-Mediated T-Cell Activation Using Optophoretic Analysis”, filed on Dec. 19, 2002, U.S. application Ser. No. ______, entitled “Detection and Evaluation of Cancer Cells Using Optophoretic Analysis”, filed on Dec. 19, 2002, U.S. application Ser. No. ______, entitled “Early Detection of Apoptotic Events and Apoptosis Using Optophoretic Analysis”, filed on Dec. 19, 2002, U.S. application Ser. No. ______, entitled “Quantitative Determination of Protein Kinase C Activation Using Optophoretic Analysis”, filed on Dec. 19, 2002, U.S. application Ser. No. ______, entitled “Optophoretic Detection of Drugs Exhibiting Inhibitory Effect on Bcr-Abl Positive Tumor Cells”, filed on Dec. 19, 2002, and U.S. application Ser. No. ______, entitled “Detection and Evaluation of Topoisomerase Inhibitors Using Optophoretic Analysis”, filed on Dec. 19, 2002. The above-identified related U.S. patent applications are incorporated by reference as if set forth fully herein.

### FIELD OF THE INVENTION

The field of the invention relates generally to optical interrogation methods used to determine a biological property of a cell, a population of cells, and/or cellular components. The methods preferably can be used to select, identify, characterize, and sort individual cells or groups of cells according to the biological property of interest. The methods can be used in a variety of applications including, for example, drug screening applications, toxicity applications, protein expression applications, rapid clonal selection applications, biopharmaceutical monitoring applications, quality control application, biopharmaceutical enrichment applications, viral detection, bacterial drug sensitivity screening, and environmental testing applications.

### BACKGROUND OF THE INVENTION

In the field of biology, there often is a need to discriminate and sort cells or groups of cells based on a particular biological property of interest. For example, the discrimination and separation of cells has numerous applications in pharmaceutical drug discovery, medicine, and biotechnology. As just one example, when cells are used to produce a new protein or biopharmaceutical compound, it is desirable to select those cells or groups of cells that have the highest yields. Historically, sorting technologies have utilized some affinity interaction, such as receptor-ligand interactions or reactions with immunologic targets. Sorting technologies using affinity interaction, however, often are labor intensive, costly, require tags or labels, and change the nature or state of the cells.

While biological applications are of particular interest to discriminate and sort cells, similar methods and techniques can be employed in other applications ranging from industrial applications to environmental applications.

Attempts have been made to sort and characterize particles, including cells, based on the electromagnetic response properties of materials. For example, dielectrophoretic separators utilize non-uniform DC or AC electric fields for separation of particles. See, e.g., U.S. Pat. No. 5,814,200, Pethig et al., entitled “Apparatus for Separating By Dielectrophoresis∞. The application of dielectrophoresis to cell sorting has been attempted. In Becker (with Gascoyne) et al., PNAS USA, Vol. 92, pp. 860-864, January 1995, Cell Biology, in the article entitled “Separation of Human Breast Cancer Cells from Blood by Differential Dielectric Affinity”, the authors reported that the dielectric properties of diseased cells differed sufficiently to enable separation of the cancer cells from normal blood cells. The system balanced hydrodynamic and dielectrophoretic forces acting on cells within a dielectric affinity column containing a microelectrode array. More sophisticated separation systems have been implemented. Yet others have attempted to use electrostatic forces for separation of particles. See, e.g., Judy et al., U.S. Pat. No. 4,440,638, entitled “Surface Field-Effect Device for Manipulation of Charged Species”, and Washizu “Electrostatic Manipulation of Biological Objects”, Journal of Electrostatics, Vol. 25, No. 1, June 1990, pp. 109-103. Yet others have utilized various microfluidic systems to move and sort particles. See, e.g., Ramsey, U.S. Pat. No. 6,033,546, entitiled “Aparatus and Method For Performing Microfluidic Manipulations For Chemical Analysis and Synthesis.”

Still others in the field have used light to sort and trap particles. One of the earliest workers in the field was Arthur Ashkin at Bell Laboratories, who used a laser for manipulating transparent, μm-size latex beads. Ashkin's U.S. Pat. No. 3,808,550 entitled “Apparatuses for Trapping and Accelerating Neutral Particles” disclosed systems for trapping or containing particles through radiation pressure. Lasers generating coherent optical radiation were the preferred source of optical pressure. The use of optical radiation to trap small particles grew within the Ashkin Bell Labs group to the point that ultimately the Nobel Prize was awarded to researchers from that lab, including Steven Chu. See, e.g., Chu, S., “Laser Trapping of Neutral Particles”, Sci. Am., p. 71 (February 1992), Chu, S., “Laser Manipulation of Atoms and Particles”, Science 253, pp. 861-866 (1991).

Generally, the interaction of a focused beam of light with dielectric particles or matter falls into the broad categories of a gradient force and a scattering force. The gradient force tends to pull materials with higher relative dielectric constants toward the areas of highest intensity in the focused beam of light. The scattering force is the result of momentum transfer from the beam of light to the material, and is generally in the same direction as the beam. The use of light to trap particles is also sometimes referred to as an optical tweezer arrangement. Generally, utilizing the Rayleigh approximation, the force of trapping is given by the following equation:

$${F}_{g} = 2 π \cdot {r}^{3} \frac{\sqrt{{ϵ}_{B}}}{c} ( \frac{ϵ - {ϵ}_{B}}{ϵ + 2 {ϵ}_{B}} ) ( ∇ \cdot I )$$

where F_{g} is the optical gradient force on the particle in the direction toward the higher intensity, r is the radius of the particle, ε_{B} is the dielectric constant of the background medium, ε is the dielectric constant of the particle, I is the light intensity in watts per square centimeter and ∇ is the spatial derivative. FIG. 1 shows a drawing of a particle in an optical tweezer. The optical tweezer consists of a highly focused beam directed to the particle.

As shown in FIG. 1, the focused beam 12 first converges on the particle 10 and then diverges. The intensity pattern 14 relates to the cross-section of the intensity of the beam in the horizontal dimension, and the intensity pattern 16 is the cross-section of intensity in the vertical dimension. As can be seen from the equation, the trapping force is a function of the gradient of the intensity of the light. Thus, the force is greater where the light intensity changes most rapidly, and contrarily, is at a minimum where the light intensity is uniform.

Early stable optical traps levitated particles with a vertical laser beam, balancing the upward scattering force against the downward gravitational force. The gradient force of the light served to keep the particle on the optical axis. See, e.g., Ashkin, “Optical Levitation by Radiation Pressure”, Appl. Phys. Lett., 19(6), pp. 283-285 (1971). In 1986, Ashkin disclosed a trap based upon a highly focused laser beam, as opposed to light propagating along an axis. The highly focused beam results in a small point in space having an extremely high intensity. The extreme focusing causes a large gradient force to pull the dielectric particle toward that point. Under certain conditions, the gradient force overcomes the scattering force, which would otherwise push the particle in the direction of the light out of the focal point. Typically, to realize such a high level of focusing, the laser beam is directed through a high numerical aperture microscope objective. This arrangement serves to enhance the relative contribution from the high numerical aperture illumination but decreases the effect of the scattering force.

Optical trapping methods have been employed to manipulate biological materials. In 1987, Ashkin reported an experimental demonstration of optical trapping and manipulation of biological materials with a single beam gradient force optical trap system. Ashkin, et al., “Optical Trapping and Manipulation of Viruses and Bacteria”, Science, 20 Mar., 1987, Vol. 235, No. 4795, pp. 1517-1520. In U.S. Pat. No. 4,893,886, Ashkin et al., entitled “Non-Destructive Optical Trap for Biological Particles and Method of Doing Same”, reported successful trapping of biological particles in a single beam gradient force optical trap utilizing an infrared light source. The use of an infrared laser emitting coherent light in substantially infrared range of wavelengths, there stated to be 0.8 μm to 1.8 μm, was said to permit the biological materials to exhibit normal motility in continued reproductivity even after trapping for several life cycles in a laser power of 160 mW. The term “opticution” has become known in the art to refer to optic radiation killing biological materials.

The use of light to investigate biological materials has been utilized by a number of researchers. Internal cell manipulation in plant cells has been demonstrated. Ashkin, et al., PNAS USA, Vol. 86, 7914-7918 (1989). See also, the summary article by Ashkin, A., “Optical Trapping and Manipulation of Neutral Particles Using Lasers”, PNAS USA, Vol. 94, pp. 4853-4860, May 1997, Physics. Various mechanical and force measurements have been made including the measurement of torsional compliance of bacterial flagella by twisting a bacterium about a tethered flagellum. Block, S., et al., Nature (London), 338, pp. 514-518 (1989). Micromanipulation of particles has been demonstrated. For example, the use of optical tweezers in combination with a microbeam technique of pulsed laser cutting, sometimes also referred to as laser scissors or scalpel, for cutting moving cells and organelles was demonstrated. Seeger, et al., Cytometry, 12, pp. 497-504 (1991). Optical tweezers and scissors have been used in all-optical in vitro fertilization. Tadir, Y., Human Reproduction, 6, pp. 1011-1016 (1991). Various techniques have included the use of “handles” wherein a structure is attached to a biological material to aid in the trapping. See, e.g., Block, Nature (London), 348, pp. 348-352 (1990).

Various measurements have been made of biological systems utilizing optical trapping and interferometric position monitoring with subnanometer resolution. Svoboda, Nature (London), 365, pp. 721-727 (1993). Yet others have proposed feedback based systems in which a tweezer trap is utilized. Molloy, et al., Biophys. J., 68, pp. 2985-3055 (1995).

A number of workers have sought to distort or stretch biological materials. Ashkin in Nature (London), 330 pp. 769-771 (1987), utilized optical tweezers to distort the shape of red blood cells. Multiple optical tweezers have been utilized to form an assay to measure the shape recovery time of red blood cells. Bronkhorst, Biophys. J., 69, pp. 1666-1673 (1995). Kas, et al., has proposed an “optical stretcher” in U.S. Pat. No. 6,067,859 which suggests the use of a tunable laser to trap and deform cells between two counter-propagating beams generated by a laser. The system is utilized to detect single malignant cancer cells. Yet another assay proposed colliding two cells or particles under controlled conditions, termed the OPTCOL for optical collision. See, e.g., Mammer, Chem & Biol., 3, pp. 757,763 (1996).

Yet others have proposed utilizing optical forces to measure a property of an object. See, e.g., Guanming, Lai et al., “Determination of Spring Constant of Laser-Trapped Particle by Self-Mining Interferometry”, Proc. of SPIE, 3921, pp. 197-204 (2000). Yet others have utilized the optical trapping force balanced against a fluidic drag force as a method to calibrate the force of an optical trap. These systems utilize the high degree of dependence on the drag force, particularly Stokes drag force.

Yet others have utilized light intensity patterns for positioning materials. In U.S. Pat. No. 5,245,466, Burns et al., entitled “Optical Matter”, arrays of extended crystalline and non-crystalline structures are created using light beams coupled to microscopic polarizable matter. The polarizable matter adopts the pattern of an applied, patterned light intensity distribution. See also, “Matter Rides on Ripples of Lights”, reporting on the Burns work in New Scientist, 18 Nov., 1989, No. 1691. Yet others have proposed methods for depositing atoms on a substrate utilizing a standing wave optical pattern. The system may be utilized to produce an array of structures by translating the standing wave pattern. See, Celotta et al., U.S. Pat. No. 5,360,764, entitled “Method of Fabricating Laser Controlled Nanolithography”.

Yet others have attempted to cause motion of particles by utilizing light. With a technique termed by its authors as “photophoresis”, Brian Space, et al., utilized a polarized beam to induce rotary motion in molecules to induce translation of the molecules, the desired goal being to form a concentration gradient of the molecules. The technique preferably utilizes propeller shaped molecules, such that the induced rotary motion of the molecules results in translation

Various efforts have been described relating to cellular response. By way of example, Ransom et al. U.S. Pat. No 6,280,967 entitled “Cell Flow Apparatus and Method for Real-Time (Sic.) of Cellular Responses” describes an apparatus and method for the real-time measurement of a cellular response of a test compound or series of test compounds on a flowing suspension of cells. The cells and test compound or compounds are combined and then flowed through a detection zone. Typically, a label is detected indicating the response. Libraries of compounds are described. As stated, generally the detectable event requires a label.

In Zborowski et al. U.S. Pat. No. 5,974,901, entitled “Method for Determining Particle Characteristics”, and U.S. Pat. No. 6,082,205, entitled “System and Device For Determining Particle Characteristics”, methods and apparatus are described for determining at least one of a plurality of particle physical characteristics. Particularly, the particle characteristics may include particle size, shape, magnetic susceptibility, magnetic label density, charge separation, dielectric constant, and derivatives thereof. In one aspect, a uniform force field, such as a constant, uniform magnetic force field is generated, the particle is subject to that constant force field, and the velocity determined by observing the particle at multiple locations. Variations are described, such as for determining the position of the particle, though the force field is typically described as being constant. In another aspect, a pre-determined force field magnitude and direction is applied to a particle and multiple digital images are analyzed with specified other components to characterize the particles.

Various researchers have attempted to combine microfabricated devices with optical systems. In “A Microfabricated Device for Sizing and Sorting DNA Molecules”, Chou, et al., PNAS USA, Vol. 96, pp. 11-13, January 1999, Applied Physical Sciences, Biophysics, a microfabricated device is described for sizing and sorting microscopic objects based upon a measurement of fluorescent properties. The paper describes a system for determining the length of DNA by measuring the fluorescent properties, including the amount of intercalated fluorescent dye within the DNA. In “A Microfabricated Fluorescence-Activated Cells Sorter”, Nature Biotechnology, Vol. 17, November 1999, pp. 1109-1111, a “T” microfabricated structure was used for cell sorting. The system utilized a detection window upstream of the “T” intersection and based upon the detected property, would sort particles within the system A forward sorting system switched fluid flow based upon a detected event. In a reverse sorting mode, the fluid flow was set to route all particles to a waste collection, but upon detection of a collectible event, reversed the fluid flow until the particle was detected a second time, after which the particle was collected. Certain of these systems are described in Quake et al., PCT Publication WO 99/61888, entitled “Microfabricated Cell Sorter”.

Yet others have attempted to characterize biological systems based upon measuring various properties, including electromagnetic radiation related properties. Various efforts to explore dielectric properties of materials, especially biological materials, in the microwave range have been made. See, e.g., Larson et al., U.S. Pat. No. 4,247,815, entitled “Method and Apparatus for Physiologic Facsimile Imaging of Biologic Targets Based on Complex Permittivity Measurements Using Remote Microwave Interrogation”, and PCT Publication WO 99/39190, named inventor Hefti, entitled “Method and Apparatus for Detecting Molecular Binding Events”.

### SUMMARY OF THE INVENTION

In a first aspect of the invention, a method is provided for detecting cellular differentiation using an optical gradient including the steps of providing a plurality of cells, moving the optical gradient relative to the plurality of cells so as to cause displacement of at least some of the plurality of cells, measuring the travel distance of at least some of the plurality of cells, repeating the steps of moving the optical gradient relative to the plurality of cells and measuring the travel distances of at least some of the plurality of cells, and identifying those cells having changing travel distances.

In another aspect of the invention, a method of detecting adipogenesis using an optical gradient includes the steps of providing a plurality of preadipocytes, moving the optical gradient relative to the plurality of preadipocytes so as to cause displacement of at least some of the plurality of preadipocytes, measuring the travel distance of at least some of the preadipocytes, repeating the steps of moving the optical gradient relative to the plurality of preadipocytes and measuring the travel distances of at least some of the plurality of preadipocytes, and identifying those preadipocytes having increased travel distances.

In another aspect of the invention, a method of monitoring adipogenesis using an optical gradient comprises the steps of (a) providing a plurality of cells comprising preadipocytes, (b) moving the optical gradient relative to the plurality of cells so as to cause displacement of at least some of the plurality of cells, (c) measuring the travel distance of at least some of the plurality of cells, (d) repeating steps (b) and (c) a plurality of times, and (e) monitoring those cells exhibiting increased travel distances over time.

It is an object of the invention to provide a method for detecting cellular differentiation using a moving optical gradient. It is a related object of the invention to provide a method of detecting and monitoring adipogenesis using a moving optical gradient. Other objects of the invention will appear in more detail below.

### BRIEF DESCRIPTION OF THE DRAWINGS

FIG. 1 is a graphical depiction of optical intensity patterns for a prior art optical tweezer system, showing both the focus beam, a particle and the cross-section of intensity of the beam.

FIG. 2 is a cross-sectional drawing of the optical system for interfering two beams utilizing a variable path length by moving a mirror.

FIG. 3 is a schematic diagram of a system utilizing interference between two beams where the path length is varied utilizing a phase modulator.

FIG. 4 is a cross-sectional drawing of an optical system utilizing an interferometer where the path length is adjustable via a phase modulator.

FIG. 5 is a cross-sectional drawing of an optical system including an interferometer and a phase modulator for changing the optical path length, and includes a photograph of a wave pattern generated by the system.

FIG. 6 is a cross-sectional drawing of an optical system utilizing separate illumination and imaging systems.

FIG. 7 is a depiction of an optical system interfacing with a fluidic system.

FIG. 8 is a cross-sectional drawing of an optical system utilizing a moving scanning system.

FIGS. 9A and 9B are cross-sectional drawings of an optical system including a mask based generation of intensity pattern.

FIG. 10 is a side view of an array of illumination sources, illuminating a substrate or support.

FIGS. 11A, 11B and 11C show graphs of intensity, forces and potential energy, respectively, as a function of position in one exemplary embodiment of the invention.

FIG. 12A shows two particles at first positions and a superimposed optical pattern.

FIG. 12B shows the particles at second positions after illumination by the optical pattern.

FIG. 12C shows the trapping of particle B in an optical trap.

FIGS. 13A, 13B and 13C show graphs of the potential energy as a function of distance for the technique for separating particles.

FIG. 14 is a schematic drawing of a system for determining the dielectric constant of particles in various fluidic media of varying dielectric constant.

FIG. 15 is a cross-sectional drawing of particles and a light intensity profile for separating particles in a dielectric medium.

FIG. 16 shows a plan view of a microfluidic system for sorting particles or cells by means of a static optical gradient.

FIG. 17 shows a Before, After and Difference photograph of particles subject to a moving optical gradient field.

FIG. 18 is a graph of percent of cells measured in an experiment versus escape velocity, for a variety of cell types.

FIG. 19 shows photographs of sorting of two cell types in a microchannel device. Slide 1 (upper left) shows a red blood cell and a white blood cell successively entering the moving optical gradient field. Slide 2 (upper right) shows that white blood cell has been translated down by the action of the moving optical gradient field while the red blood cell has escaped translation. Slides 3 (lower left) and 4 (upper right) show that the red blood cell and white blood cell continue to flow into separate channels, completing the sorting.

FIG. 20 shows a microfluidic Optophoresis device used to sort wild type and mutant yeast strains.

FIGS. 21A, B and C show the steps in a scanning method comprising a first scanning of a particle population in phase one (FIG. 21A), a movement of illumination relative to the aligned particle population in phase two (FIG. 21B), and separation of particles in phase three (FIG. 21C).

FIG. 22 shows a sequence of graphs of light intensity and particle position for the technique shown in FIGS. 21A, B and C.

FIG. 23 A shows a cross-sectional view of components for use in a line scanning system, and FIG. 23B shows a top view of the operational space.

FIG. 24 A shows a cross-sectional view of a difffractive optical set up to generate one or more lines of illumination. FIG. 24B shows a top view of the arrangement in FIG. 24A. FIG. 24C shows a scanning mirror arrangement to generate one or more lines of illumination. FIG. 24D shows a top view of the illumination space.

FIG. 25 shows a top view of a sectioned, sample field.

FIG. 26 shows a top view of a sample field having multiple lines of illumination.

FIGS. 27A, B and C are images of the effective separation of white blood cells and red blood cells, corresponding to the phases shown in FIGS. 21A, B and C.

FIG. 28A shows a schematic representation of a bioreactor.

FIG. 28B shows a schematic representation of a bioreactor incorporating an Optophoretic cell enrichment system.

FIGS. 29A and 29B show optophoretic interrogation of a group of cells using a line scan.

FIGS. 30A and 30B show optophoretic interrogation of a group of cells using a fast scan.

FIG. 31 shows the distribution of escape velocities for U937 cells that were treated with 0.01 μg/ml PMA at 6 and 9 hours-post treatment in addition to control cells (non-treated) at the same time intervals.

FIG. 32 shows the time and dose dependence of escape velocity of U937 cells treated with PMA.

FIG. 33 shows the distribution of cells as a function of escape velocity for U937 cells treated with PMA and Bisindoylmaleimide.

FIG. 34 shows the distribution of cells as a function of escape velocity for U937 cells treated with 40 ng/ml of camptothecin after a period of 4 and 6 hours.

FIG. 35 shows the distribution of cells in various escape velocity ranges for the control, 500 ng/ml TNF, 250 ng/ml TNF, and 100 ng/ml TNF Jurkat treated cells at 48 hours.

FIG. 36 shows the effect of two TNF inhibitors, Leflunomide and Silymarin, on escape velocity. The two TNF inhibitors were used in conjunction with TNF.

FIG. 37 shows the distribution of escape velocities of U937 cells treated with 5 mM and 20 mM salicylic acid for 5 and 24 hours, respectively.

FIG. 38 shows the time course variation in escape velocity for K562 cells treated with varying concentrations of paciltaxel.

FIG. 39 shows the distribution of cells vs. escape velocity for K562 cells that were treated with 10 nM of paciltaxel at 17 and 23 hours.

FIG. 40 illustrates the measured escape velocities (average) for BV-173, EM-3, K-562, and U-937 cells treated with Gleevec.

FIG. 41 illustrates the measured (mean) travel distances for the control as well as the Gleevec-treated cells (BV-173, EM-3, K-562, and U-937) after 48 hours.

FIG. 42 shows the mean travel distance for the four treated groups of cells (BV-173, EM-3, K-562, and U-937) as well as the control.

FIG. 43 shows a histogram of the travel distance for the four treated groups of cells (BV-173, EM-3, K-562, and U-937) as well as the control.

FIG. 44 illustrates the mean travel distance for treated and untreated EM-3 cells. For the treated cells, different concentrations of Gleevec were used.

FIG. 45 shows the distribution of cells as a function of travel distance for the different concentrations of Gleevec.

FIG. 46 shows the distribution of cells as a function of escape velocity for both the control and Chang liver cells treated with 1 μm of ketoconazole after an 1.5 hours of treatment.

FIG. 47 shows the distribution of ketoconazole-treated cells as function of travel distance.

FIG. 48 shows the mean travel distances for the control (28.02 μm) and the ketoconazole-treated cells (20.97 μm).

FIG. 49 shows the dose response curve of U937 cells treated with varying concentrations of the drug topotecan. After 6 hours of exposure, escape velocity measurements were taken.

FIG. 50 shows the dose response curve for phorbol myristate acetate.

FIG. 51 illustrates the average measured escape velocities of U937 cells±5 hours of treatment with different concentrations of phorbol myristate acetate.

FIG. 52 illustrates the distribution of cells as measured by cell percentage as a function of measured escape velocity range for each of the PMA concentrations and the control of FIG. 51.

FIG. 53 illustrates the measured escape velocities of the U937 cells as a function of PMA concentration.

FIG. 54 illustrates the average escape velocity of U937 cells treated with 4 μg/ml camptothecin and 4 μg/ml topotecan. The control is also shown.

FIG. 55 is a table showing escape velocity measurements taken at 3, 6, 9, and 24 hour time periods for U937 cells incubated in media containing various concentrations of topotecan.

FIG. 56 illustrates the average measured escape velocities of the control and topotecan-treated cells at three, six, and nine hours and at 0.1, 1, and 10 μM topotecan.

FIG. 57 shows the measured escape velocities for the cells treated with 0.1, 1, and 10 μM topotecan after three hours of treatment.

FIG. 58 shows the measured escape velocities for the cells treated with 0.1, 1, and 10 μM topotecan after six hours of treatment.

FIG. 59 shows the measured escape velocities for the cells treated with 0.1, 1, and 10 μM topotecan after nine hours of treatment.

FIG. 60 shows the measured escape velocities for the cells treated with 0.1, 1, and 10 μM topotecan after twenty-four hours of treatment.

FIG. 61 illustrates the average escape velocities of the control sample as well as the topotecan-treated cells (0.1, 1, and 10 μM)± four hours of treatment.

FIG. 62 illustrates the average escape velocities of the control sample as well as the topotecan-treated cells,(0.1, 1, and 10 μM)± four hours of treatment.

FIG. 63 illustrates the distribution of U937 cells as a function of escape velocity± four hours of treatment with topotecan.

FIG. 64 illustrates the average escape velocity of the control cells as well as the topotecan-treated cells after six hours of treatment.

FIG. 65 illustrates the average escape velocity of the control cells as well as the topotecan-treated cells after six hours of treatment.

FIG. 66 shows the distribution of U937 cells as a function of escape velocity six hours after application of the topotecan.

FIG. 67 shows the mean escape velocities of the CCRF-CEM cell line and the CEM/C2 cell line after treatment with topotecan.

FIG. 68 shows the distribution of the CCRF-CEM cells and the CEM/C2 cells as a function of escape velocity range.

FIG. 69 illustrates the average escape velocity of camptothecin-treated cells (as well as the control) for differing concentrations of camptothecin (1.25 μM, 5 μM, 10 μM, and 20 μM).

FIG. 70 illustrates the average escape velocity of camptothecin-treated cells (as well as the control) for differing concentrations of camptothecin (1.25 μM, 5 μM, 10 μM, and 20 μM).

FIG. 71 illustrates the distribution of U937 cells as a function of escape velocity six hours after application of camptothecin.

FIG. 72 shows the distribution of control and transfected, receptor-producing cells over a range of escape velocities. This experiment tested the escape velocities of two CHO cell lines: one normal, one containing a vector causing an over-expression of a G-coupled protein kinase receptor, specifically, the CCK-1 receptor.

FIG. 73 shows the refractive index of a parental line of cells and three clone cell lines expressing varying levels of receptor protein over a period of three days.

FIG. 74 shows escape velocity measurements of three cell types, namely, B16.F10 wild type, B16.F10 sec 20, and B16.F10 sec 30.

FIG. 75A shows the time course escape velocity data of 293 ADGFP subpopulations through 24 hours of infection.

FIG. 75B shows the time course relative fluorescence of the 293 ADGFP subpopulation through the same 24 hours after infection.

FIG. 76 shows the escape velocity of Adeno-GFP cells that have been infected with varying amounts of virus. Measurements were taken 48 hours after infection. The cells were divided into three groups, dull, medium, and bright.

FIG. 77 shows a panel of images of infected (Adenovirus-GFP Transduction) and non-infected HeLa cells at 24 hours post-infection under fluorescence and standard lighting.

FIG. 78A shows an acquisition density plot showing three cell groups (dull, medium, and bright).

FIG. 78B shows the distribution of the infected cells in FIG. 78A.

FIG. 78C show a panel of images of the three cell groups (dull, medium, and bright) as well as the non-infected control group.

FIG. 79 graphically illustrates the result of an experiment on HeLa cells infected with recombinant adenovirus at 24 and 48 hours. Optophoretic shifts toward higher escape velocities can be seen at both 24 and 48 hours post-infection.

FIG. 80 shows the changes over time in escape velocity of wild type Staphylococcus aureus and an Erythromycin-resistant strain after exposure to Erythromycin.

FIG. 81 shows the results of another experiment in which 5 μg/ml of Erythromycin was applied to both the wild type Staphylococcus aureus and an Erythromycin-resistant strain. In this experiment, escape velocity measurements were made at time zero, 30 minutes post-treatment, and 1 hour post-treatment, and 2 hours post-treatment.

FIG. 82 graphically shows the escape velocity of the wild type and mutant strains of Saccharomyces cerevisiae.

FIG. 83 graphically illustrates the results of a fast scan analysis. The data show that fast scan analysis can be used to discriminate between the mutant and wild type strains of yeast.

FIG. 84 shows optophoretic differences of measured escape velocities for cells in different stages of the cell cycle.

FIG. 85 shows the distribution of escape velocities for live and heat-killed Staphylococcus aureus.

FIG. 86 shows the distribution of escape velocities for live and heat-killed Salmonella enterica.

FIG. 87 shows the distribution of escape velocities for live and heat-killed Saccharomyces cerevisiae.

FIG. 88 summarizes the results of experiments 1 and 2, showing the mean escape velocities for the live and heat-killed bacteria and yeast.

FIG. 89 shows the principles of operation of the fast scan method.

FIG. 90 illustrates a histogram of the travel distances for camptothecin-treated and untreated U937 cells at different time intervals.

FIG. 91 illustrates the mean travel distances of treated and untreated U937 cells at one hour, 2 hours, 3 hours, and 4 hours post-treatment with camptothecin.

FIG. 92 is a panel image of a FACS graph showing the cell number as a function of log annexin V binding for the control cells.

FIG. 93 is a panel image of a FACS graph showing the cell number as a function of log annexin V binding for the cells after one hour of exposure to camptothecin.

FIG. 94 is a panel image of a FACS graph showing the cell number as a function of log annexin V binding for the cells after two hours of exposure to camptothecin.

FIG. 95 is a panel image of a FACS graph showing the cell number as a function of log annexin V binding for the cells after three hours of exposure to camptothecin.

FIG. 96 is a panel image of a FACS graph showing the cell number as a function of log annexin V binding for the cells after four hours of exposure to camptothecin.

FIG. 97 illustrates the FACS annexin V profile in U937 cells that were not treated with camptothecin.

FIG. 98 illustrates the FACS annexin V profile in U937 cells that were treated with camptothexin after 1 hour of exposure.

FIG. 99 illustrates the FACS annexin V profile in U937 cells that were treated with camptothexin after 2 hours of exposure.

FIG. 100 illustrates the FACS annexin V profile in U937 cells that were treated with camptothexin after 3 hours of exposure.

FIG. 101 illustrates the FACS annexin V profile in U937 cells that were treated with camptothexin after 4 hours of exposure.

FIG. 102 is a graph of the relative fluorescent units (RFU) as a function of incubation time (hours) for the control cells and the camptothecin-treated cells.

FIG. 103 is a histogram of the travel distances of three cell types (MDA-435, HS578T, and HS578BST).

FIG. 104 is a graph of the mean travel distances for the three cell types (MDA-435, HS578T, and HS578BST).

FIG. 105 is a histogram of measured travel distances of a sample containing 100% non-cancerous HS578BST cells, a sample containing 10% (by number) of cancerous HS578T cells in mixture of both cancerous and non-cancerous HS578BST breast tissue cells, a sample containing 30% (by number) of cancerous HS578T cells in mixture of both cancerous and non-cancerous HS578BST breast tissue cells, a sample containing 60% (by number) of cancerous HS578T cells in mixture of both cancerous and non-cancerous HS578BST breast tissue cells, and a sample containing 100% cancerous HS578T cells.

FIG. 106 is a graph of the mean travel distances of the five samples discussed above with respect to FIG. 105.

FIG. 107 is a histogram of the measured travel distances of a sample containing 100% normal HS578BST cells, another having 50% (by number) of cancerous HS578T cells in a mixture of cancerous and non-cancerous cells, and a sample containing 100% cancerous HS578T cells.

FIG. 108 is a graph of the mean travel distances of the samples discussed above with respect to FIG. 107.

FIG. 109 is a histogram of the measured travel distances of a sample containing two very closely related cancer cells (MDA-MB-435 and MDA-MB-435S).

FIG. 110 is a graph of the mean travel distances of the samples discussed above with respect to FIG. 109.

FIG. 111 is a graph of the mean travel distances of various breast carcinoma cell lines (HS578T, MDA-ME-231, BT-20, MCF-7, MDA-ME-435, and MDA-MB-435S) as compared to non-cancerous HS578BST cells.

FIG. 112 is a histogram of the travel distances of six skin cell types: three of the cell types comprised normal skin cells (Detroit 551, CCD 1037, and Malme-3), the remaining three samples included the WM 266-4 malignant melanoma cell line, the matched WM 115 primary malignant melanoma cell line, and the 3-M malignant melanoma cell line. The 3-M malignant melanoma cells are matched with the Malme-3 (normal) cell line.

FIG. 113 is a graph of the mean travel distances of the skin cell types discussed above with respect to FIG. 112.

FIG. 114 illustrates the results (mean travel distance) of additional fast scan testing performed on various malignant melanoma cell lines (A375, RPMI 7950, SKMeI 5, WM 115, WM 266) as compared to non-cancerous Malme cells.

FIG. 115 illustrates the mean travel distances for chemically activated T-cells that were subject to Optophoretic analysis using a fast scan analysis. Three groups of T-cells were treated with various levels of phorbol mystirate acetate (PMA) and ionomycin to activate the T-cells.

FIG. 116 is a histogram of the travel distances of the chemically activated cells described in FIG. 115 above as well as the control.

FIG. 117 illustrates the FACS result for the control group of cells.

FIG. 118 illustrates the FACS result for cells treated with 5 ng/ml PMA and 500 ng/ml of ionomycin.

FIG. 119 illustrates the FACS result for cells treated with 0.5 ng/ml PMA and 50 ng/ml of ionomycin.

FIG. 120 illustrates the FACS result for cells treated with 0.05 ng/ml PMA and 5 ng/ml of ionomycin.

FIG. 121 shows the results of the BD ELISPOT confirmatory test for the control group of cells.

FIG. 122 shows the results of the BD ELISPOT confirmatory test for cells treated with 0.5 ng/ml PMA and 50 ng/ml of ionomycin.

FIG. 123 shows the results of the BD ELISPOT confirmatory test for cells treated with 5 ng/ml PMA and 500 ng/ml of ionomycin.

FIG. 124 shows the results of the BD ELISPOT confirmatory test for cells treated with 0.05 ng/ml PMA and 5 ng/ml of ionomycin.

FIG. 125 illustrates the mean travel distances for T-cells treated with different levels of PMA and ionomycin after 24 hours. The control group is also shown.

FIG. 126 is a histogram of the measured travel distances of the three groups of treated T-cells in addition to the control after 24 hours.

FIG. 127 illustrates the mean travel distances for T-cells treated with different levels of PMA and ionomycin after 48 hours. The control group is also shown.

FIG. 128 is a histogram of the measured travel distances of the three groups of treated T-cells in addition to the control after 48 hours.

FIG. 129 illustrates the FACS result for the control group of cells (untreated) after 24 hours.

FIG. 130 illustrates the FACS result for cells treated with 5 ng/ml PMA and 500 ng/ml of ionomycin after 24 hours.

FIG. 131 illustrates the FACS result for cells treated with 0.5 ng/ml PMA and 50 ng/ml of ionomycin after 24 hours.

FIG. 132 illustrates the FACS result for cells treated with 0.05 ng/ml PMA and 5 ng/ml of ionomycin after 24 hours.

FIG. 133 illustrates the FACS result for the control group of cells (untreated) after 48 hours.

FIG. 134 illustrates the FACS result for cells treated with 5 ng/ml PMA and 500 ng/ml of ionomycin after 48 hours.

FIG. 135 illustrates the FACS result for cells treated with 0.5 ng/ml PMA and 50 ng/ml of ionomycin after 48 hours.

FIG. 136 illustrates the FACS result for cells treated with 0.05 ng/ml PMA and 5 ng/ml of ionomycin after 48 hours.

FIG. 137 illustrates the mean travel distances for untreated T-cells (control) as well as T-cells that were incubated with anti-CD3 antibody. Measurements were made after 24 hours of incubation.

FIG. 138 is a histogram of the travel distances of the cells described above with respect to FIG. 137 after 24 hours of incubation.

FIG. 139 illustrates the mean travel distances for untreated T-cells (control) as well as T-cells that were incubated with anti-CD3 antibody. Measurements were made after 48 hours of incubation.

FIG. 140 is a histogram of the travel distances of the cells described above with respect to FIG. 139 after 48 hours of incubation.

FIG. 141 shows the FACS analysis results of T-cells that were not treated with anti-CD3 antibody after 24 hours.

FIG. 142 shows the FACS analysis results of T-cells that were treated with anti-CD3 antibody after 24 hours.

FIG. 143 shows the FACS analysis results of T-cells that were not treated with anti-CD3 antibody after 48 hours.

FIG. 144 shows the FACS analysis results of T-cells that were treated with anti-CD3 antibody after 48 hours.

FIG. 145 is a histogram of the travel distance of PMA-treated HL-60 cells at 16 hours, 24 hours, 40 hours, and 72 hours post-treatment. The control is also shown.

FIG. 146 shows the mean travel distances of PMA-treated HL-60 cells at 16 hours, 24 hours, 40 hours, and 72 hours post-treatment. The control is also shown.

FIG. 147 illustrates the FACS CD11b expression profile of PMA-treated HL-60 cells as well as the control.

FIG. 148 illustrates a histogram of the travel distance of DMSO-treated HL-60 cells at 16 hours, 24 hours, 40 hours, and 72 hours post-treatment. The control is also shown.

FIG. 149 shows the mean travel distances of DMSO-treated HL-60 cells at 16 hours, 24 hours, 40 hours, and 72 hours post-treatment. The control is also shown.

FIG. 150 illustrates the FACS CD11b expression profile of DMSO-treated HL-60 cells as well as the control.

FIG. 151(a) illustrates uninduced pre-adipocytes stained with oil red.

FIG. 151(b) illustrates eight day induced adipocytes stained with oil red.

FIG. 152(a) illustrates uninduced pre-adipocytes stained with BODIPY 505/515 fluorophore.

FIG. 152(b) illustrates eight day induced adipocytes stained with BODIPY 505/515 fluorophore.

FIG. 153 illustrates a histogram of the displacement of the 3T3-L1 cells at day 2, day 4, day 6, and day 8 post-induction. The uninduced control cells are also shown.

FIG. 154 illustrates the mean travel distances of the 3T3-L1 cells at day 2, day 4, day 6, and day 8 post-induction. The uninduced control cells are also shown.

FIG. 155 illustrates the relative Optophoretic shift in mean travel distance over the eight day post-induction period for the 3T3-L1 cells.

FIG. 156 is a graph of the fluorescent level of 3T3-L1 cells stained with BODIPY 505/515 over the eight day post-induction period.

FIG. 157 is a graph of the relative signal from the BODIPY 505/515 assay as compared to Optophoretic analysis.

FIG. 158 illustrates normalized levels of PPARγ and C/EBPα mRNA over the eight day post-induction period.

FIG. 159 is a graph of normalized levels of mRNA coding for the protein Leptin at 2, 4, 6, and 8 days post-induction.

FIG. 160 is a graph of normalized levels of mRNA coding for aP2 at 2, 4, 6, and 8 days post-induction.

FIG. 161 is a histogram of the displacement of 3T3-L1 cells at days 2, 3, 4, and 5 post-induction.

FIG. 162 illustrates the mean travel distances of the uninduced control 3T3-L1 cells as well as the induced 3T3-L1 cells at days 2, 3, 4, and 5 post-induction.

FIG. 163 is a graph of the fluorescent level of 3T3-L1 cells stained with BODIPY 505/515 over the five day post-induction period.

FIG. 164 illustrates normalized levels of PPARγ and C/EBPα mRNA over the five day post-induction period.

FIG. 165 is a graph of normalized levels of mRNA coding for aP2 at 2, 3, 4, and 5 days post-induction.

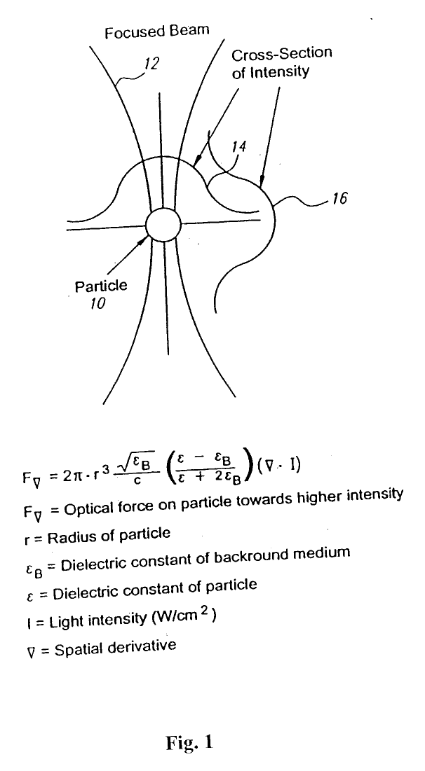

### DETAILED DESCRIPTION OF THE PREFERRED EMBODIMENTS

Definitions

The following definitions are provided for an understanding of the invention disclosed herein.

“Biological Property” means a distinct phenotype, state, condition, or response of a cell or group of cells, for example, whether a cell has been infected by a virus, the degree to which a cell expresses a particular protein, the stage in the cell cycle a particular cell is presently at, whether the cell is affected by the presence of a chemical compound, a particular phenotype of the cell, whether a ligand is bound to the surface of a cell, cytoskeletal changes in the cell, whether a cell is decorated with antibodies, the presence or absence of a cellular component (e.g., an organelle or inclusion body), a change in one or more cellular components, the toxicity of chemical compounds, a physical property of a cell or population of cells, a response of a cell or population of cells to an external stimulus, cellular motility, membrane fluidity, state of differentiation, viability, size, osmolarity, adhesion, secretion, cell/cell interactions, activation, and cell growth.

“Determining” is meant to indicate that a particular phenotype, state, condition, or response is ascertained.

“Dielectric constant” is defined to be that property which determines the electrostatic energy stored per unit volume for unit potential gradient. (See, e.g., the New IEEE Standard Dictionary Of Electrical And Electronics Terms, ©1993).

The “escape velocity” is defined as the minimum speed at which an interrogated cell or particle no longer tracks the moving optical gradient.

The “optical dielectric constant” is the dielectric constant of a particle or thing at optical wavelengths. Generally, the optical wavelength range is from 150 Å to 30,000 Å.

An “optical gradient field” is an optical pattern having a variation in one or more parameters including intensity, wavelength or frequency, phase, polarization or other parameters relating to the optical energy. When generated by an interferometer, an optical gradient field or pattern may also be called an optical fringe field or fringe pattern, or variants thereof.

A “moving optical gradient field” is an optical gradient field that moves in space and/or time relative to other components of the system, e.g., particles or objects to be identified, characterized, selected and/or sorted, the medium, typically a fluidic medium, in contact with the particles, and/or any containment or support structure.

An “optical scattering force” is that force applied to a particle or thing caused by a momentum transfer from photons to material irradiated with optical energy.

An “optical gradient force” is one which causes a particle or object to be subject to a force based upon a difference in dielectric constant between the particle and the medium in which it is located.

“Optophoresis” or “Optophoretic” generally relates to the use of photonic or light energy to obtain information about or spatially move or otherwise usefully interact with a particle.

“Optophoretic constant” or “optophoretic signature” or “optophoretic fingerprint” refer to the parameter or parameters which distinguish or characterize particles for optical selection, identification, characterization or sorting.

“Separation” of two objects is the relative spatial distancing over time of a particle from some other reference point or thing.

“Sorting” involves the separation of two or more particles in a meaningful way.

### DESCRIPTION OF EXEMPLARY APPARATUS

Optical Components—Generation of Moving Optical Gradient Field.

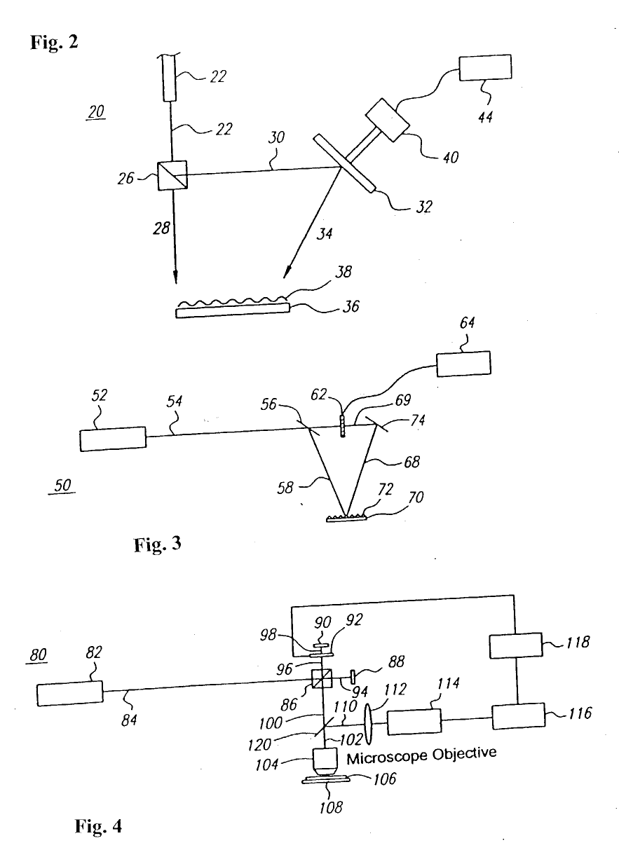

FIGS. 2-10 describe various systems for generation of optical patterns, sometimes termed fringe patterns or optical fringe patterns, including, but not limited to, a moving optical gradient field pattern. These exemplary embodiments are intended to be illustrative, and not limiting, as other apparatus may be utilized to generate the optical fields and forces to achieve the desirable results of these inventions.

The points raised in discussions of specific embodiments may be considered to be generally applicable to descriptions of the other embodiments, even if not expressly stated to be applicable.

The light source for use with systems has certain generally desirable properties. As to wavelength, the wavelength will generally be chosen based upon one or more considerations. In certain applications, it may be desirable to avoid damage to biological materials, such as cells. By choosing wavelengths in ranges where the absorption by cellular components, mostly water, are minimized, the deleterious effects of heating may be minimized. Wavelengths in the range from approximately 0.3 μm to approximately 1.8 μm, and more preferably, from substantially 0.8 to substantially 1.8 μm, aid in reducing biological damage. However, even for biological applications, a laser having a wavelength generally considered to be damaging to biological materials may be used, such as where the illumination is for a short period of time where deleterious absorption of energy does not occur. In yet other applications, it may be desirable to choose a wavelength based upon a property of the particle or object under consideration. For example, it may be desirable to choose the wavelength to be at or near an absorption band in order to increase (or decrease) the force applied against a particle having a particular attribute. Yet another consideration for wavelength choice may be compatibility with existing technology, or a wavelength naturally generated by a source. One example would be the choice of the wavelength at 1.55 μm. Numerous devices in the 1.55 μm wavelength region exist commercially and are used extensively for telecommunications applications.

Generally, the light sources will be coherent light sources. Most typically, the coherent light source will consist of a laser. However, non-coherent sources may be utilized, provided the system can generate the forces required to achieve the desired results. Various laser modes may be utilized, such as the Laguerre-Gaussian mode of the laser. Furthermore, if there is more than one light source in the system, these sources can be coherent or incoherent with respect to each other.

The spot size or periodicity of the intensity pattern is preferably chosen to optimize the effective results of the illumination. In many applications, it is desirable to have a substantially uniform gradient over the particle, e.g., cell, to be interrogated such that the dielectric properties of the entire particle (cell) contribute to the resulting force. Broadly, the range varies from substantially 1 to substantially 8 times the size (diameter or average size) of the particle or object, more preferably, the range is from substantially 2 to substantially 4 times the size. Various methods and systems known to those skilled in the art may be utilized to achieve the desired spot size or periodicity, e.g., using a defocused beam or a collimated beam having the desired size. The typical characterization of the radius of the spot is the 1/e² radius of the beam intensity. For many applications, including cellular applications, the beam size will be on the order of 10 microns, though sometimes as small as five microns, and in even certain other occasions, as small as two microns. In certain applications, it is desirable to have the periodicity of the illumination in the range from substantially 1 to substantially 2 times the size (diameter or average size) of the particle or object. For many biological applications, a periodicity of from substantially 5 μm to 25 μm, and more preferably from 10 μm to 20 μm. Certain applications may utilize smaller sizes, e.g., for bacteria, or larger sizes, e.g., for larger particles. In yet other applications, it may be desired to utilize a spot size smaller than the particle or object, such as where interrogation of a sub-cellular region is desired.

The examples of systems for generating intensity patterns, described below, as well as other systems for generating intensity patterns useful for the subject inventions include various optical components, as well as a control system to generate the desired pattern, intensity profile or other gradient, such as a moving optical field gradient. Various optical systems may be adapted for use in the systems of the invention, so as to effectively carry out the methods and achieve the results described herein. Exemplary systems which may be adapted in whole or in part include: Young's slits, Michelson interferometer, Mach-Zender interferometer, Haidinger circular fringe systems, Fresnel mirror interferometer, plane-parallel plate interferometer, Fabry-Perot interferometer and any other system for generating an optical gradient intensity pattern or fringe pattern.

Turning now to a detailed description of exemplary systems for use with the subject inventions. FIG. 2 shows an optical component description of a system 20 generally configured to generate a moving optical gradient field pattern to provide a force on one or more particles provided to the system 20. The optical forces may then be used for characterization, identification, selection and/or sorting of the particles. A light source 22, preferably a laser, generates a first beam 24 directed toward beam splitter 26. Beam splitter 26 may be of any mode or type known to the art, such as a prism beam splitter, consistent with the goals and objects of this invention. A first transmitted beam 28 passes through the beam splitter 26. A first reflected beam 30 reflects from the beam splitter 26 to a reflective surface 32, typically a mirror, to generate a second reflected beam 34. The first transmitted beam 28 and second reflected beam 34 interfere and generate an intensity pattern 38, generally being located at the operative portion of the slide or support 36 where the light would interact with the particle or object of interest. The optical pattern 38 moves relative to other objects, e.g., the particles, the substrate, and/or the fluidic medium containing the particles, by virtue of a change in the optical path length between the first transmitted beam 28 and the combination of the first reflected beam 30 and second reflected beam 34. Mirror 32 is movable, by actuator 40. One example of an actuator 40 could comprise a motor and screw system to move mirror 32. Numerous alternative'structures for moving mirror 32 are known to the art, e.g., piezoelectric systems, oscillating mirror systems and the like.

FIG. 3 shows a two-beam interference based system. A source of coherent light, such as laser 52, generates a first beam 54 directed to a beam splitter 56. A first reflected beam 58 is directed toward the sample plate 70 and a first transmitted beam 60 is directed to a modulator, such as a phase modulator 62. The phase modulator 62 may be of any type known to those skilled in the art. Phase modulator 62 is under control of the control system 64 and results in modulated beam output 66 which is directed to a mirror 74. The modulated beam 66 reflects from mirror 74 to generate the second reflected beam 68 which is directed to the sample plate 70. The first reflected beam 54 and second reflected beam 68 generate a pattern 72 at the operative interface with the sample plate 70. The control system 64 is connected to the phase modulator 62 so as to cause the pattern 72 to move relative to the objects within the system 50, such as the sample plate 70.

FIG. 4 shows an optical component diagram of an interferometer system 80. A light source, such as laser 82, generates a first light beam 84 directed to beam splitter 86. An interferometer composed of the first mirror 88 and second mirror 90 generate an output beam 100 having the desired beam properties, including the desired gradient properties. The first beam 84 passes through beam splitter 86 to generate a first transmitted beam 94 directed to first mirror 88. The reflected beam retraces path 94 to the beam splitter 86. The first reflected beam 96 passes through phase modulator 92 to generate first modulated beam 98 directed to the second mirror 90. The reflected beam from second mirror 90 retraces the path 98 through the phase modulator 92 and beam 96 to the beam splitter 86. The beam 100 is output from the interferometer section of the system 80 and directed toward the microscope objective 104.

The objective 104 is directed toward the sample plate 106. Optionally, a mirror 108, most preferably a planar mirror, may be disposed beneath the sample plate 106. The mirror 108 is oriented so as to provide reflected light onto the sample plate 106 bearing or containing the particles or objects under analysis or action of the system 80. The scattering force caused by the beam 102 as initially illuminates the sample plate 106 may be counteracted, in whole or in part, by directing the reflected radiation from mirror 108 back toward the sample. As discussed more in the section relating to surface effects, below, the reflected light and the upward scattering force reduce the overall effects of the scattering forces, such that the gradient forces may be more effectively utilized.

FIG. 4 includes an optional imaging system. The light 102 from the objective 104 is reflected by the beam splitter 120 generating third reflected beam 110 which is directed toward imaging optics 112. The optics 112 image the light on a detector 114, such as a charge couple device (CCD) detector. The output of the detector 114 may be provided to an imaging system 116. The imaging system 116 may optionally include a display, such as a monitor (CRT, flat panel display, plasma display, liquid crystal display, or other displays known to those skilled in the art). The imaging system 116 may optionally include image enhancement software and image analysis software, recording capability (to tape, to optical memory, or to any other form of memory known to those skilled in the art).

A control system 118 controls the modulator 92 so as to generate the desired optical force pattern within the system 80. Optionally, the imaging system 116 may be coupled to the control system 118. A feedback system may be created whereby the action of the particles on the sample plate 106 may be imaged through the system 116 and then utilized in the control system analysis to control the operation of the overall system 80.

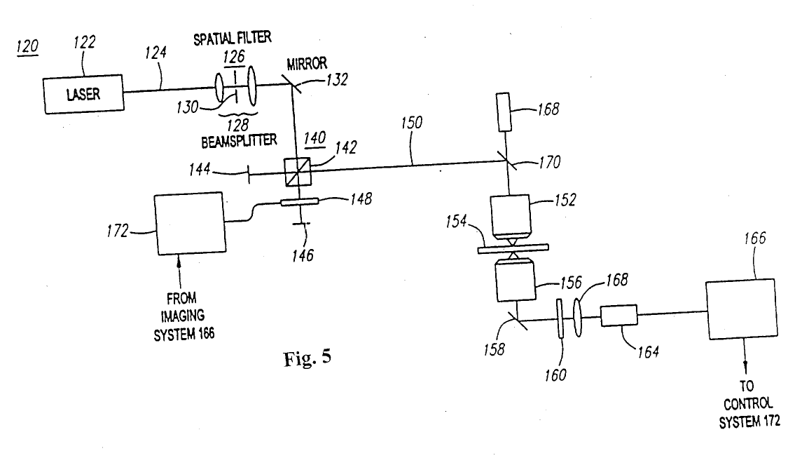

FIG. 5 shows a interferometer based system 120. A light source, such as laser 122, generates a first beam 124 directed toward an optional spatial filter 126. The spatial filter 126 would typically include lenses 128 and a spatial filter aperture 130. The aperture typically is round. The spatial filters serves to collimate the laser beam and to produce a smooth intensity profile across the wavefront of the laser beam. The interferometer 140 includes first mirror 146 and second mirror 144, as well a beam splitter 142. The phase modulator 148 is disposed within one of the two arms of the interferometer 140.

As shown in FIG. 5, a mirror 132 is optionally disposed to reflect the light from the source 122 to the interferometer 140. As will be appreciated by those skilled in the art, optical systems may include any number or manner of components designed to transfer or direct light throughout the system. One such example is the planar mirror 132 which merely serves to direct the radiation from one major component, e.g., the spatial filter, to another major component, e.g., the interferometer 140. In addition to mirrors, other common transfer components may include fiber optics, lenses, beam splitters, diffusers, prisms, filters, and shaped mirrors.

Beam 150 exits the interferometer 140 and is directed toward objective 152 and imaged at or near the sample plate 154. As shown, a dichroic mirror 170 serves to reflect the light 150, but to also permit passage of light from source 168, such as a fiber providing radiation from a source through the dichroic mirror 170 and objective 152 to illuminate the operative regions of the sample plate 154.

Optionally, a detection system may be disposed to image the operative portions of the sample plate 154. As shown, objective 156 is disposed beneath the sample plate 154, with the output radiation being transferred via mirror 158 to an imaging apparatus 164, such as a charge couple device (CCD). Optionally, an infrared filter 160 may be disposed within the optical path in order to select the desired wavelengths for detection. The output of the detector 164 is provided to an imaging system 166. As described in connection with other figures, the imaging system 166 may include image enhancement and image analysis software and provide various modes of display to be user. Optionally, the imaging system 166 is coupled to the control system 172 such as when used for feedback.

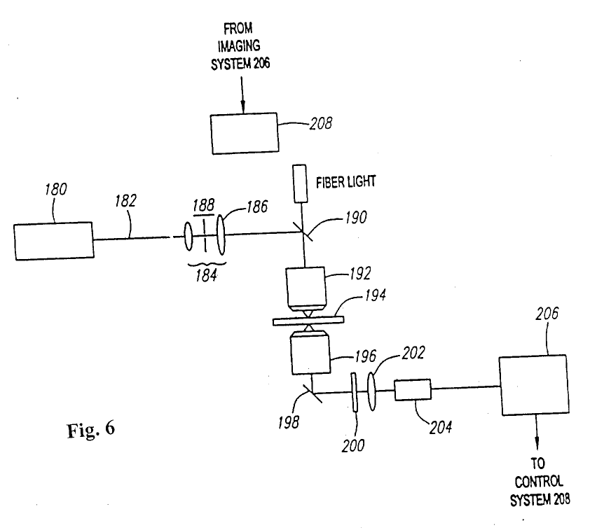

FIG. 6 shows an optical system having illumination of a sample plate 194 from the top side and imaging from the bottom side. A laser 180 generates a first beam 182 which optionally passes through a spatial filter 184. The spatial filter as shown includes lens 184 and aperture 188. The output of the spatial filter 184 passes through the objective 192 and is imaged onto the sample plate 194. The sample plate 194 and material supported on it may be imaged via an objective 196. An optional mirror 198 directs radiation to an optional filter 200 through an imaging lens 202 onto the detector 204. The detector 204 is coupled to an imaging system 206. Preferably, the imaging system 206 provides information to a control system 208 which controls various optical components of the system.

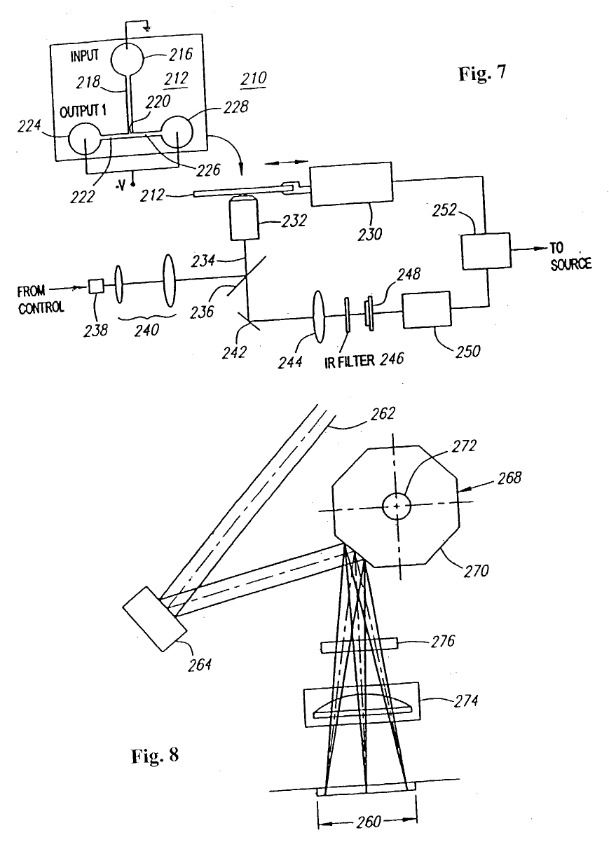

FIG. 7 shows an optical system interfacing a sample plate which includes bounded structures. The system 210 includes a sample plate 212 which optionally includes microfluidic channels. Alternatively, the sample plate 212 may support a separate structure containing the microfluidic channels. As one exemplary structure formed from the microfluidic channels, a “T” sorting arrangement is shown for a simple, though useful, example. An input reservoir 216 connects to a first channel 218 which terminates in a T at intersection 220. A first output channel 222 couples to a first output reservoir 224. A second output channel 226 couples to a second output chamber 228. As shown, the input chamber is coupled to ground and the first output chamber 224 and second output chamber 228 are connected to −V. The fluidic channel structures are discussed in more detail, below.

The microscope objective 232 serves to both provide the optical radiation to the sample plate 222 as well as to provide the imaging of the system. A light source 238, such as a laser, or more particularly, a laser diode, generates light which may be imaged by optics 240. A dichroic beam splitter 236 directs the radiation to the microscope objective 232. As shown, the objective has a magnification power of 100. For the biological applications, a magnification range of from 1 to 200 is desired, and more preferably, from 10 to 100. The objective 232 has a 1.25 numerical aperture. The preferable range of numerical apertures for the lenses is from 0.1 to 1.50, and more preferably from 0.4 to 1.25. The output from the objective 232 passes through the beam splitter 236, reflects from optional mirror 242 through optics (e.g., lens) 244, through the optional filter 246 to the imaging device 280. The imaging device, shown as a CCD, is connected to the imaging system 282. The output of the imaging system 282 is optionally coupled to the control system 284. As shown, the control system 284 controls both the translation stage 232 connected to the sample plate 212, as well as to the light source 238.

FIG. 8 shows a system for generating an intensity pattern within the scanned area 260. An input beam 262, such as from a coherent light source, such as a laser, is directed toward the system. A first oscillating component 264, such as a galvanometer or resonant scanner, intercepts the input beam 262 and provides a first degree of motion to the beam. The beam is directed to a polygonal mirror 268 which contains multiple faces 270. As the polygonal mirror 268 rotates around axis 272, the light is swept across the scanner area 260. Lens 274 are provided as required to appropriately image the light into the scanned area 260. Optionally, a mask or other pattern 276 may be disposed within the optical pathway so as to provide for the variation of the optical forces within the scanned area 260. Any of a wide variety of techniques for generating either the oscillatory motion or the scanning via the polygonal mirror are known to those skilled in the art.

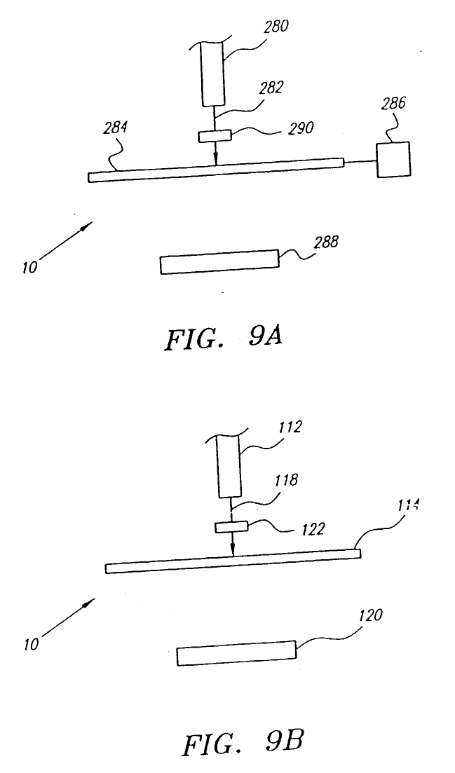

FIG. 9 shows a system utilizing masks to generate an optical force pattern. A source 280, such as a laser, generates a beam 282 directed to toward a mask 284. Optionally, a phase modulator 290 may be disposed between the source 280 and the mask 284. Optionally, the mask 284 may be moved, such as by actuator 286, which may be a motor, piezoelectric driven system, microelectromechanical (MEMs), or other driving structures known to those skilled in the art. The optical mask 284 creates a desired light intensity pattern adjacent the sample plate 288. The optical mask 284 may modulate any or all of the components of the light passing there through, include, but not limited to, intensity, phase and polarization. The mask 284 may be a holographic mask which, if used, may not necessarily require coherent light. Other forms of masks, such as spatial light modulators may be utilized to generate variations in optical parameters.

Yet another mirror arrangement consists of utilizing a micromirror arrangement. One such micromirror structure consists of an array of mirrors, such as utilized in the Texas Instrument Digital Micromirror product.

FIG. 10 shows an alternate system for illumination in which multiple sources 290 are directed toward the sample plate or surface 294. Each source 290 is controlled by control system 296, with the various outputs 292 from the sources 290 illuminating the surface of the support 294.

The imaging system may serve function beyond the mirror imaging of the system. In addition to monitoring the intensity, size and shape of the optical fringes, it may be used for purposes such as calibration.

Optical Forces

The apparatus and methods of the instant inventions utilize, at least in part, forces on particles caused by light. In certain embodiments, a light pattern is moved relative to another physical structure, the particle or object, the medium containing the particle or object and/or the structure supporting the particle or object and the medium. Often times, a moving optical pattern, such as moving optical gradient field moves relative to the particles. By moving the light relative to particles, typically through a medium having some degree of viscosity, particles are separated or otherwise characterized based at least in part upon the optical force asserted against the particle. While most of the description describes the light moving relative to other structures, it will be appreciated that the relative motion may be achieved otherwise, such as by holding the light pattern stationary and moving the subject particle, medium and/or support structure relative to the optical pattern.

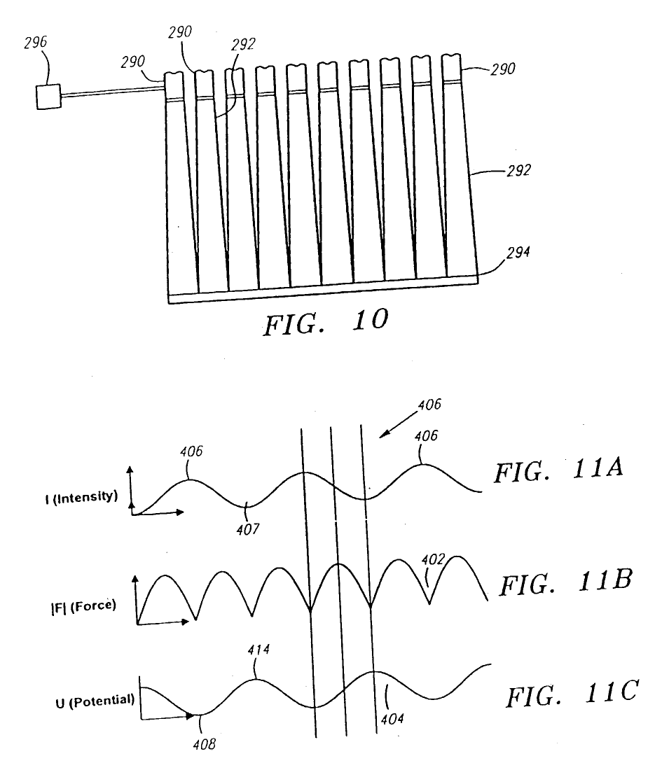

FIGS. 11A, 11B and 11C depict, respectively, the optical intensity profile, the corresponding optical force on a particle or cell and the corresponding potential energy of the particle in the optical intensity profile as a function of distance (x). FIG. 11A shows the intensity profile generated and applied against one or more particles. As shown, the intensity varies in a undulating or oscillating manner. The intensity, as shown, shows a uniform periodicity and symmetric waves. However, the intensity variations may be symmetric or asymmetric, or of any desired shape. The period may be fixed or may be variable. FIG. 11B shows the absolute value of the force as a function of position. The force is the spatial derivative of the intensity. FIG. 11C shows the potential energy as a function of position. The potential energy is the integrated force through a distance.

The profiles of FIGS. 11A-11C are shown to be generally sinusoidal. Generally, such a pattern would result from interference fringes. Differing profiles (of intensity, force and potential energy) may be desired. For example, it may be desirable to have a system where the potential energy well is relatively flat at the bottom and has steeper sides, or is asymmetric in its form.

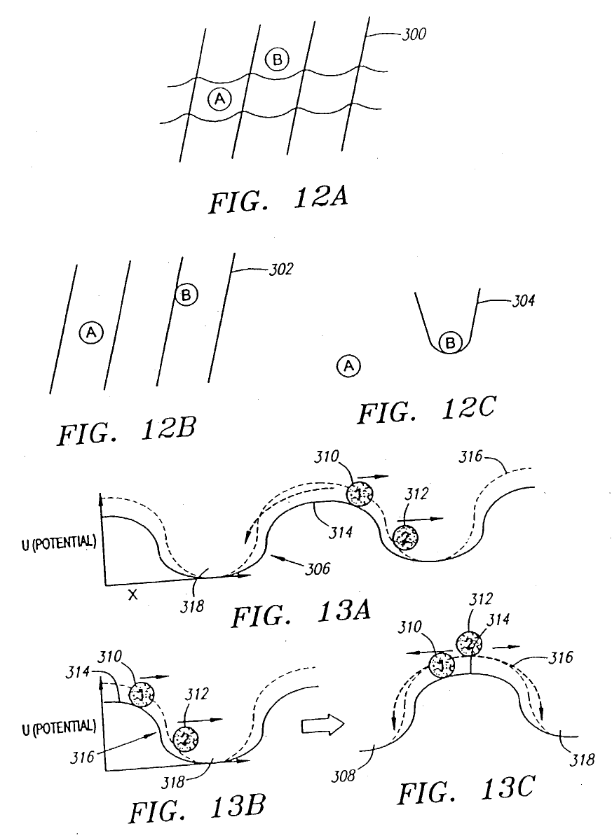

FIGS. 12A and 12B show two particles, labeled “A” and “B”. in FIG. 12A, the particles are shown being illuminated by a two-dimensional intensity pattern 300. FIG. 12B shows the position of particles A and B at a later moment of time, after the intensity pattern has moved to position 302. In this example, the optical force has caused particle B to move relative to its prior position. Since the effect of the optical pattern 300 on particle A was less than on particle B, the relative positions of particles A and B are different in FIG. 12B as compared to FIG. 12A.

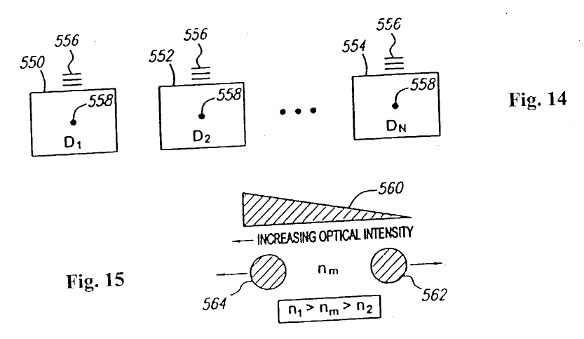

In one implementation of the system, the position of particles A and B in FIG. 12A would be determined. The system would then be illuminated with the desired gradient field, preferably a moving optical gradient field, and the system then imaged at a later point in time, such as shown in FIG. 12B. The absence of motion, or the presence of motion (amount of motion, direction of motion, speed of motion, etc.) may be utilized to characterize, or analyze the particle or particles. In certain applications, it may be sufficient to determine the response of a single particle to a particular optical pattern. Thus, information may be derived about the particle merely from the fact that the particle moved, or moved in a particular way or by a particular amount. That information may be obtained irrespective of the presence or absence of other particles. In yet other applications, it is desirable to separate two or more particles. In that case, by comparing the position of the particles relative to each other such as in FIG. 12A versus 12B, information regarding the particle may be obtained. Having determined which particle is the desired particle, assume for purposes of discussion to be particle B. the particle may then be separated from the other particles. As shown in FIG. 12C, an optical tweezer intensity profile 304 may be used to capture and remove particle B. Alternatively, as will be discussed in connection with FIGS. 14-19, the selected particle may be removed by other means, such as by fluidic means.

By utilizing a property of the particle, such as the optical dielectric constant, the light forces serve to identify, select; characterize and/or sort particles having differences in those attributes. Exposure of one or more particles to the optical force may provide information regarding the status of that particle. No separation of that particle from any other particle or structure may be required. In yet other applications, the application of the optical force causes a separation of particles based upon characteristics, such that the separation between the particles may result in yet further separation. The modes of further separation may be of any various forms, such as fluidic separation, mechanical separation, such as through the use of mechanical devices or other capture structures, or optically, such as through the use of an optical tweezer as shown in FIG. 12C, by application of a moving optical gradient, or by any other mode of removing or separating the particle, e.g., electromagnetic, fluidic or mechanical.

FIGS. 13A, 13B and 13C show potential energy as a function of distance for one exemplary mode of operation. The figures show particle 1 and particle 2 displaced in the x dimension relative to one another. The physical positioning of the two particles would typically be in the same plane, e.g., the same vertical plane. The figures show the potential energy of the particle. In FIG. 13A, particle 1 310 is subject to light intensity pattern creating the potential energy profile 314. Particle 2 312 is subject to the same light intensity pattern but is subject to the second potential energy profile 316. The second potential energy profile 316 is different from the first potential energy profile 314 because the dielectric constants are different between particle 1 310 and particle 2 312. In FIG. 5A, the light intensity pattern is moving toward the right. As the potential energy profiles 314, 316 move to the right, the particles 310, 312 experience different forces. Particle 1 310 will experience a smaller force as compared to particle 2 312, as depicted by the size of the arrows adjacent the particles. The force experienced by the particles is proportional to the spatial derivative of the potential energy. Thus, particle 2 312 being on a relatively “steeper” portion of the potential energy “wave” would be subject to a larger force. In FIG. 5A, the translation speed of the potential energy waves may be set to be larger than the speed at which particle 1 310 may move forward through the medium in which it is located. In that event, particle 1 310 may be subject to a force toward the left, FIG. 13A showing an arrow depicting the possible backward or retrograde motion of particle 1 310. The potential energy wells have a minimum 318 into which the particles would settle, absent motion or translation of the potential energy patterns 314, 316.

FIG. 13B shows particle 1 310 and particle 2 312 subject to the first potential energy 314 and second potential energy 316, respectively. As the potential energy patterns 314, 316 translate to the right, the particles 310, 312 are subject to a force to the right, though in different amounts as depicted by the relative size of the arrows. FIG. 13C shows the potential energy profiles 314, 316 after the potential energy profiles of FIG. 13B have been moved so as to place the potential energy maximum between particle 1 310 and particle 2 312. By “jerking” the intensity profiles 314, 316 forward quickly, particle 1 310 is then located on the “backside” of the potential energy “wave”, and would be subject to a force to the left. The path of motion is then shown by the dashed arrow from particle 1 310. In contrast, particle 2 312 remains on the “front side” of the potential energy wave 316 and is subject to a force to the right. The effect of this arrangement is to cause further physical separation between particle 1 310 and particle 2 314. The potential energy profiles 314, 316 must be moved forward quickly enough such that the potential energy maximum is located between the particles to be separated, as well as to insure that the particle on the “backside” of the potential energy wave is caused to move away from the particle on the “front side” of the wave.

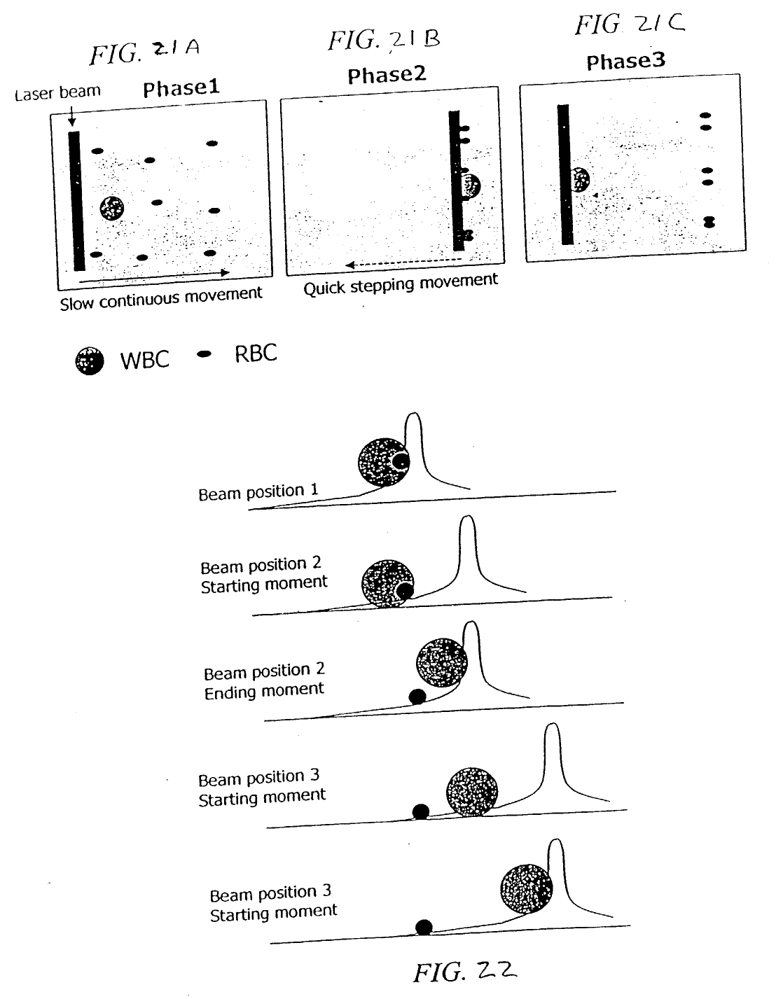

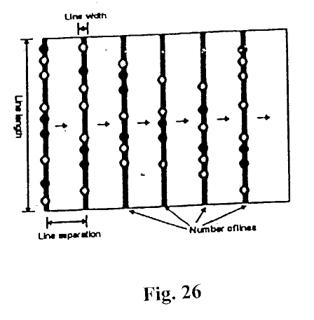

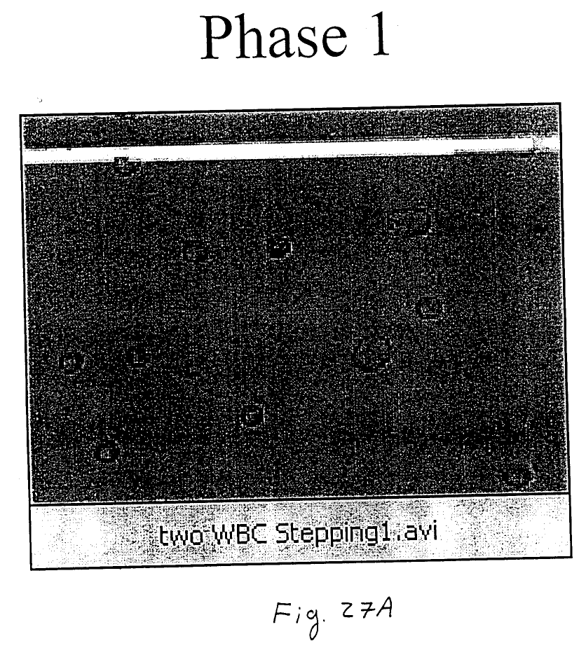

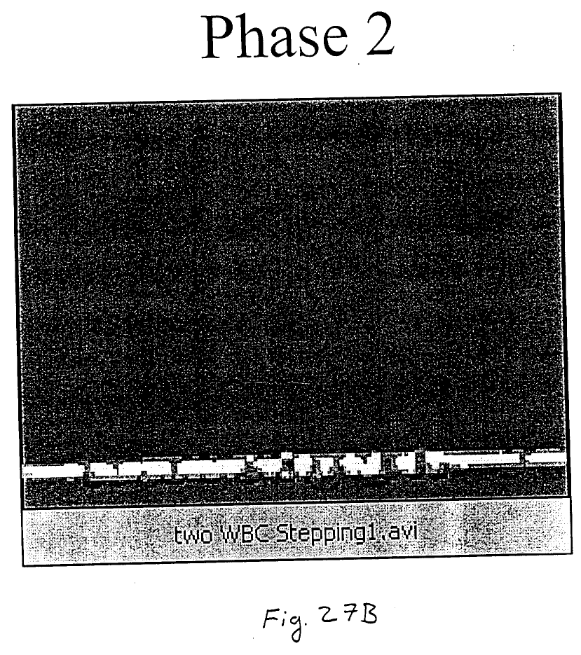

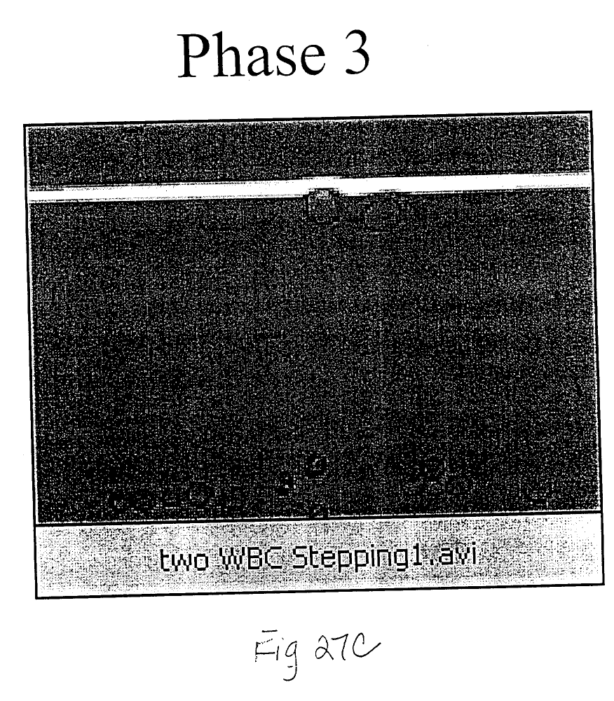

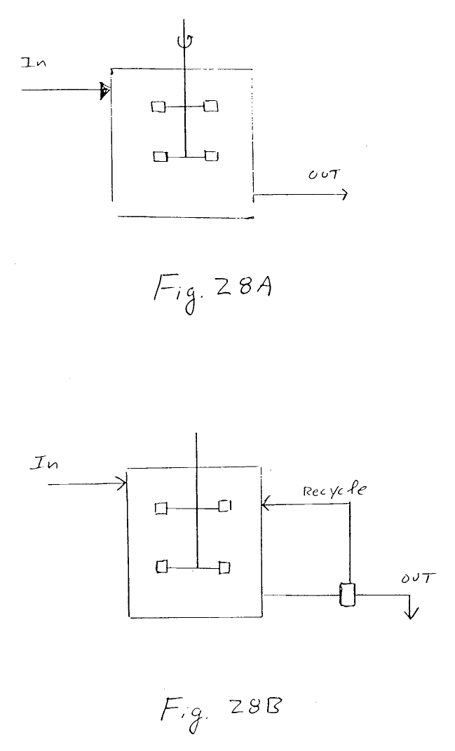

FIGS. 21A, B and C show a time series depiction of a technique for the identification, characterization and/or sorting of particles. In FIG. 26A, a population of particles is subject to a beam of light, preferably a line of light shown as the laser beam in FIG. 26A. The direction of illumination is into the plane of the population of particles. The line of light is moved relative to the particle population to physically organize the particle population. Optionally, the beam is moved at a speed which is sufficiently slow as to permit capture of all desired particles and to move the particles to the desired location within the system. FIG. 26B shows phase two in which the line of light is moved relative to the now physically arranged line of particles. Optionally, the relative direction of the light relative to the particles in phase one is in one direction, and in phase two, in an opposite direction. In phase two, the line of light is moved relative to the particles in a relatively quick, stepping movement. The speed of movement is at least great enough to effect the desired separation of particles. Those particles which are subject to a greater force are selectively moved from the physical position of the arranged particles in phase two. FIG. 26C shows the illumination of the white blood cell particle (shown as the larger particle in the shading) being effectively separated from the red blood cells (shown as the relatively smaller dark ellipses).

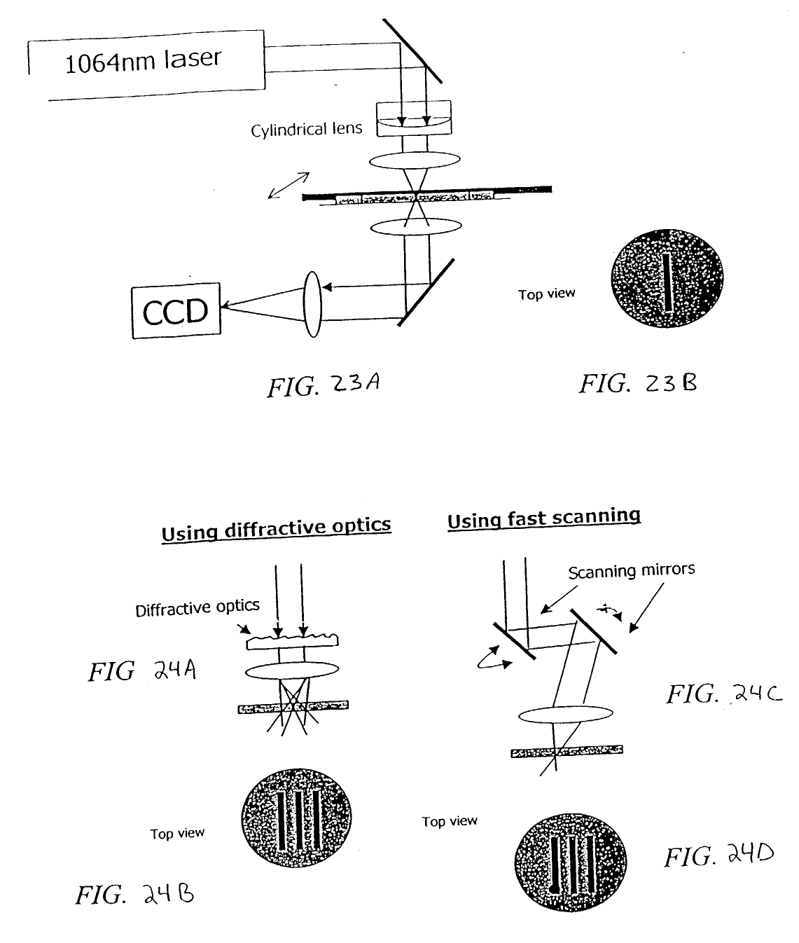

FIG. 22 is a time series graph of the intensity and its position relative to the population of particles. Beam position 1 shows the intensity profile within a few seconds after the beam is turned on. It has sometimes been observed that the particles are slightly offset from the intensity maximum. Beam position 2 depicts the stepping movement referred to in phase two (FIG. 26B). As can be seen, the white blood cell is subjected to a larger gradient force with the result being that it is physically moved more at the ending moment of beam position 2 than is the red blood cell. Beam position 3 depicts yet a subsequent step movement where again the white blood cell is subject to a larger gradient force resulting in its movement to the right. As the beam position continues to move to the right, the distance between the intensity peak and the particles remaining behind, e.g., the red blood cells, grows greater, and accordingly, the gradient force felt by the particles diminishes.

FIG. 23A shows a cross-sectional arrangement for generating a single line for use in this technique. A laser is directed through a cylindrical lens toward the system. Focusing optics maybe utilized as are described elsewhere herein, and are well known to those skilled in the art. An imaging system, such as the CCD imaging system depicted captures the information from the system. The light pattern may be moved relative to the particles, or alternately, the particles may be moved relative to the light by translating the stage. Preferably, the line of illumination has a relatively uniform intensity, which may be achieved, for example, by modifying the curvature of the lens.

FIGS. 24A and 24B show a cross-section of a alternate arrangement to generate one or more lines of light. Diffractive optics receive an incident beam, which when focused through the optics generate one or more lines of light within the sample region. FIGS. 24C and 24D show yet another alternate arrangement for generating one or more lines. A scanning mirror system, such as those utilizing two scanning mirrors generally oscillating around an access running through the plane of the mirror, where the axis are non-colinear, they result in a generation of one or more lines. Generally, one of the mirrors moves at a substantially higher rate than the other mirror. Alternates to the multiple scanning mirror system may be utilized, such as an acoustic/optic device for generating the desired intensity patterns.

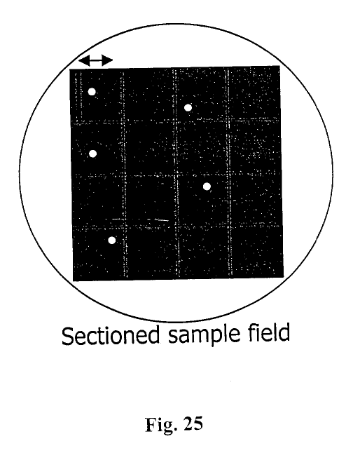

FIG. 25 shows a top view of a sectioned sample field. The sample field,as shown has been sectioned into 16 sub-regions, arranged as a 4×4 array. The various sections may be separately interrogated. Generally, commercially available optics may be utilized to generate lines having a size of about 200 microns×15 microns. While not limited to the specifics stated here, the width of the line is typically on the order of the size of the cell or particle to be interrogated. By utilizing the sectioned sample field of FIG. 25, a relatively shorter line may be utilized, with the result that the line is more linear.

FIG. 26 shows a top view of a multiple line separation system. Six lines are shown having a timeline separation. Generally, the line separation is chosen such that the presence of the nearest neighbor line has an insubstantial effect on the neighboring particles.

The apparatus and methods of these inventions utilize optical forces, either alone or in combination with additional forces, to characterize, identify, select and/or sort material based upon different properties or attributes of the particles. The optical profiles may be static, though vary with position, or dynamic. When dynamic, both the gradient fields as well as the scattering forces may be made to move relative to the particle, medium containing the particle, the support structure containing the particle and the medium. When using a moving optical gradient field, the motion may be at a constant velocity (speed and direction), or may vary in a linear or non-linear manner.

The optical forces may be used in conjunction with other forces. Generally, the optical forces do not interfere or conflict with the other forces. The additional forces may be magnetic forces, such as static magnetic forces as generated by a permanent magnet, or dynamic magnetic forces. Additional electric forces may be static, such as electrostatic forces, or may be dynamic, such as when subject to alternating electric fields. The various frequency ranges of alternating electromagnetic fields are generally termed as follows: DC is frequencies much less than 1 Hz, audio frequencies are from 1 Hz to 50 kHz, radio frequencies are from 50 kHz to 2 GHz, microwave frequencies are from 1 GHz to 200 GHz, infrared (IR) is from 20 GHz to 400 THz, visible is from 400 THz to 800 THz, ultraviolet (UV) is from 800 THz to 50 PHz, x-ray is from 5 PHz to 20 EHz and gamma rays are from 5 EHz and higher (see, e.g., Physics Vade Mecum). .) The frequency ranges overlap, and the boundaries are sometimes defined slightly differently, but the ranges are always substantially the same. Dielectrophoretic forces are generated by alternating fields generally being in the single Hz to 10 MHz range. For the sake of completeness, we note that dielectrophoretic forces are more electrostatic in nature, whereas optophoretic forces are electromagnetic in nature (that is, comparing the frequency ranges is not meant to imply that they differ only in their frequency.) Gravitational forces may be used in conjunction with optical forces. By configuring the orientation of the apparatus, the forces of gravity may be used to affect the actions of the particle. For example, a channel may be disposed in a vertical direction so as to provide a downward force on a particle, such as where an optical force in the upward direction has been generated. The force of gravity takes into consideration the buoyancy of the particle. When a channel is disposed in the horizontal direction, other forces, e.g., frictional forces, may be present. Fluidic forces (or Fluidics) may be advantageously utilized with optical forces. By utilizing an optical force to effect initial particle separation, a fluidic force may be utilized as the mechanism for further separating the particles. As yet another additional force, other optical forces may be applied against the particle. Any or all of the aforementioned additional forces may be used singly or in combination. Additionally, the forces may be utilized serially or may be applied simultaneously.

FIGS. 14 and 15 show sorting of particles or objects from a one-dimensional source. As shown in FIG. 14, particles 320 progress in a generally downward direction from a source in the direction of the arrow labeled particle flow. At junction 322, and possibly additionally before the junction 322, the particles are subject to an optical separation force. Those particles having a different response property, such as a different dielectric constant, may be separated from the line of particles resulting in the separated particles 326. Those particles which are not separated continue on as the particles 324. FIG. 15 shows optical cell sorting from a one-dimensional source. Cells 330 move in a fluid flow in a direction from top to bottom as shown by the arrow. The cells 330 are subject to an optical force in the region of junction 332. Selected cells 336 are deviated from the path of the original fluid flow. The remaining particles 334 continue on in the same direction as the original fluid flow. It will be appreciated that the term “selected” or “non-selected” or similar terminology as used herein is meant to be illustrative, and not intended to be limiting.

The techniques of this invention may be utilized in a non-guided, i.e., homogeneous, environment, or in a guided environment. A guided environment may optionally include structures such as channels, including microchannels, reservoirs, switches, disposal regions or other vesicles. The surfaces of the systems may be uniform, or may be heterogeneous.

A computerized workstation may-include a miniaturized sample station with active fluidics, an optical platform containing a laser (e.g., a near infrared laser for biological applications) and necessary system hardware for data analysis and interpretation. The system may include real-time analysis and testing under full computer control.

The inventions herein may be used alone, or with other methods of cell separation. Current methods for cell separation and analysis include flow cytometry, density gradients, antibody panning, magnetic activated cell sorting (“MACS™”), microscopy, dielectrophoresis and various physiological and biochemical assays. MACS separations work only with small cell populations and do not achieve the purity of flow cytometry. Flow cytometry, otherwise known as Fluorescent Activated Cell Sorting (“FACS™”) requires labeling.

In yet another aspect, the systems of the present invention may optionally include sample preparation steps and structure for performing them. For example, sample preparation may include a preliminary step of obtaining uniform size, e.g., radius, particles for subsequent optical sorting. The systems may optionally include disposable components. For example, the channel structures described may be formed in separable, disposable plates. The disposable component would be adapted for use in a larger system that would typically include control electronics, optical components and the control system. The fluidic system may be included in part in the disposable component, as well as in the non-disposable system components.

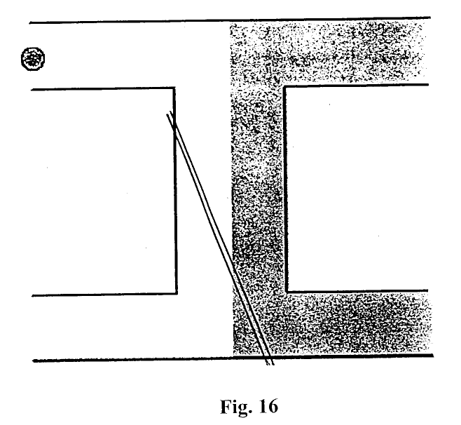

FIG. 16 shows a plan view of a microfluidic system for sorting particles by means of a static optical gradient. As shown, a generally “H” shaped microfluidic structure is depicted Other microfluidic arrangements may be utilized to implement the instant invention, though the “H” structure has desirable performance. The structure includes a first inlet and a second inlet. The inlets are fluidically coupled, and are connected to a separation region. A first outlet and a second outlet are coupled downstream of the separation region. As shown, the first inlet is at the upper left of the Figure, and the first outlet is at the lower left of the Figure. First fluid flow, preferably laminar fluid flow, proceeds from the first inlet, through the left hand side of the separation region, and out through the first outlet. Correspondingly, second fluid flow, preferably laminar fluid flow, proceeds from the second inlet, through the right hand side of the separation region, and out through the second outlet.

The first fluid and second fluid may be the same type of carrier fluid, e.g. water, but would be expected to have different particle constitutions. For example, the first fluid may contain the particle population to be sorted, and the second fluid, as entering in the second inlet, contains no particles. While it is stated that there is a first and second fluid, and that the fluid flow is preferably laminar, there may be some admixing or diffusion between the two fluids.

An optical gradient is disposed diagonally across at least a portion of the separation region. By diagonally it is meant that the optical gradient has at least a component in the direction parallel to the bulk fluid flow. Preferably, the optical gradient crosses the entirety of the first fluid in the separation region. Additionally, the optical gradient may cross some or all of the second fluid. As shown, the optical gradient intersects the first fluid at the wall of the separation region containing the first fluid, and at the other end, intersects the wall which continues into the second outlet.

In operation, a particle or particles enter the first inlet in a first fluid and flow through the generally left hand portion of the separation region. The second fluid is flowing through the generally right hand portion of the separation region. At some point, the particle in the first fluid will arrive at the optical gradient. If the particle interacts sufficiently with the gradient, the particle will be displace from the first fluid to the second fluid. Once in the flow of the second fluid, the particle will flow out through the second outlet. If the particle does not interact sufficiently with the optical gradient, it will continue to flow in the first fluid, and flow out of the first outlet. In this way, particles that interact more strongly with the optical gradient may be displaced from the first fluid to the second fluid, and thereby removed from the system by different outlets.

The system may be termed a static optical gradient. The optical gradient may be static relative to the physical microfluidic structure. Relative movement of the particle and the optical gradient is achieved through the flowing of the particle in the fluid. By providing relative motion between the particle and the optical gradient, a differential force may be imparted on particles based on their optophoretic properties.

Methods for Reducing or Modifying Forces

The system and methods may include various techniques for reducing or otherwise modifying forces. Certain forces may be desirable in certain applications, but undesirable in other applications. By selecting the technique to reduce or minimize the undesired forces, the desired forces may more efficiently, sensitively and specifically sort or identify the desired particles or conditions. Brownian motion of particles may be an undesired condition for certain applications. Cooling of the system may result in a reduced amount of Brownian motion. The system itself may be cooled, or the fluidic medium may be cooled.

Yet another force which may be undesired in certain applications is friction or other form of sticking force. If surface effects are to be minimized, various techniques may be utilized. For example, a counterpropagating beam arrangement may be utilized to capture particles and to remove them from contact with undesired surfaces.

Yet other techniques exist for addressing friction, stiction, electrostatic and other surface interactions which may interfere with the mobility of cells and/or particles. For example, surfaces may be treated, such as through the use of covalent or non-covalent chemistries, which may moderate the frictional and/or adhesion forces. Surfaces may be pretreated to provide better starting surfaces. Such pretreatments may include plasma etching and cleaning, solvent washes and pH washes, either singly or in combination. Surfaces may also be functionalized with agents which inhibit or minimize frictional and adhesive forces. Single or multi-step, multi-layer chemistries may be utilized. By way of example, a fluorosilane may be used in a single layer arrangement which renders the surface hydrophobic. A two-step, two-layer chemistry may be, for example, aminopropylsilane followed by carboxy-PEG. Teflon formal coating reagents such as CYTOP™ or Parylene™ can also be used. Certain coatings may have the additional benefit of reducing surface irregularities. Functional groups may, in certain cases, be introduced into the substrate itself. For example, a polymeric substrate may include functional monomers. Further, surfaces may be derivitized to provide a surface which is responsive to other triggers. For example, a derivatized surface may be responsive to external forces, such as an electric field. Alternatively, surfaces may be derivatized such that they selectively bind via affinity or other interactions.

Yet another technique for reducing surface interactions is to utilize a biphasic medium where the cells or particles are kept at the interface. Such aqueous polymer solutions, such as PEG-dextran partition into two phases. If the cells partitioned preferentially into one of the layers, then under an optical gradient the cells would be effectively floating at the interface.

Methods for Enhancing or Changing the Dielectric Constant

Optionally, the particles to be subject to the apparatus and methods of these inventions may be either labeled or unlabeled. If labeled, the label would typically be one which changes or contributes to the dielectric constant of the particle or new particle (i.e., the initial particle and the label will act as one new particle). For example, a gold label or a diamond label would effectively change most typical dielectric constants of particles.

Yet other systems may include an expressible change in dielectric constant. For example, a genetic sequence may exist, or be modified to contain, an expressible protein or other material which when expressed changes the dielectric constant of the cell or system. Another way to tune the dielectric constant of the medium is to have a single medium in a fluidic chamber where the dielectric constant can be changed by changing the temperature, applying an electric field, applying an optical field, etc. Other examples would be to dope the medium with a highly birefringent molecule such as a water-soluble liquid crystal, nanoparticles, quantum dots, etc. In the case of birefringent molecules, the index of refraction that the optical beam will see can be altered by changing the amplitude and direction of an electric field.

Methods for Increasing Sensitivity

Maximizing the force on a particle for a given intensity gradient suggests that the difference in dielectric constant between the particle and medium should be maximized. However, when sensitivity is required in an application, the medium should be selected such that the dielectric constant of the medium is close to the dielectric constant of the particle or particles to be sorted. By way of example, if the particle population to be sorted has dielectric constants ranging from 1.25 to 1.3, it would be desirable to choose a dielectric constant which is close to (or even within) that range. For cells, a typical range of dielectric constants would be from 1.8 to 2.1. By close, a dielectric constant within 10% or, more particularly, within 5%, would be advantageous. While the absolute value of the magnitude of the force on the particle population may be less than in the case where the dielectric constant differs markedly from the dielectric constant of the medium, the difference in resulting motion of the particles may be larger when the dielectric constant of the medium is close to the range of dielectric constants of the particles in the population. While utilizing the increased sensitivity of this technique at the outset, once the separation begins, the force may be increased by changing the dielectric constant of the medium to a more substantial difference from the dielectric constants of the particle or particle collection. As indicated, it is possible to choose the dielectric constant of the medium to be within the range of dielectric constants of the particle population. In that instance, particles having a dielectric constant above the dielectric constant of the medium will feel a force in one direction, whereas those particles having a dielectric constant less than the dielectric constant of the medium will feel a force moving in the opposite direction.

Static Systems

FIG. 19A shows a system for the measurement of dielectric constants of particles. A particle 558 having a dielectric constant may be subject to different media having different dielectric constants. As shown, a first vessel 550, a second vessel 552, and so on through an end vessel 554 contain a medium having different dielectric constants ε₁ ε₂, . . . εₙ, respectively. By illuminating the particle 558 with an optical gradient force 556, and observing the motion, the dielectric constant of the particle may be determined. If the dielectric constant of the medium is equal to the dielectric constant of the particle then no force is imposed by the optical illumination 556. In contrast, if there is a difference between the dielectric constant of the particle and the dielectric constant of the medium, an optical force will be imposed on the particle by the optical illumination 556. Different dielectric constant media may be supplied as shown in FIG. 19A, namely, where a plurality a vessels 550, 552 . . . 554 are provided. Alternately, a particle may be subject to a varying dielectric constant over time, such as through use of a titration system. In on implementation, the titration may be accomplished in a tube containing the particle by varying the dielectric constant of the fluid over time, such as by mixing fluids having different dielectric constants, preferably at the inlet to the tube, or by providing a varying dielectric constant profile, such as a step profile. Additionally, the dielectric constant of a particle may be approximated by interpolation, such as where two or more data points are obtained regarding the force on the particle in different media, and then the expected dielectric constant in which no force is present may be determined.

FIG. 19B shows a static system in which separation may occur. A light pattern 560 illuminates first particle 562 and second particle 564. If the dielectric constant of the first particle 562 is less than the dielectric constant of the medium, then the particle moves toward an area of lower intensity. In contrast, if the second particle 564 has a dielectric constant which is greater than the dielectric constant of the medium, the particle will move toward the region of higher intensity. As a result, the first particle 562 and second particle 564 are subject to forces in opposite directions. Given the proximity shown, they would move away from one another.

The first setup is a moving fringe workstation for Optophoresis experiments. A high power, 2.5 watt, Nd—YAG laser (A) is the near IR, 1064 nm wavelength, light source. The fringe pattern is produced by directing the collimated laser beam from the mirror (1) through the Michelson interferometer formed by the prism beam splitter (2) and the carefully aligned mirrors (3). A variable phase retarder (4) causes the fringe pattern to continuously move. This fringe pattern is directed by the periscope (5) through the telescope (5a) and (5b) to size the pattern to fill the back focal plane of the microscope objective, and then is directed by the dichroic beam splitter (6) through a 20×microscope objective (7) to produce an image of the moving fringe pattern in the fluidic chamber holding the sample to be sorted. A second, 60×microscope objective (8) images the flow cell onto a CCD camera to provide visualization of the sorting experiments. A fiber-optic illuminator (9) provides illumination, through the dichroic beam splitter (6), for the sample in the fluidic chamber. The fluidic chamber is positioned between the two microscope objectives by means of an XYZ-translation stage.

It will be appreciated by those skilled in the art that there are any number of additional or different components which may be included. For example, additional mirrors or other optical routing components may be used to ‘steer’ the beam where required. Various optical components for expanding or collimating the beam may be used, as needed. In the set-up implementing FIG. 5, the laser used additional mirrors to steer the laser beam into the spatial filter, which that produced a well collimated Gaussian beam that is then guided to the Michelson interferometer.

The second setup is a workstation for measuring and comparing the dielectric properties of cells and particles at near IR optical frequencies, using a 600 mW, ultra-low noise Nd—YAG laser (B) as a light source. The remainder of the optical setup is similar to the moving fringe workstation, except there is no interferometer to produce moving fringes. Instead a single, partially focused illumination spot is imaged within the fluidic chamber. The interaction of cells with this illumination field provides a measurement of the dielectric constant of the cells at near IR optical frequencies.

Exemplary Applications

Novel Technology For Use in Systems Biology

The methods and apparatus herein permit a robust cell analysis system suitable for use in systems biology in pharmaceutical and life sciences research. This system may be manufactured using higher performance, lower cost optical devices in the system. A fully integrated systems biology, cell analysis workstation is suitable for use in drug discovery, toxicity and life science research.

These systems may utilize advanced optical technologies to revolutionize the drug discovery process and cellular characterization, separation and analysis by integrating Optophoresis technology into devices for the rapid identification, selection and sorting of specific cells based on their innate properties, including their innate optical dielectric properties. In addition, since the technology is based on the recognition of such innate properties, labels are not required, greatly simplifying and accelerating the testing process. The lasers employed are preferably in the biologically-compatible infrared wavelengths, allowing precise cell characterization and manipulation with little or no effect on the cell itself. The technology is suited to the post-genomics era, where the interaction of the cell's molecular design/make-up (DNA, RNA and proteins) and the specific cellular changes (growth, differentiation, tissue formation and death) are of critical importance to the basic understanding of health and disease.

The Optophoresis technology changes the nature of cell-based assays. Applications would include all methods of cellular characterization and sorting. The technology also offers diverse applications in the areas of molecular and cellular physiology. Optophoresis technology addresses fundamental properties of the cell itself, including its optical dielectric properties. The optophoretic properties of the cell change from cell type to cell type, and in response to external stimuli. These properties are reflective of the overall physiologic status of the cell. Active cells have dielectric properties that are different from resting cells of the same type. Cancer cells have different optophoretic properties than their normal counterparts. These cellular properties can also be used effectively in drug discovery and pharmaceutical research, since nearly all drugs are targeted ultimately to have direct effects on cells themselves. In other words, drugs designed to effect specific molecular targets will ultimately manifest their effects on cellular properties as they change the net dielectric charge of the cell. Therefore, rapid screening of cells for drug activity or toxicity is an application of the technology, and may be referred to as High Throughput Biology. Other main applications include drug discovery and pharmaceutical research.

The Human Genome Project and other associated genome programs will provide enormous demand for improved drug development and screening technologies. Sophisticated cellular approaches will be needed for cost-effective and functional screening of new drug targets. Likewise, information from the genome projects will create demand for improved methods of tissue and organ engineering, each requiring access to well characterized cellular materials. Moreover, optical technology from the information and telecommunications industry will provide the system hardware for improved optical cell selection and sorting. The price/performance ratios for high powered near infrared and infrared lasers originally developed for telecommunications applications continue to improve significantly. In addition, solid-state diode lasers may be used having a variety of new wavelengths, with typically much higher power output than older versions.

A computerized Workstation may be composed of a miniaturized sample station with active fluidics, an optical platform containing a near infrared laser and necessary system hardware for data analysis and interpretation. The system includes real-time analysis and testing under full computer control. Principal applications of the technology include cell characterization, monitoring, enrichment, and selection, particularly for identifying and selecting distinct cells from complex backgrounds.

Biological Applications

Importantly, unlabelled, physiologically normal, intact test cells will be employed in the system. The sample is quickly analyzed, with the cells classified and sorted by the optical field, thereby allowing characterization of drug response and identify toxicity or other measures of drug efficacy. Characterizing the cellular optophoretic properties uniquely associated with various drug testing outcomes and disease states is a part of this invention. Identification of these novel parameters constitutes useful information.

An integrated system may, in various aspects, permit: the identification, selection and separation of cells without the use of labels and without damaging the cells; perform complex cell analysis and separation tasks with ease and efficiency; observe cells in real time as they are being tested and manipulated; establish custom cell sorting protocols for later use; isolate rare cells from complex backgrounds; purify and enrich rare cells (e.g. stem cells, fragile cells, tumor cells); more easily link cell phenotype to genotype; study cell-cell interactions under precise and optical control; and control sample processing and analysis from start to finish.

The technology offers a unique and valuable approach to building cellular arrays that could miniaturize current assays, increase throughput and decrease unit costs. Single cell (or small groups of cells) based assays will allow miniaturization; and could allow more detailed study of cell function and their response to drugs and other stimuli. This would permit cellular arrays or cell chips to perform parallel high-throughput processing of single cell assays. It could also permit the standardization of cell chip fabrication, yielding a more efficient method for creation of cell chips applicable to a variety of different cells types.

Mammalian cell culture is one of the key areas in both research (e.g., discovery of new cell-produced compounds and creation of new cell lines capable of producing specific proteins) and development (e.g., developing monoclonal cell lines capable of producing highly specific proteins for further research and testing). Mammalian cell culture is also a key technology for the production of new biopharmaceuticals on a commercial scale.

Once researchers have identified drug targets, compounds or vaccines, mammalian cell culture is an important technology for the production of quantities necessary for further research and development.

Optical cell characterization, sorting and analysis technologies could be useful in selecting and separating lines of mammalian cells according to whether they produce a new protein or biopharmaceutical compound and according to the relative yield of the protein or compound. Cell yield is a key factor in determining the capacity required to produce commercial quantities of a new biotechnology drug.

To this end, optical interrogation methods can be used in biopharmaceutical monitoring and quality control applications. Many pharmaceutical compounds such as active proteins are produced by living cells contained in a bioreactor. Optophoresis can be employed to monitor one or more parameters within the bioreactor to ensure optimal expression of the pharmaceutical compound of interest. For example, optical interrogation can be used to monitor and quantify the distribution of cells contained within the bioreactor based on their relative protein expression levels. Other parameters indicative of cell health and expression may be monitored using Optophoretic methods.

Optical interrogation methods can also be used in cellular enrichment applications. When pharmaceutical compounds are produced in bioreactors, it is often preferable to retain only those cells that have a particular biological property. One particular biological property of interest is the relative level of protein expression. In this regard, it is preferable to retain only those cells with high levels of protein expression. The cells with low levels of protein expression can be removed and discarded. This method can advantageously be integrated into bioreactor designs to recycle the cells having high levels of protein expression back to the bioreactor.

Potential Applications

We turn now to more specific discussions of applications. First, we address separation applications, and second, address monitoring applications.

Separation Applications

White cells from red cells. In some instances, such as in the case of transfusions, white cells need to be separated from red cells prior to transfusion for better tolerance and to decrease infection risks. In other contexts, it often important to separate out red cells in order to obtain enriched populations of white cells for subsequent analysis or manipulation. Optophoresis can allow the separation of white cells from red blood cells for use in applications where a single or enriched population is desired.

Reticulocytes from mature red blood cells. Reticulocytes are immature red blood cells normally found at very low levels. Increased levels of reticulocytes, however, can be indicators of disease states. Optophoresis may be use to separate and determine the levels of reticulocytes from whole blood in order diagnose a potential disease condition.

Clinical Care Applications, e.g., Fetal stem cells from maternal circulation. Optophoresis is a potential tool that may allow the successful isolation of fetal cells from maternal blood. In this regard, Optophoresis may enable fetal DNA to be obtained in a non-invasive manner. Fetal cells obtained from a maternal blood sample can undergo further analysis to permit the diagnosis of genetic disorders such as, for example, Down's Syndrome. Optophoretic separation and concentration of fetal cells would permit the prenatal diagnosis of a variety of genetic abnormalities from a single maternal blood sample.

Clinical Care Applications, e.g., Stem Cell Isolation. Optophoresis may be used as a tool to isolate and purify stem cells from stem cell grafts for transplantation, i.e., to remove T-cells in allogenic grafts (where the donor and the recipient are not the same person) and cancer cells in autologous grafts (where the donor and the recipient are the same person). Current stem cell separation technologies suffer from several drawbacks, including, low recovery yields.

Tumor cells from blood. Minimal Residual Disease (MRD) Testing Optophoresis technology may address some of the key unmet needs for better cancer screening, including accurate, reproducible and standardized techniques that can detect, quantify and characterize disseminated cancer cells; highly specific and sensitive immunocytological techniques; faster speed of cell sorting; and techniques that can characterize and isolate viable cancer cells for further analysis.

Cancer cells are typically found in low numbers circulating in the blood of patients, particularly when metastasis has occurred. The presence of tumor cells in the blood can be used for a diagnosis of cancer, or to follow the success or failure of various treatment protocols. Optophoresis provides a potential means of separating our enough cells to detect and thus accurately diagnose the patient.

Another potential application for Optophoresis is in the removal of tumor cells from blood or stem cell products prior to use in autologous transplants for cancer patients.

Fetal stem cells from cord blood. The umbilical cord from a newborn generally contains blood which is rich in stem cells. The cord blood material is usually discarded at birth but can, however, be harvested for future use such as, for example, autologous or allogenic stem cell replacement. Enrichment of the cord blood stem cells by Optophoresis may allow for a smaller amount of material to be stored, which could be more easily given back to the patient or another host.

Adult stem cells from liver, neural tissue, bone marrow, and the Like. Many mature tissues have small subpopulations of immortal stem cells which may be manipulated ex vivo and then can be reintroduced into a patient in order to repopulate a damaged tissue. Optophoresis may be used to purify these extremely rare adult stem cells so that they may be used for cell therapy applications.

Islet cells from pancreas. It may be possible to increase insulin production in diabetic patients by transplanting the insulin producing beta islet cells from a healthy pancreas into the diabetic person. The islet cells, however, make up only a small fraction of the total donor pancreas. Optophoresis may provide a method to separate and enrich the islet cells for transplantation.

Activated B or T cells. During an immune response either T or B white cell subsets which target a specific antigen become active These specific activated cells may be required as separate components for use in ex vivo expansion to then be applied as immunotherapy products or to be disposed of, since activated B or T cells can cause unwanted immune reactions in a patient such as organ rejection, or autoimmune diseases such as lupus or rheumatoid arthritis. Optophoresis may provide a method to obtain activated cells either to enrich and give back to a patient. Alternatively, Optophoresis may allow the enrichment of cells that are causing pathological destruction so that they can be discarded.

Dendritic cells. Dendritic cells are a subset of white blood cells which are critical to establishing a T-cell mediated immune response. Dendritic cells can be harvested and used ex vivo in conjunction with an appropriate antigen to produce a specific activated T cell response. Optophoresis may allow isolation of large numbers of dendritic cells for such work.

HPRT-cells. Hypoxanthine-guanine phosphoribosyltransferase (HPRT) is an enzyme which exits in many cells of the blood and is involved in the nucleoside scavenging pathway. Persons who have high mutation rates due to either endogenous genetic mutations or exogenous exposure to mutagens can be screened for HPRT lacking cells (HPRT-) which indicate that a mutation has occurred in this gene. Optophoresis following screening by compounds which go through the HPRT system may be used to select HPRT-cells and quantitate their numbers.

Viable or mobile sperm cells. In significant percentage of infertility cases, infertility is attributed to in whole or in part to factors associated with males. Semen analysis is currently performed using a variety of tests and is based on a number of parameters including count, volume, pH, viscosity, motility and morphology. At present, semen analysis is a subjective and manual process, the results of which do not always clearly indicate if the male is contributing to the couple's infertility. Sperm selection is typically accomplished using either gradient centrifugation to isolate motile sperm used in In Utero Insemination (IUI) and In Vitro Fertilization (IVF) or visual inspection and selection to isolate morphologically correct sperm used in IVF and Intracytoplasmic Sperm Injection (ICSI).

One of the reasons for male infertility is the low counts of viable and/or mobile sperm cells. It is possible that viable and/or mobile sperm cells may be selected using Optophoresis and therefore enrich their numbers. Consequently, it may be possible to increase the chances of fertilization using the enriched sperm cells. It is also possible that Optophoresis may be used to select X from Y bearing sperm and vice versa, which would then be used selectively to induce pregnancies in animal applications where one sex of animal is vastly preferred for economic reasons (e.g., it is preferable that dairy cows be female, while it is preferable for meat producing cattle to be male).

Liposomes loaded with various compounds. A more recent mode of therapeutic drug delivery relies on the use of liposomes as drug delivery vehicle. Optophoresis can be employed to separate liposomes containing different levels of drug to thereby select those liposomes in which the drugs are most concentrated. In addition, Optophoresis can be used to select certain cells or groups of cells based on their uptake of drug-containing liposomes.

Tissue Engineering, e.g., Cartilage precursors from fat cells. Tissue engineering involves the use of living cells to develop biological substitutes for tissue replacements which can be used in place of traditional synthetic implants. Researches have recently reported that cells found in human adipose tissue can be used ex vivo to generate cartilage which can be used as a transplant material to repair damage in human joints. Optophoresis may be used to purify the cartilage forming cells from the other cells in adipose tissue for ex vivo expansion and eventual tissue engineering therapy.

Nanomanipulation of small numbers of cells. Recent miniaturization of many lab processes have resulted in many lab analyses being put onto smaller and smaller platforms, evolving towards a “lab-on-a-chip” approach While manipulation of biomolecules in solution has become routine in such environments, manipulation of small numbers of cells in microchannel and other nano-devices has not been widely achieved. Optophoresis may allow cells to be moved in microchannels and directed into the region(s) with the appropriate processes on the chip.

Cellular organelles; mitochondria, nucleus, ER, microsomes. The internal constituents of a cell consists of the cytoplasm and many organelles such as the mitochondria, nucleus, etc. Changes in the numbers or physical features of these organelles can be used to monitor changes in the physiology of the cell itself. Optophoresis can allow cells to be selected and enriched which have particular types, morphologies or numbers of a particular organelle.

Cow reticulocytes for BSE assays. It is known that a cellular component of the reticulocyte, EDRF, is found at elevated levels in the reticulocytes of cows infected with BSE (bovine spongiform encephalopathy). Reticulocytes are generally found at low levels in the blood and therefore the use of Optophoresis may allow their enrichment and would increase the accuracy of diagnostic tests based on the quantitation of the EDRF mRNA or protein.

Monitoring

Growing/dividing cells vs. resting cells. Cells may be stimulated to grow by various growth factors or growth conditions. Most current assays for cell growth require the addition of external labeling reagents and/or significant time in culture before cell growth can be demonstrated. By using Optophoresis, however, cells which have begun to divide can be identified, providing a rapid method for calculating how much of a given cell population is in the growth phase. In addition, cells in different parts of the cell cycle have different optical properties and these may be used to either sort cells based on where in the cycle they are as well as to determine what fraction of the total cell population is in each stage of the cell cycle.

Apoptotic cells. Cells which are undergoing programmed cell death or apoptosis can be used to identify specific drugs or other phenomenon which lead to this event. Optophoresis may be used to identify which cells are undergoing apoptosis and this knowledge can then be used to screen novel molecules or cell conditions or interactions which promote apoptosis.

Cells with membrane channels open; change in membrane potentials. The outer membrane of many types of cells contain channels which facilitate the passage of ions and small molecules into and out of the cell. Movement of such molecules can lead to changes in electrical potential, changes in levels of second messengers, etc. Identifying these changes can be useful in drug screening for compounds which modulate membrane channel activity. Optophoresis may be used to determine whether and to what extent membrane channels are open such as, for example, when membrane channels are being perturbed by exogenous compounds.

Live vs. dead cells. Many applications exist which require the identification and quantitation of living and dead cells. Optophoresis provides a quick method of identifying and separating dead cells from living cells. This technique can be used to identify, quantify, as well as sort live/dead cells for all types of cells, including mammalian cells.

Virally infected cells. Optophoresis is able to identify, detect, and separate cells that are infected with viruses. In addition, Optophoresis can be used to differentiate cells or groups of cells based on their relative levels of infection. Optophoresis has been found to detect the effects of infection prior to other conventional techniques (i.e., fluorescence labeling).

Cells with abnormal nucleus or elevated DNA content. It is generally known that tumor cells are able to be identified by the presence of excess DNA, resulting in an abnormal size and/or shape to the cell's nucleus. Optophoresis tuned to the nuclear content of a cell population with abnormal amounts of DNA and/or nuclear structure may be identified and separated. This information can then be used as a diagnostic or prognostic indicator for cancer.

Cells decorated with antibodies. A large selection of commercially available antibodies exists which have specificities to cellular markers which define unique proteins and/or types of cells. Many diagnostic applications rely on the characterization of cell types by identifying what antibodies bind to their surface. Optophoresis may be used to detect when a cell has a specific antibody or antibodies bound to it. Optophoresis may also be used to discriminate between different cells or cell populations having varying amounts of antibody bound to their surface.

Cells with bound ligands, peptides, growth factors. Many compounds and proteins bind to receptors on the surface of specific cell types. Such ligands may then cause changes inside the cell. Many drug screens look for such interactions. Optophoresis may be used to monitor the binding of exogenous large and small molecules to the outside of the cell, as well as measurement of physiological changes inside the cell as a result of binding.

Bacteria for viability after antibiotic exposure. Bacteria are often tested for sensitivity to a spectrum of antibiotics in order to determine the appropriate therapy. Optophoresis can be used to monitor bacterial cells for viability and for cessation of growth following antibiotic exposure. In this regard, Optophoresis can be used to screen bacteria for drug sensitivity.

Drug screening on the NCI 60 panel. A panel of 60 tumor cell lines has been established by the National Cancer Institute as a screening tool to determine compounds which may have properties favorable to use as chemotherapeutic agents. Optophoresis may be used to array all 60 lines and then to expose them with known and novel chemical compounds to determine their potential as possible drug candidates.

Cells for cytoskeletal changes. The cytoskeleton is a complex of structural proteins which keeps the internal structure of the cell intact. Many drugs such as taxol, vincristine, etc. as well as other external stimuli such as temperature are known to cause the disruption and/or breakdown of the cytoskeleton. Optophoresis provides a method to monitor cells or populations of cells for perturbations in the cytoskeleton in response to an applied chemical compound or other external stimulus.

Beads with compounds bound to them, to measure interactions with the cell surface or with other beads. The interactions of microspheres with cells or other compounds have been used in a number of in vitro diagnostic applications. Chemical compounds may be attached to beads and the interactions of the beads with cells may be monitored using Optophoresis. Optophoresis can also monitor the interaction of the beads with the applied compounds.

Progenitor cell/colony forming assays. Progenitors are cells of a given tissue which can give rise to large numbers of more mature cells of that same tissue. A typical assay for measuring progenitor cells is to allow these cells to remain in culture and to count how many colonies of the appropriate mature cell type form in a given amount of time. This type of assay is, however, slow and cumbersome. Optophoresis may be employed to monitor the growth of a single cell. In this regard, progenitor proliferation can be measured on a nano-scale and results should be obtained within a much shorter amount of time.

Dose limiting toxicity screening. Almost all compounds are toxic at some level, and the specific levels of toxicity of compounds are identified by measuring at what concentration they kill living cells. Optophoresis can be used to identify the concentration at which a particular chemical compound kills living cells. Generally, this is performed by slowly increasing the concentration of the chemical compound and optophoretically interrogating the cells to determine when the concentration reaches toxic levels.

Monitor lipid composition/membrane fluidity in cells. The membranes of all cells are composed of lipids which must maintain both the proper degree of membrane fluidity at the same time that they maintain basic cell membrane integrity. Optophoresis may be able to discriminate cells based on their lipid composition and/or membrane fluidity. In addition, Optophoresis can be used to provide information on compounds and conditions which affect membrane fluidity.

Measure clotting/platelet aggregation. Components found in the blood such as platelets and clotting proteins are needed to facilitate blood clot formation. Currently, clotting measurements are often used in order to measure disease states or to assess basic blood physiology. Optophoresis may provide information on platelet aggregation and clot formation.

Biological Separation Experiments

Certain of the data reported herein were generated with the following setup. Optical gradient fields were generated using a Michelson interferometer and either a 150 mW, 812 nm laser (812 system) or a 2.5 W, 1064 nm laser (1064 system). The 812 system used a 100× (1.25 NA) oil immersion lens to focus the fringe pattern and to visualize the sample. The 1064 system used a 20× objective to focus the fringes and a 60× objective to visualize the sample. In general the sample cell was a coated microscope slide and/or coverslip that was sealed with Vaseline. Coverslip spacers controlled the height of the cell at approximately 150 micrometers.

Coating Of Surfaces; Rain-X™, Agarose, CYTOP, Fluorosilane Scattering forces tend to push the particles or cells against the surface of the sample cell. Therefore, a number of surface coatings were evaluated to minimize nonspecific adhesion and frictional forces. Hydrophobic/hydrophilic and covalent/noncovalent surface treatments were evaluated.

Covalent/Hydrophobic Glass slides and coverslips were treated with perfluoro-octyltrichlorosilane (Aldrich, Milwaukee, Wis.) using solution or vapor deposition. Solution deposition was as follows: a 2-5% silane solution in ethanol, incubate 30 minutes at room temperature, rinse 3 times in ethanol and air dry. Vapor deposition involved applying equal volumes of silane and water in separate microcentrifuge tubes and sealing in a vacuum chamber with the substrate to be treated. Heat to 50° C., 15 hrs.

Noncovalent/Hydrophobic—A commercial water repellent containing polysiloxanes, Rain-X, was applied according to the manufacturer's instructions.

A liquid Teflon, CYTOP (CTL-107M, Wilmington, Del.) was spun coated using a microfuge. The CYTOP was diluted to 10% in fluorooctane (v/v) and 50 microliters was pipetted and spun for 5 seconds. This was repeated a second time and then air dried.

Noncovalent/Hydrophilic—Agarose hydrogel coatings were prepared as follows: melt 2% agarose in water, pipette 100 microliters to the substrate, spin for 5 seconds, bake at 37° C. for 30 minutes.

All of the coatings were effective when working with particles. The CYTOP was more effective at preventing adhesion when working with biological cells.

Separation Red Blood Cells vs. Reticulocytes

A reticulocyte control (Retic-Chex) was obtained from Streck Labs. A sample containing 6% reticulocytes was stained for 15 minutes with New Methylene Blue for 15 minutes, a nucleic acid stain that differentially stains the reticulocytes versus the unnucleated red blood cells. The sample was diluted 1/200 in PBS and mounted on a fluorosilane coated slide The 812 system was used to generate optical gradient fields. The fringe period was adjusted to 15 micrometers and was moved at 15 micrometers/second. The reticulocytes were preferentially moved relative to red blood cells.

Separation of White Blood Cells vs. Red Blood Cells

A whole blood control (Para12 Plus) was obtained from Streck Labs. The sample was stained for 15 minutes with New Methylene Blue, a nucleic acid stain that differentially stains the nucleated white blood cells versus the unnucleated red blood cells. The sample was diluted 1/200 in PBS and mounted on a fluorosilane coated slide. The 812 system was used to generate optical gradient fields. The fringe period was adjusted to 15micrometers and was moved at 22 micrometers/second. The white blood cells were moved by the fringes while the red blood cells were not.

Separation of Leukemia vs. Red Blood Cells

One milliliter of the leukemia cell line U937 suspension was pelleted and resuspended in 100 microliters PBS containing 1% BSA. Equal volumes of U937 and a 1/200 dilution of red blood cells were mixed together and 10 microliters was placed on a CYTOP coated slide Separate measurements with moving fringe fields showed that the escape velocity for U937 cells was significantly higher than the escape velocity for red blood cells, 60 and 23 micrometers/second, respectively. The 1064 nm system was used to generate optical gradient fields with a fringe period of approximately 30 micrometers and moving at 45 micrometers/second, an intermediate fringe velocity. As expected the U937 cells move with the fringes and the red blood cells do not. In one embodiment, the moving fringe may be reduced to a single peak. Preferably, the peak is in the form of a line. In operation, a slow sweep (i.e., at less than the escape velocity of the population of particles) is made across the region to be interrogated. This causes the particles to line up. Next, the fringe is moved quickly (i.e., at a speed greater than the escape velocity of at least some of the particle in the population), preferably in the direction opposite the slow sweep. This causes the selective separation of those particles having a higher escape velocity from those having a lower escape velocity. Optionally, the remaining line of particles may then be again interrogated at an intermediate fringe velocity. While this technique has general applicability to all of the applications and systems described herein, it has been successfully implemented for the separation of U937 cells from red blood cells. FIGS. 27A, 27B and 27C show the separation of white blood cells (the larger cells) from red blood cells. The images in FIGS. 27A, B and C correspond to the phases 1, 2 and 3 depicted in FIGS. 21A, B and C.

Sorting of Red Blood Cells vs. White Blood Cells in Microchannels

FIG. 19 shows photographs of sorting of two cell types in a microchannel device. Slide 1 shows a red blood cell and a white blood cell successively entering the moving optical gradient field. Slide 2 shows that white blood cell has been translated down by the action of the moving optical gradient field while the red blood cell has escaped translation. Slides 3 and 4 show that the red blood cell and white blood cell continue to flow into separate channels, completing the sorting.

Sorting of Wild Type/Mutant Yeast Strains

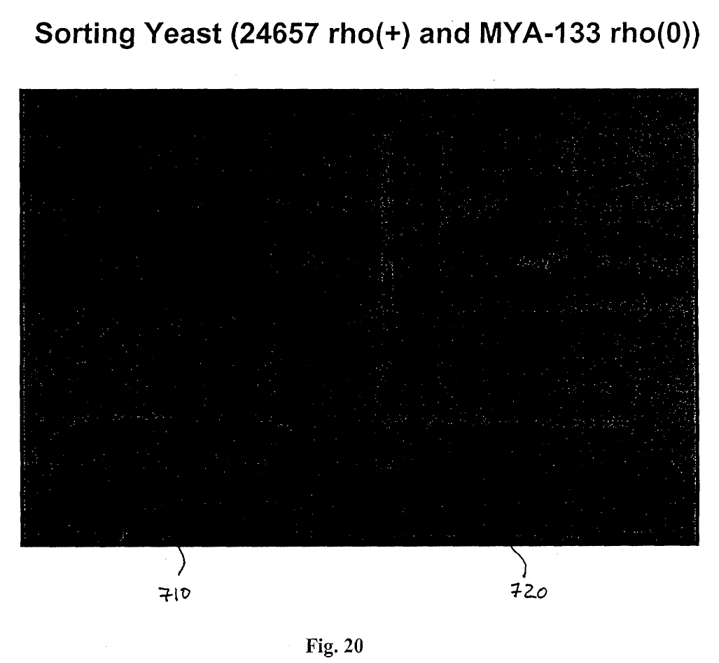

FIG. 20 shows a photograph of a microchannel device 700 used to sort two strains of yeast, 24657 rho+ (wild type) and MYA-1133 rho(0). The difference between the wild type and the mutant yeast strain is that the rho(0) strain lacks mitochondrial DNA. Both strains of yeast pass into the microchannel device 700 in the direction of arrow A due to fluidic flow. The microchannel device 700 has two output channels 710, and 720. A laser line 705 that scans in the direction of arrow B is used to optically interrogate and sort the two strains of yeast. During sorting, the wild type strain passes into output channel 710 while the mutant strain passes into the other output channel 720.

Differential Motion Imaging

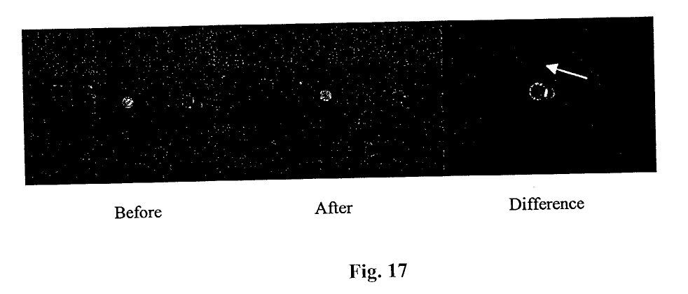

Polystyrene and silica particles were diluted in distilled water. As shown in the photographs of FIG. 17, a “before” image was captured using a CCD camera and Image Pro Express software. A moving optical gradient field generated by the 1064 system was scanned over the particles. Another image (an “After” image) was captured and the “before” image was subtracted. The resultant image (labeled “Difference”) clearly identifies that the polystyrene particle had moved.

Escape Velocities of Different Cell Types

Escape velocities were measured using a gradient field generated by the 1064 system on CYTOP coated coverslips.

| | Escape Velocity |
|---|---|
| Cell Type | (um/sec.) |
| Red Blood Cell | 5.6 +/− 0.4 |
| White Blood Cell | 11.0 +/− 1.8 |
| Chicken Blood (Retic. Model) | 7.3 +/− 1.4 |
| K562 Cells, No Taxol Treatment | 10.0 +/− 0.7 |
| K562 Cells, 26 Hr. Taxol Treatment | 8.2 +/− 0.4 |

K562 Cells: Chronic myelogenous leukemia, lymphoblast

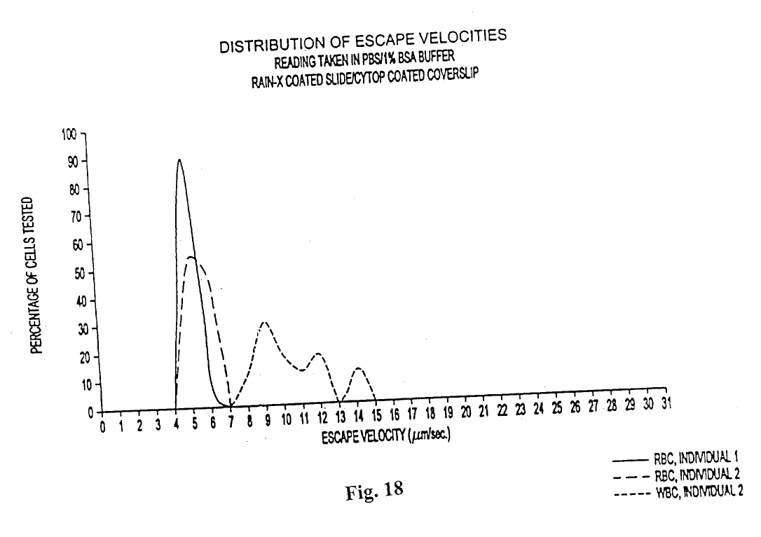

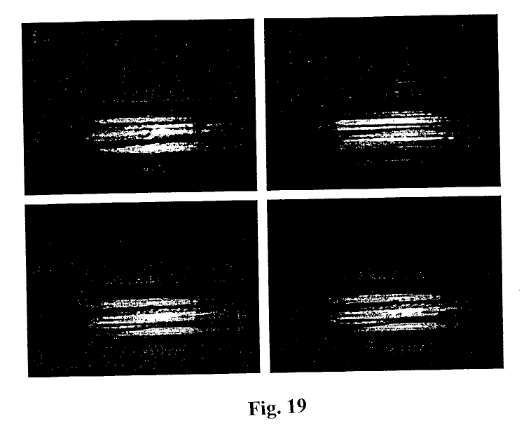

FIG. 18 shows a graph of percent of cells measured as a function of escape velocity (μm/second).

Separation of Drug Treated and Untreated Leukemia Cells

PMA was dissolved in ethanol at a concentration of 5 mg/mL. 3 mls of U937 cells grown in RPMI 1640 media with supplements were removed from the culture flask and 1 ml was placed into each of three eppendorf tubes. Cells from the first tube were pelleted for 4 minutes at 10,000 rpm and resuspended in 250 uL PBS/1% BSA buffer for escape velocity measurements. PMA was added to the remaining two tubes of U937 cells to a final concentration of 5 ug/mL. These tubes were vortexed and placed in a 37° C. water bath for either one hour or six hours. At the end of the time point, the tube was removed, cells were pelleted and then resuspended as described above and escape velocity measurements taken. The cells treated for 6 hours had a significantly higher escape velocity as compared to the untreated cells

Methods for Determining a Biological Property

The methods described herein are useful for determining a biological property of a cell or population of cells using an optical gradient. Any number of biological properties can be determined using an optical gradient. The biological properties can include, for example, whether a cell has been infected by a virus, the degree to which a cell expresses a particular protein, determining at what stage in the cell cycle a particular cell is presently at, whether the cell is affected by the presence of a chemical compound, determining a particular phenotype of the cell, determining whether a ligand is bound to the surface of a cell, determining cytoskeletal changes in the cell, determining whether a cell is decorated with antibodies, detecting the presence or absence of a cellular component (e.g., an organelle or inclusion body), detecting a change in one or more cellular components, and determining the toxicity of chemical compounds. A biological property can also include a physical property of a cell or population of cells. Finally, a biological property can also include a response of a cell or population of cells to an external stimulus such as, for example, a chemical compound.

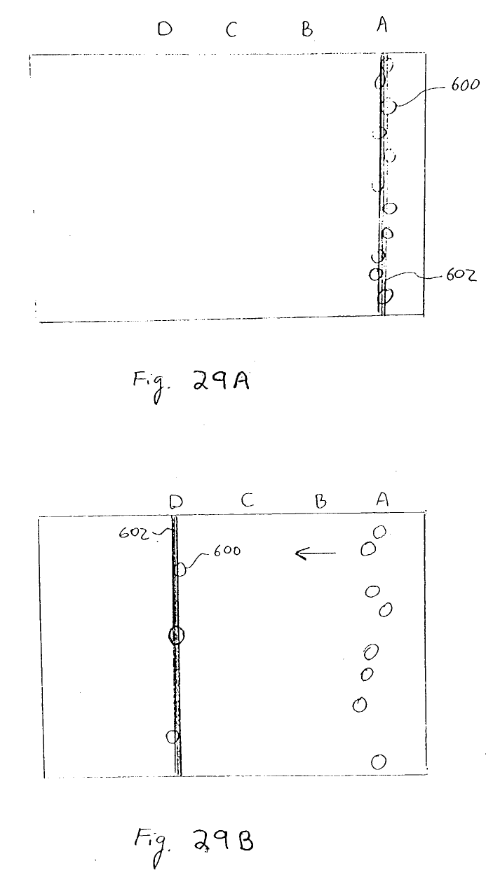

The methods described herein for determining one or more biological properties of a cell or group of cells have applications in a variety of fields. Of particular interest is the use of Optophoresis to monitor operational conditions or parameters of a bioreactor. Many biopharmaceutical products such as, for example, proteins are produced in a bioreactor device such as that disclosed in FIG. 28A. Optophoretic interrogation can be used for quality control purposes, for example, to ensure that the cells contained within the bioreactor are maintained at optimum conditions. The methods can also be used as an early warning detection system that would alert the operator or system if cells contained within the bioreactor were adversely impacted by, for example, an environmental change.

The methods described herein are also useful in cellular enrichment applications. FIG. 28B shows a bioreactor incorporating this feature. In this application, an output stream of a bioreactor is subject to optical interrogation based on one or more biological properties of the cells contained therein. The biological property may include, by way of example, the relative expression level of the biopharmaceutical compound (e.g. a protein). In this example, optical interrogation is used to separate those cells with low expression levels from cells that have higher expression levels. In this application, the cells with low expression levels are separated and discarded while the cells having high expression levels are recycled back to the bioreactor. Cells having high expression levels are desired because more protein can be produced in the bioreactor, thereby increasing production yields.

This later point is particularly important because based on current and projected demand for biotherapeutic proteins, there will soon be a production capacity gap in which the demand for biotherapeutic proteins will exceed the ability of the marketplace to satisfy this growing demand. The present method, however, would permit producers to increase the yield of existing and future production facilities by enriching bioreactors with the highest yielding cells.

The Optophoretic methods described herein are also useful in other monitoring and testing applications. For example, the methods are useful in environmental testing (both airborne and water samples), agricultural testing, food safety testing, as well as biohazard detection and analysis. In this application a sample is provided and moved relative to an optical gradient (or, alternatively, the optical gradient is moved relative to the sample). The relative movement between the sample and the optical gradient allows components of the sample such as, for example, cells, bacteria, yeast, or particulate matter, to be selected, identified, and sorted according to their interaction with the optical gradient.

At its most basic level, the method of determining a biological property of a cell or population of cells using an optical gradient involves moving the cell(s) and the optical gradient relative to each and determining the biological property of the cell(s) as a function of at least the interaction of the cell(s) and the optical gradient. The relative movement can be accomplished be moving the optical gradient relative to the cell, moving the cell relative to the optical gradient, or some combination thereof.

For the experiments and applications discussed below, an optical system of the type shown in FIG. 6 was used to perform the escape velocity and fast scan measurements. Additional description concerning the particular setup of this optical system can be found in paragraph 134 of this Application.

Optical Interrogation—Drug Screening Applications

For drug screening applications, this same optical interrogation method can be used to determine if particular chemical compounds affect a cell or population of cells. In this application, a cell or population of cells is exposed to at least one chemical compound. The cell and optical gradient are then moved relative to one another to determine whether the chemical compound affects the cell or population of cells. With respect to drug screening applications, the method can be used with a single type of cell population. This single population can be tested against a single chemical compound or multiple chemical compounds. Alternatively, a mixed population of different cell types may be tested with a single chemical compound or multiple chemical compounds. Quantitative analysis techniques can be used to determine which compounds show promising results.

The methods described herein are particularly useful in screening chemical compounds with relatively small cell populations. Testing can be performed by providing a series of sample cell populations. The series of sample cell populations are treated to the various chemical compounds. The treated cells are then subject to whole-cell cellular interrogation to determine whether the chemical compound affected the cell(s).

The preferred method of performing cellular interrogation is through optical interrogation which includes determining the optophoretic properties of the cell(s). The optophoretic properties of the cell(s) can be determined in any number of ways. In one preferred embodiment, the escape velocity of cell(s) is used to determine the optophoretic properties. The escape velocity (measured typically in μm/sec)is defined as the minimum speed at which an interrogated cell no longer tracks the moving optical gradient.

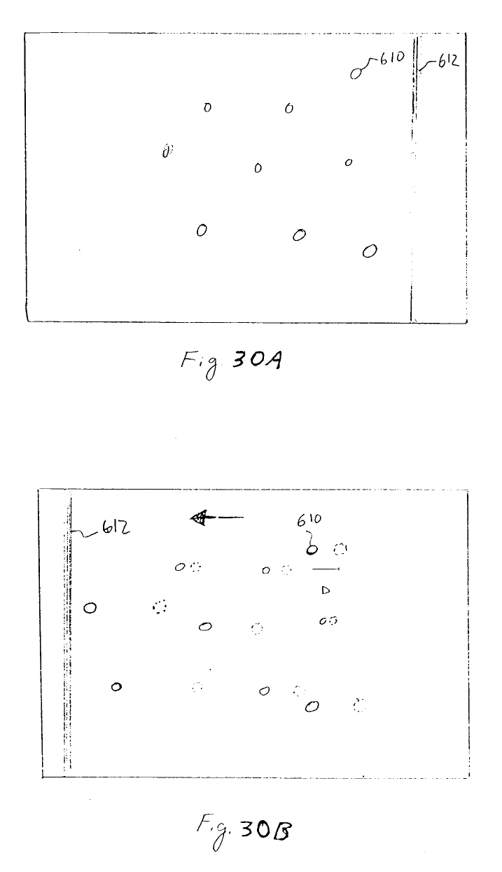

FIGS. 29A and 29B show optical interrogation of a group of cells 600 using a line scan. In this method, a moving optical gradient 602 (laser beam) in the form of a line is moved relative to the cells 600 to organize the cells 600 at position A. Preferably, the optical gradient 602 is moved at a speed which is sufficiently slow as to permit the capture and movement of the cells 600 to position A. Once the cells 600 are lined up at position A, the optical gradient 602 is moved in a stepwise fashion (in the direction of the arrow shown in FIG. 29B) in a pre-selected speed and distance to differentiate between cells or groups of cells having different escape velocities. FIG. 29B shows the end points of the steps at positions B, C, and D. FIG. 29B shows that three cells are effectively separated from the remaining cells as a result of the line scan procedure.

FIGS. 30A and 30B show optical interrogation of a group of cells 610 using a fast scan analysis. In this method, a moving optical gradient 612 (laser beam) in the form of a line is moved relative to the cells 610 Preferably, the cells 610 do not need to be lined up at position A prior to scanning. Instead, an image can be taken of the cells 610 prior to the scan to determine the starting position of each cell 610. Next the optical gradient 612 is rapidly moved in a continuous motion in the direction of the arrow shown in FIG. 30B. FIG. 30B shows the differential movement of the cells 612 (the initial or starting position of each cell 610 is shown in dashed lines). The distances traveled can be obtained by images taken of the cells 610 before and after the scanning process. The optical gradient 612 is moved at a speed that is higher than the escape velocity of all the cells 610 within the group. In this manner, all of the cells 610 are left behind the moving optical gradient 612. In an alternate fast scan embodiment, the cells 610 are initially lined up at a starting position A. After aligning the cells 610, the optical gradient 612 is rapidly moved and their distance of travel is measured by optical imaging. It should be understood that fast scan analysis may include multiple scans or “sweeps” of the cells 610. Generally, it has been observed that multiple scans produce larger cell movements.

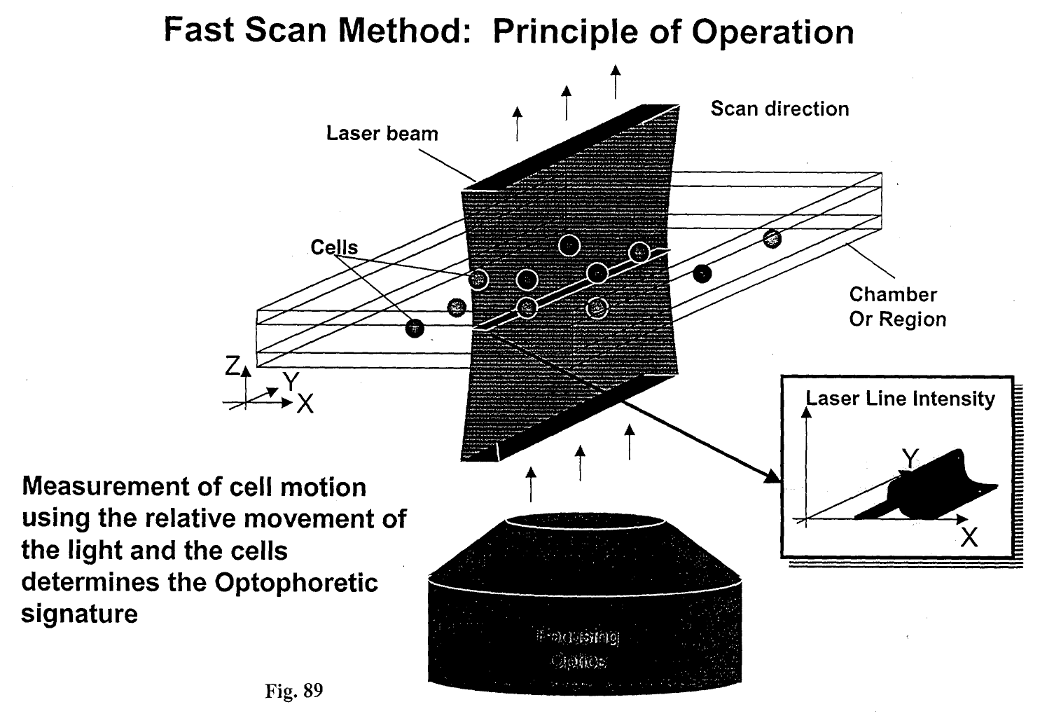

FIG. 89 shows the principles of operation of the fast scan method. In this method, cells are placed in a chamber or region 620 that serves as a sample holder. A beam of light such as, for example, a laser beam in the form of a line is projected into or onto the chamber or region using focusing optics 622. FIG. 89 also shows the laser line intensity in the x, y, and z axis directions. Relative movement is initiated either by moving the laser beam or, alternatively, by moving the chamber or region 620. Measurement of the displacement of each cell within the population provides a means of establishing an Optophoretic signature for each cell.

As stated above, the methods described herein allow for whole-cell interrogation of any number of cells including relatively small cell populations (preferably, less than about 1,000 cells). The methods described herein can be used on a variety of cell lines, including, for example, engineered cell lines, natural cell lines, and primary cells obtained from dissociated solid tissue.

Optical interrogation can also be performed on a panel of cells in order to determine whether a particular chemical compound or combination of compounds exhibits cellular toxicity. According to this method, a tissue panel of cells is provided. The tissue panel of cells is exposed to a chemical compound and then subject to whole-cell cellular interrogation. The interrogation determines whether the chemical compound exhibits cellular toxicity. In one preferred embodiment, the tissue panel of cells is comprised of cells from several target organs. Example target organs include the liver, kidney, heart, brain, and lungs.

The interrogation methods described herein are also useful in analyzing the time-dependent responses to chemical compounds for a population of cells. For example, a chemical compound that is a prospective drug candidate is exposed to a population of cells. The population of cells is optophoretically interrogated for a first time. The interrogation is repeated at a plurality of later times so as to establish a time-dependent response for the population of cells. This time-dependent response can also be coupled with varying concentrations of the chemical compound(s) to create a dose-dependent response as well.

With respect to drug screening applications of the method, a wide range of concentrations can be tested with the present method. Preferably, the range of concentration of the chemical compound(s) is within the range of about 1 femtomolar to about 100 micromolar.

Drug Discovery—Experiment 1 (Time Course Dependence of PMA Activation)

The objective of this experiment is to compare the escape velocities of U937 cells that have been treated with the phorbol ester, phorbol 12 myristate 13-acetate (PMA), to the escape velocity of untreated U937 cells. PMA activates the Protein Kinase C pathway and may cause the cells to go into rapid cell differentiation, which may be indicated by a shift in escape velocity. The effect of PMA activation was tested at three timepoints: no activation, 1 hour of activation, and 6 hours of activation. At the end of the activation period, the cells were centrifuged for 4 minutes at 10,000 rpm. The supernatant was then removed and the pellet resuspended in 1 ml PBS/1% BSA buffer. The cells were then centrifuged again and resuspended in buffer. The escape velocity and the cell size of cells from each tube were then calculated.

TABLE 1

| Time, in hours, of | Escape Velocity (μm/sec) | | |
|---|---|---|---|
| PMA activation | Average | SD | % CV |
| 0 | 18.3 | 2.9 | 16.0 |
| 1 | 15.3 | 0.7 | 4.7 |
| 6 | 28.0 | 1.5 | 5.3 |

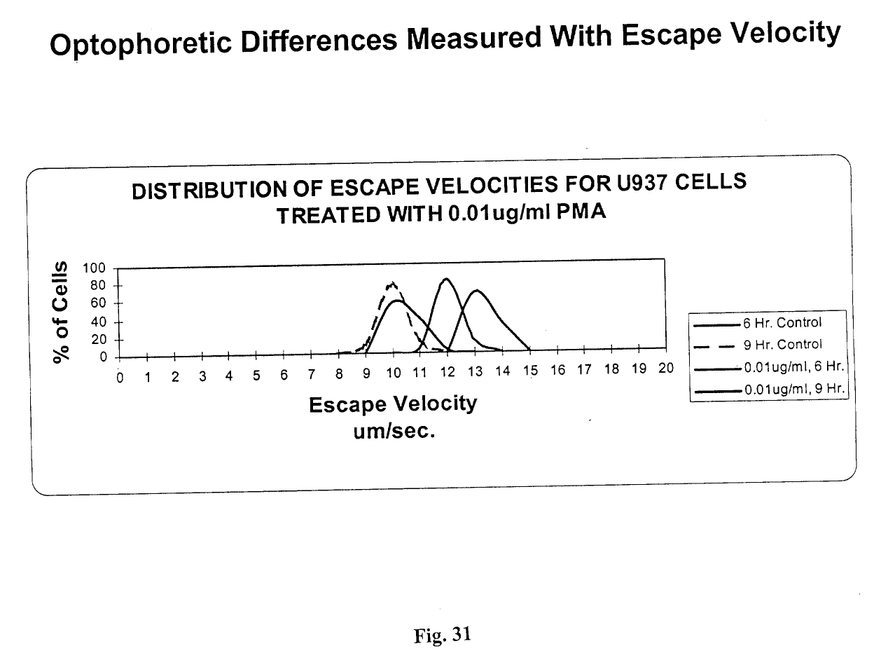

The time dependent effect of PMA was clearly present as can been seen in the shift of escape velocity to higher speeds over greater time periods. FIG. 31 shows the distribution of escape velocities for U937 cells treated with 0.01 μg/ml PMA at 6 and 9 hours-post treatment in addition to control cells (non-treated) at the same time intervals. The data show a clear trend toward higher escape velocities over time.

Drug Discovery—Experiment 2 (Time and Concentration Dependence of PMA)

This experiment was conducted to test the effect of PMA concentration on the escape velocity of U937 cells. PMA concentrations of 10 ng/ml, 1 ng/ml, 100 pg/ml, 10 pg/ml and 1 pg/ml were tested. The control sample received EtOH and not PMA.

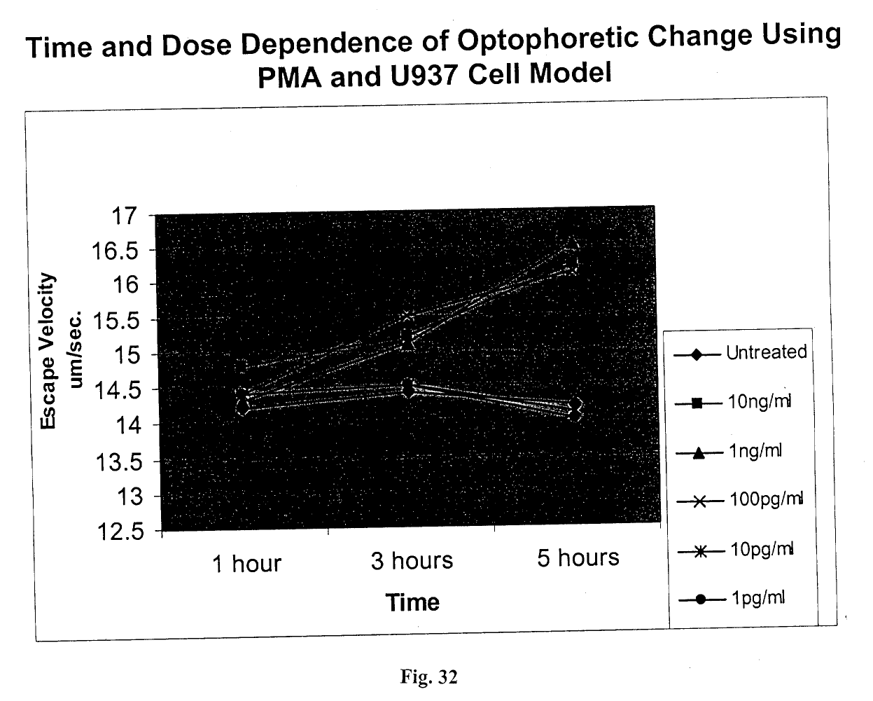

The PMA concentration was tested at three timepoints: 1 hour, 3 hours and 5 hours. At each timepoint, 300 μl of cells were removed from each flask. The cells were then spun for 5 minutes at 5000 rpm. The cells were then resuspended in 100 ml PBS/1% BSA and 25 ml of trypan blue. Table 2 below shows the escape velocities of the various concentrations at 1, 3, and 5 hours. FIG. 32 graphically shows the escape velocities for each time for the various concentrations (and control).

TABLE 2

| | 1 Hour | | | 3 Hour | | | 5 Hour | | |
|---|---|---|---|---|---|---|---|---|---|
| Conc. of | Activation | | | Activation | | | Activation | | |
| PMA | Ave. | SD | % CV | Ave. | SD | % CV | Ave. | SD | % CV |
| Control | 14.2 | 0.2 | 1.7 | 14.4 | 0.3 | 2.0 | 14.2 | 0.3 | 1.8 |
| 10 ng/ml | 14.8 | 0.4 | 2.5 | 15.2 | 0.3 | 2.3 | 16.2 | 0.4 | 2.3 |
| 1 ng/ml | 14.3 | 0.4 | 2.5 | 15.1 | 0.3 | 2.3 | 16.5 | 0.2 | 1.3 |
| 100 pg/ml | 14.4 | 0.4 | 2.5 | 15.5 | 0.4 | 2.5 | 16.1 | 0.3 | 1.7 |
| 10 pg/ml | 14.4 | 0.4 | 2.8 | 14.5 | 0.3 | 2.3 | 14.1 | 0.4 | 2.7 |

The concentration effect of the phorbol ester treatment on the cells can be seen three hours after treatment at the three highest concentrations tested: 10 ng/ml, 1 ng/ml and 100 pg/ml. The two lowest concentrations, 10 pg/ml and 1 pg/ml, which have concentrations below the physiological threshold for exhibiting a biological effect, showed no Optophoretic difference. Within those concentrations that did not have an effect, no difference was seen.

Drug Discovery—Experiment 3 (PMA Inhibition)

The objective of this experiment was to test the effect of bisindolymaleimide on cells exposed to PMA. Bisindolymaleimide is a Protein Kinase C inhibitor and should block the effect of PMA on U937 cells and their escape velocities. As shown in this and prior experiments, exposure of U937 cells to PMA results in an increase in the escape velocity of the cells. Therefore, it is anticipated that addition of bisindolymaleimide will reduce or eliminate the increase in escape velocity caused by PMA. This has in fact been observed in the data shown below.

Bisindolymaleimide was tested at two concentrations: 200 ng/ml and 50 ng/ml. Samples not treated with bisindolymaleimide received an equivalent amount of the carrier, MeOH, as a control. After the addition of bisindolymaleimide or MeOH, the samples were incubated at 37° C. 5% CO₂ for one hour before the addition of PMA, if any. The concentration of PMA tested was 10 ng/ml. Samples not receiving PMA received DMSO.

The following conditions were tested:

Control—No PMA and no bisindolymaleimide

PMA only

200 ng/ml bisindolymaleimide

50 ng/ml bisindolymaleimide

PMA+200 ng/ml bisindolymaleimide

PMA+50 ng/ml bisindolymaleimide

Once the flasks were prepared, they were incubated at 37° C. 5% CO₂ for four hours. At the four hour timepoint, 300 μl of cells were removed from each flask and pelleted at 5000 rpm for 5 minutes. The cells were then resuspended in 6 ml PBS/1% BSA and 60 μl trypan blue.

The escape velocities of the cells were as follows:

TABLE 3

| | Conc. of | | | |
|---|---|---|---|---|
| Conc. of PMA | Bisindolymalemide | Ave. | SD | % CV |
| 0 | 0 | 13.5 | 1.1 | 8.1 |
| 10 ng/ml | 0 | 15.4 | 0.9 | 5.7 |
| 0 | 200 ng/ml | 14.0 | 1.0 | 7.4 |
| 0 | 50 ng/ml | 13.9 | 0.6 | 4.3 |
| 10 ng/ml | 200 ng/ml | 14.5 | 0.9 | 6.0 |
| 10 ng/ml | 50 ng/ml | 15.1 | 1.0 | 6.9 |

The average escape velocity of the cells exposed to PMA only was 15.4 μm/sec. Flasks which contained PMA and bisindolymalimide showed a decreased escape velocity. The effect of bisindolymaleimide was also concentration dependent with a greater effect on the escape velocity shown with higher concentrations of bisindolymaleimide. The flask containing 200 ng/ml bisindolymaleimide and PMA had an average escape velocity of 14.5 μm/sec, and the flask containing 50 ng/ml bisindolymaleimide and PMA had an average escape velocity of 15.1 μm/sec.

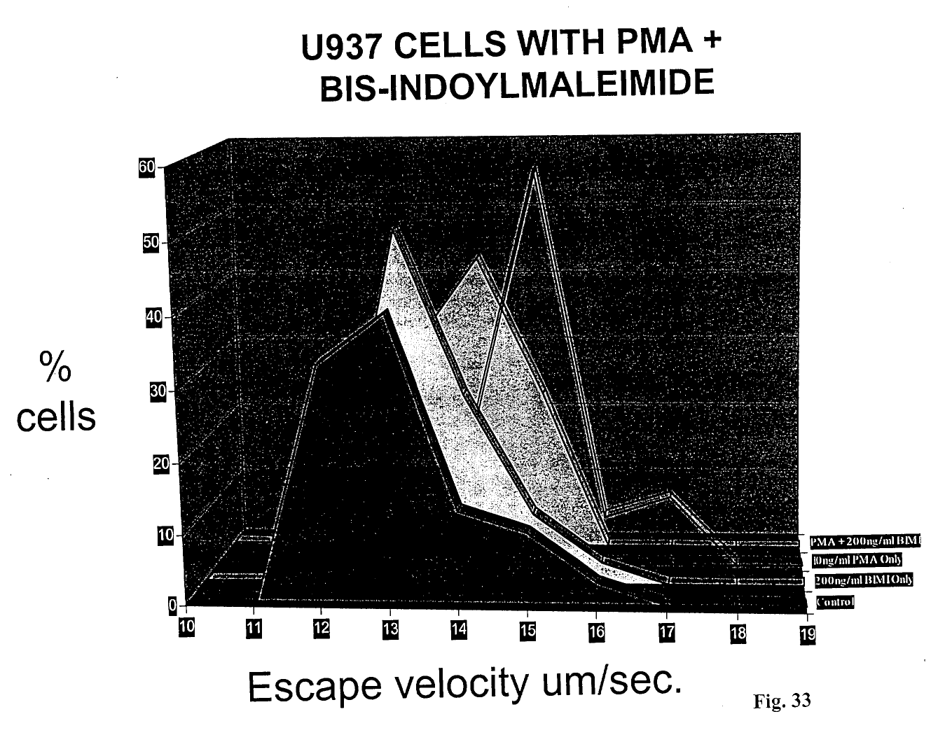

FIG. 33 shows the distribution of cells as a function of escape velocity for another experiment in which cells were treated with either 200 ng/ml of BIMI alone, 10 ng/ml of PMA alone, or 200 ng/ml of BIMI for 30 minutes followed by treatment with 10 ng/ml of PMA. The data show that pretreatment with BIMI blocked the optophoretic shift in escape velocity which treatment with PMA alone caused.

Drug Discovery—Experiment 4 (Effect of Camptothecin on U937 Cells)

The objective of this experiment is to test the effects of camptothecin on the escape velocities of U937 cells. Camptothecin inhibits DNA topoisomerase I and induces apoptosis. A control and three concentrations of camptothecin were tested: 4 mg/ml, 0.4 mg/ml and 0.04 mg/ml. After adding the camptothecin, the flasks are incubated at 37° C. 5% CO₂.

At the timepoints of 4 hours and 6 hours, 200 μl of cells are spun for 5 minutes at 5000 rpm. The cells are then resuspended in 75 ml PBS/1% BSA and 50 ml trypan blue.

The escape velocities were as follows:

TABLE 4

| | 4 Hour Activation | | | 6 Hour Activation | | |
|---|---|---|---|---|---|---|
| Conc. of | Avg. | | | Avg. | | |
| Camptothecin | (μm/sec) | SD | % CV | (μm/sec) | SD | % CV |
| Control | 11.52 | 0.45 | 3.95 | 11.79 | 0.52 | 4.40 |
| 0.04 μg/ml | 10.16 | 0.38 | 3.76 | 9.52 | 0.45 | 4.78 |

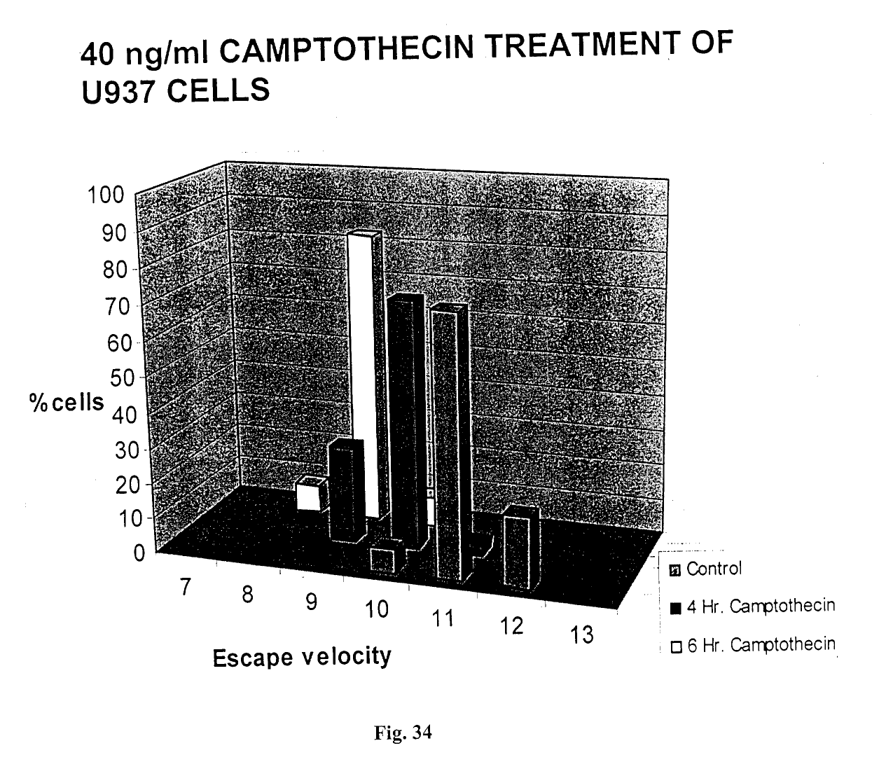

The two controls show little variance in escape velocity. The treated cells demonstrate a shift to lower escape velocities over time. FIG. 34 shows the distribution of U937 cells that were treated with 40 ng/ml of camptothecin at 4 and 6 hours as compared to a control. Again, a shift to lower escape velocities over time is seen with the camptothecin-treated cells.

Drug Discovery—Experiment 5 (TNF-α Effect on Jurkat Cells)

After 48 hours of incubation, the cells were removed and centrifuged for 5 minutes at 5,000 rpm. Then the cells were resuspended in PBS/1% BSA and trypan blue. A higher power setting, 140 mW instead of 100 mW, was used for this experiment. The escape velocities were measured as follows:

TABLE 5

| | Ave. (Escape | | |
|---|---|---|---|
| | Velocity) | SD | % CV |
| Control | 9.8 | 0.4 | 4.6 |
| 500 ng/ml TNF | 11.2 | 0.6 | 5.7 |
| 250 ng/ml TNF | 11.4 | 0.4 | 3.2 |
| 100 ng/ml TNF | 10.2 | 0.4 | 4.1 |

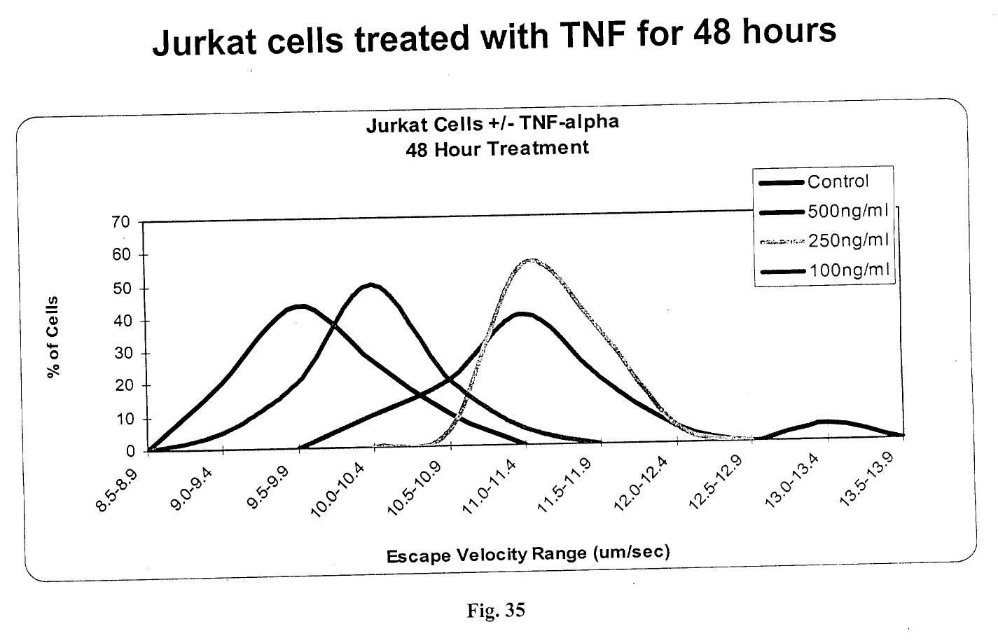

FIG. 35 shows the distribution of cells in various escape velocity ranges for the control, 500 ng/ml TNF, 250 ng/ml TNF, and 100 ng/ml TNF Jurkat treated cells at 48 hours.

A concentration effect was shown in that increased concentrations of TNF-alpha showed increased escape velocity at 48 hours incubation.

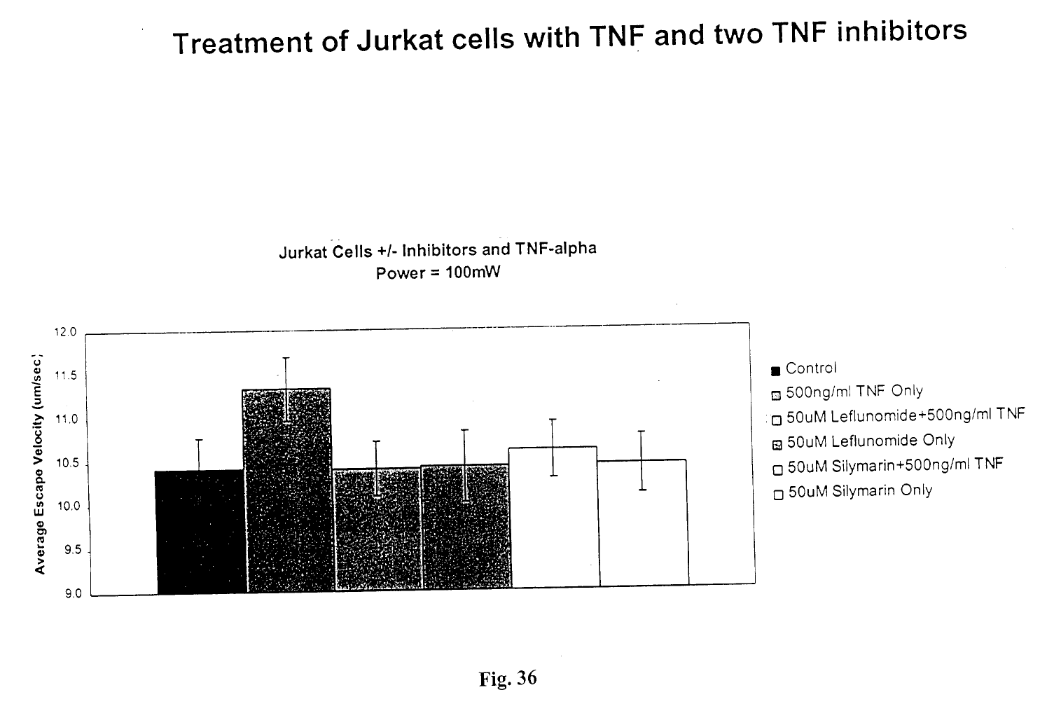

FIG. 36 shows the effect of two TNF inhibitors, Leflunomide and Silymarin used in conjunction with TNF. The previous experiments (Table 5) demonstrate that TNF generally increases the escape velocity of cells. In this experiment Leflunomide and Silymarin were added with TNF to see if the anticipated increase in escape velocity measurements could be counteracted by the presence of the TNF inhibitors. The data shown in FIG. 36 confirm that Leflunomide and Silymarin mitigate the increase in escape velocity caused by TNF.

Drug Discovery—Experiment 6 (Effect of Sodium Salicylate on U937 Cells)

This experiment is designed to compare the effects of two concentrations of sodium salicylate on U937 cells. Sodium salicylate was prepared at two concentrations: 20 mM and 5 mM. The escape velocities of the cells was tested at 5 hours, 24 hours and 47 hours. At each timepoint, 30 μl of sample was pelleted at 5,000 rpm for 5 minutes and then resuspended in PBS/1% BSA and trypan blue.

The escape velocities were as follows:

TABLE 6

| Conc. of | | | | | | | | | |
|---|---|---|---|---|---|---|---|---|---|
| Sodium | 5 hours | | | 24 hours | | | 47 hours | | |
| Salicylate | Ave. | SD | % CV | Ave. | SD | % CV | Ave. | SD | % CV |
| Control | 14.3 | 0.8 | 5.7 | 14.1 | 1.0 | 7.0 | 14.4 | 0.7 | 4.7 |
| 20 Mm | 13.3 | 0.8 | 6.0 | 16.0 | 1.2 | 7.6 | | | |
| 5 mM | 14.1 | 0.6 | 4.1 | 15.2 | 0.8 | 5.2 | 13.7 | 0.8 | 5.5 |

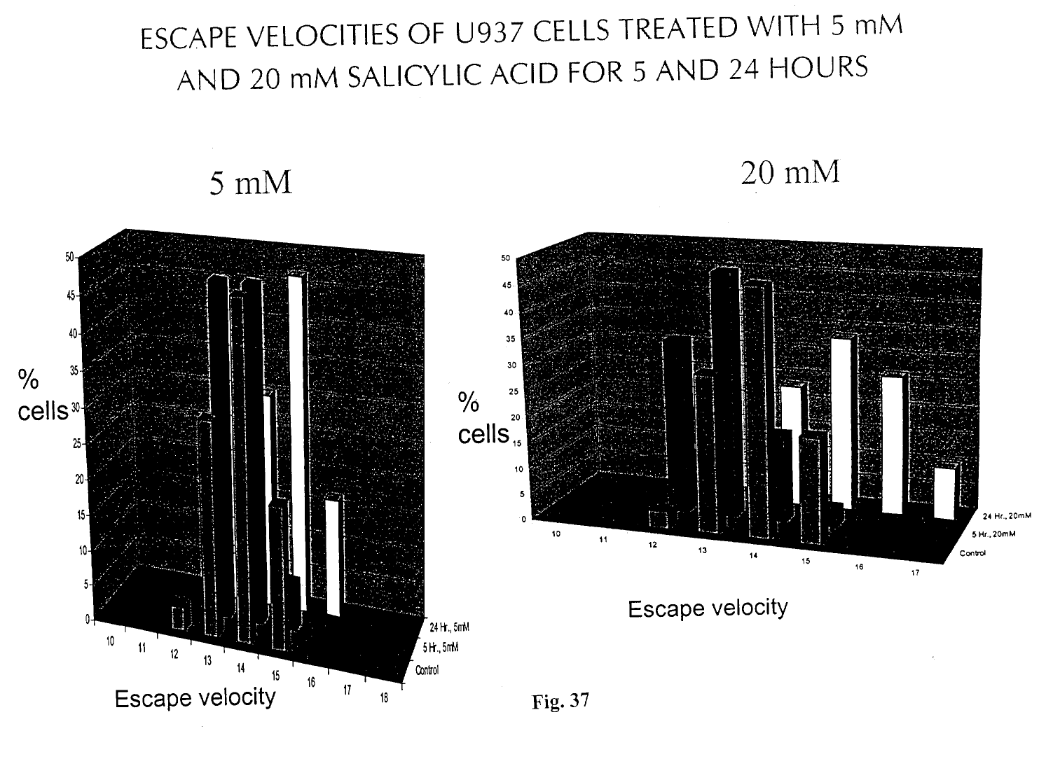

No data was collected for 20 mM at 47 hours because all cells were dead and stained with trypan blue. FIG. 37 shows the distribution of escape velocities of U937 cells treated with 5 mM and 20 mM salicylic acid for 5 and 24 hours. Salicylic acid is a chemical compound that has a mild effect on cells. The results indicate that Optophoresis is able to detect a slight but statistically significant shift in escape velocities in response to the presence of salicylic acid. This suggests that Optophoresis is sensitive to small biological changes caused by chemical compounds that affect cells in a relatively minor way.

Drug Discovery—Experiment 7 (Time and Concentration Dependence of Paciltaxel on K562 Cells)

In this experiment, the effect of time and concentration of paciltaxel was tested using K562 cells. Paciltaxel is a chemotherapeutic agent whose mechanism of action is the inhibition of tubulin polymerization and the subsequent disruption of the cytoskeleton in cells. The timepoints tested were 4 hours, 23 hours, 30 hours and 47 hours. The concentrations of paciltaxel used in the experiment were 10 nM, 1 nM, 100 pM, and 10 pM. At each timepoint, 300 μl of cells were removed from each sample and centrifuged for 5 minutes at 5,000 rpm. The cells were then resuspended in PBS/1% BSA and trypan blue.

The following escape velocity values were collected:

TABLE 7

| Conc. Of | 4 Hours | | | 23 Hours | | | 30 Hours | | | 47 Hours | | |
|---|---|---|---|---|---|---|---|---|---|---|---|---|
| paciltaxel | Ave | SD | % CV | Ave | SD | % CV | Ave | SD | % CV | Ave | SD | % CV |
| Control | 15.6 | 0.6 | 3.6 | 15.9 | 0.3 | 1.9 | 15.9 | 0.3 | 1.8 | 15.9 | 0.3 | 1.8 |
| 10 nM | 15.3 | 0.5 | 3.1 | 16.9 | 0.3 | 2.0 | 17.4 | 0.4 | 2.5 | 17.1 | 0.3 | 2.0 |
| 1 nM | 15.6 | 0.4 | 2.9 | 16.9 | 0.4 | 2.2 | 16.9 | 0.3 | 1.5 | 16.7 | 0.4 | 2.3 |
| 100 pM | 15.9 | 0.4 | 2.6 | 17.1 | 0.4 | 2.3 | 17.6 | 0.4 | 2.4 | 17.0 | 0.3 | 1.6 |
| 10 pM | 15.7 | 0.4 | 2.6 | 15.7 | 0.3 | 2.1 | 16.0 | 0.3 | 1.6 | 16.2 | 0.7 | 4.1 |

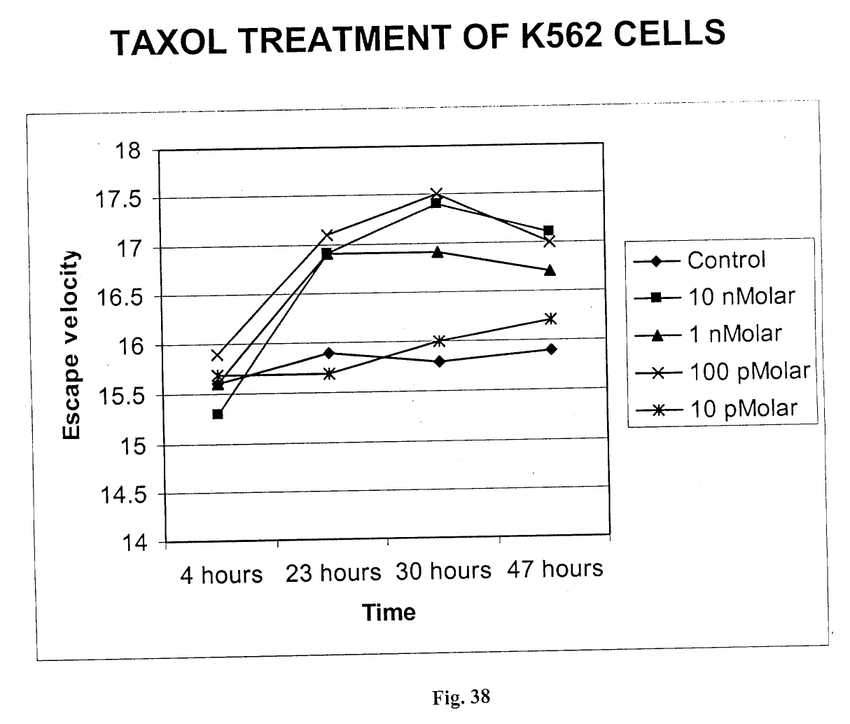

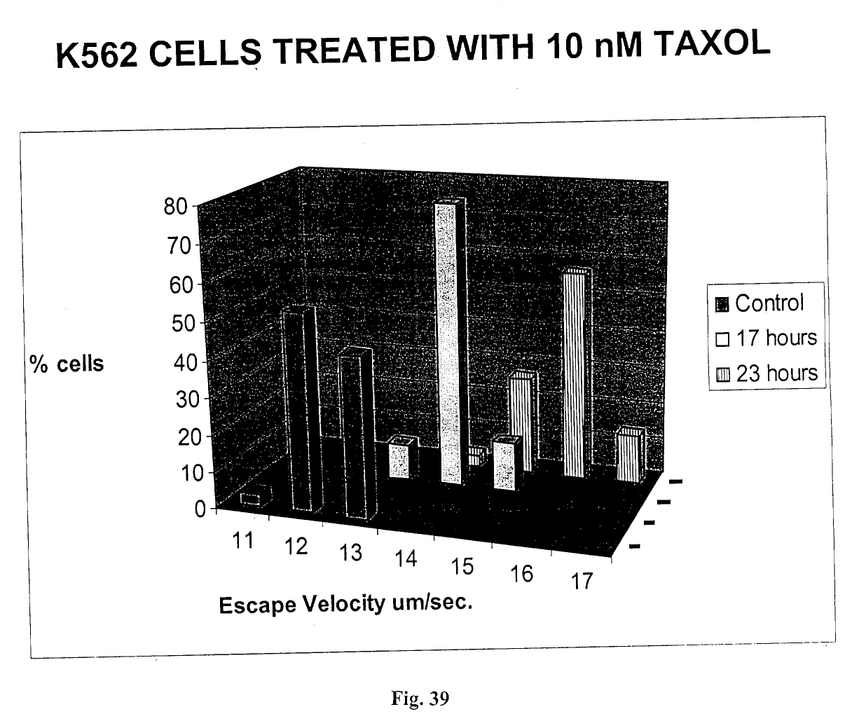

FIG. 38 shows the time course variation in escape velocity for varying concentrations of paciltaxel. FIG. 39 shows the distribution of cells vs. escape velocity for K562 cells that were treated with 10 nM of paciltaxel at 17 and 23 hours. Escape velocity tends to increase as time progresses. The data show both a time and dose dependent optophoretic effect of paciltaxel on cells.

Drug Discovery—Experiment 8 (Effect of Gleevec (imatinib mesylate) on K562 , BV-173, EM-3, and U-937 Cells—Escape Velocity)

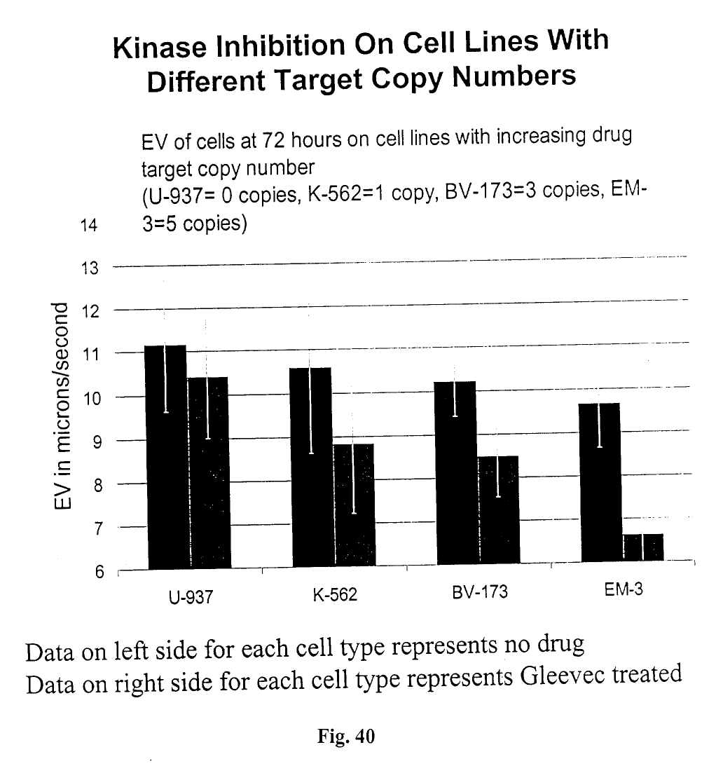

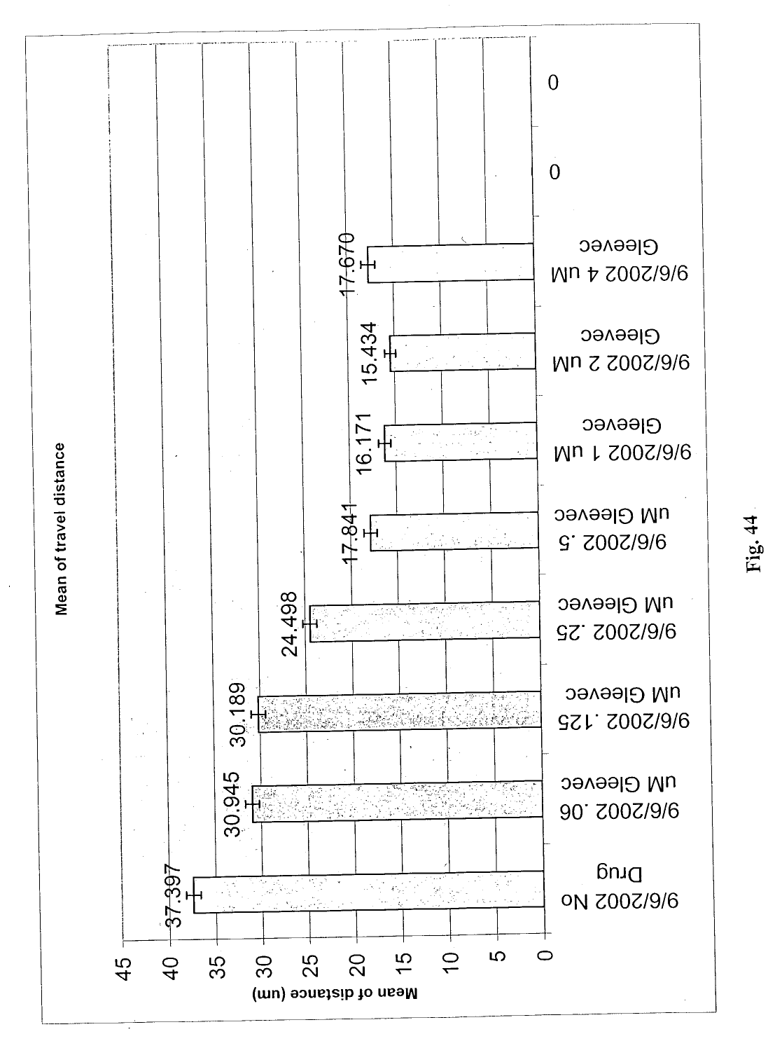

This experiment was designed to test the effect of imatinib mesylate (known commercially as Gleevec) on K562 , BV-173, EM-3, and U-937 cells. Each cell type has different copy numbers of the gene that produces Bcr-Abl tyrosine kinase enzyme. The U-937 cells have no copies of the gene. The K-562 line of cells has, on average, one copy of the gene per cell. The BV-173line of cells has, on average, three copies of the gene per cell. The EM-3 cell line has, on average, five copies of the gene per cell. Gleevec acts to inhibit cellular growth through its inhibition of the Bcr-Abl tyrosine kinase enzyme. The effect of Gleevec was tested by measuring the escape velocity of the control and treated cells 72 hours after exposure to Gleevec. FIG. 44 illustrates the measured escape velocities (average) for each of the four cell types. As seen in FIG. 40, the Gleevec-treated cells show a significantly lower escape velocity as compared to non-treated cells. This effect was seen in all four cell types. In addition, the decrease in escape velocity for the Gleevec-treated cells was more pronounced in those cell lines that had higher copy numbers of the Bcr-Abl gene.

Drug Discovery—Experiment No. 8.1 (Effect of Gleevec (imatinib mesylate) on K-562, BV-173, EM-3, and U-937 Cells-Fast Scan)

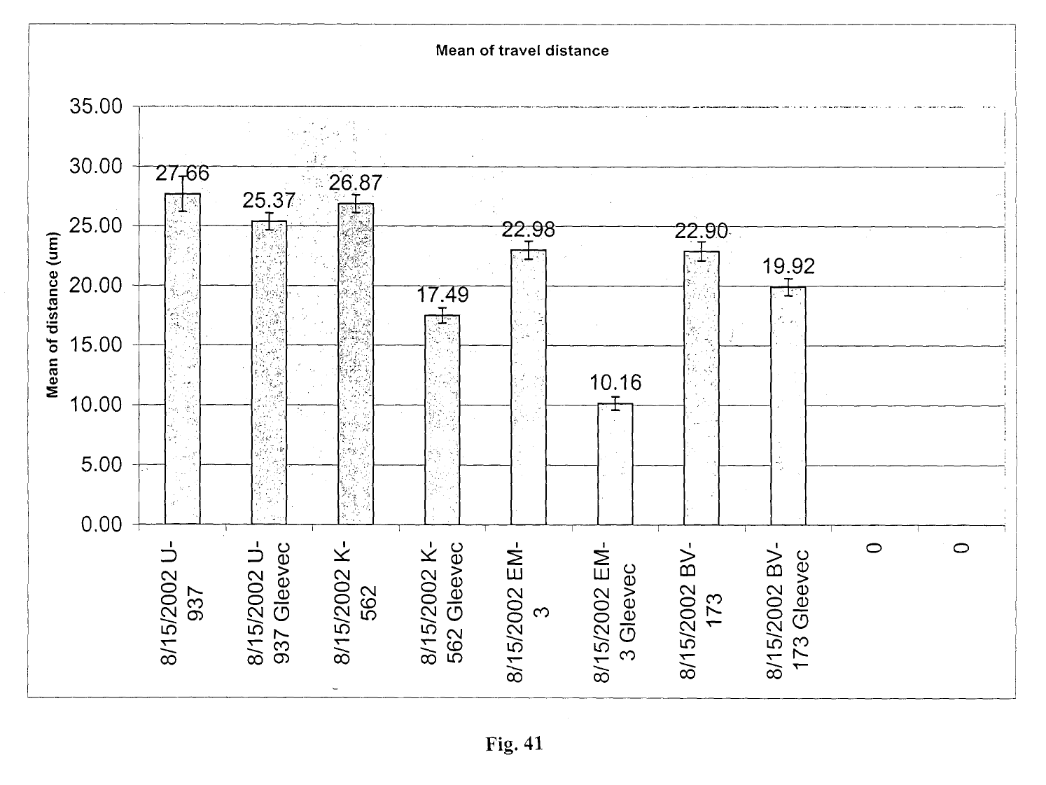

This experiment was conducted using fast scan analysis after 48 hours of treatment of the K-562, BV-173, EM-3, and U-937 cell lines with 1 μM Gleevec. FIG. 41 illustrates the measured (mean) travel distances for the control groups as well as the Gleevec-treated cells. As seen in FIG. 41, with the exception of the U-937 cells (which do not contain any copies of the gene that produces Bcr-Abl tyrosine kinase enzyme), the Gleevec-treated cells have lower mean travel distances as compared to their non-treated controls. This data confirms that an inhibitor of the Bcr-Abl tyrosine kinase enzyme, Gleevec, induced Optophoretically measurable changes (as measured using fast scan analysis) in the Bcr-Abl positive cell lines after 48 hours of treatment.

Drug Discovery—Experiment 9 (Effect of Gleevec and Other Kinase Inhibitors on EM-3 Cells—Fast Scan)

In this experiment, a fast scan analysis was performed to measure the differential effect of Gleevec on the EM-3 cell line. A first group of cells were treated with 1 μM Gleevec. A second group of cells were treated with 1 μM Src-Family Protein Tyrosine Kinase Inhibitor Set, obtained from CALBIOCHEM (Cat. No. 567816). The Src Set contained four inhibitors, namely, Genistein, Herbimycin A, Streptomyces sp., PP2, and PP3. A third group of cells were treated with 1 μM Staurosporine. A fourth group of cells were treated with 1 μM TK Inhibitor Set, also obtained from CALBIOCHEM (Cat. No. 657021). The TK Inhibitor Set contained five inhibitors, Genistein, PP2, AG490, AG1296, and AG1478. The TK Inhibitor Set contains a general tyrosine kinase inhibitor and a series of inhibitors which are selective for various tyrosine kinases that are important in cellular signaling. Measurements were taken after 48 hours of exposure. Table 8 shown below illustrates the mean travel distances for each cell group after 48 hours of exposure.

TABLE 8

| | | | 1 μM Src | | |
|---|---|---|---|---|---|
| | No Drug | 1 μM Gleevec | Set | 1 μM Staurosporine | 1 μM TK Set |
| No. of | 314 | 308 | 340 | 347 | 316 |
| Cells | | | | | |
| Mean | 27.014 | 13.199 | 35.446 | 9.250 | 26.556 |
| Distance | | | | | |
| (μm) | | | | | |
| Std. Dev. | 9.741 | 10.480 | 15.264 | 6.427 | 10.943 |
| CV % | 36.1 | 79.4 | 43.1 | 69.5 | 41.2 |

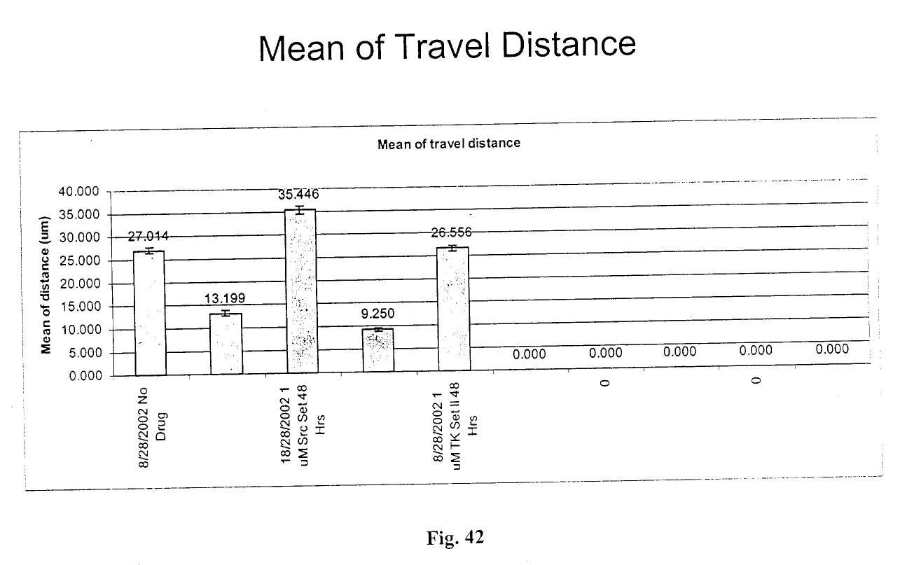

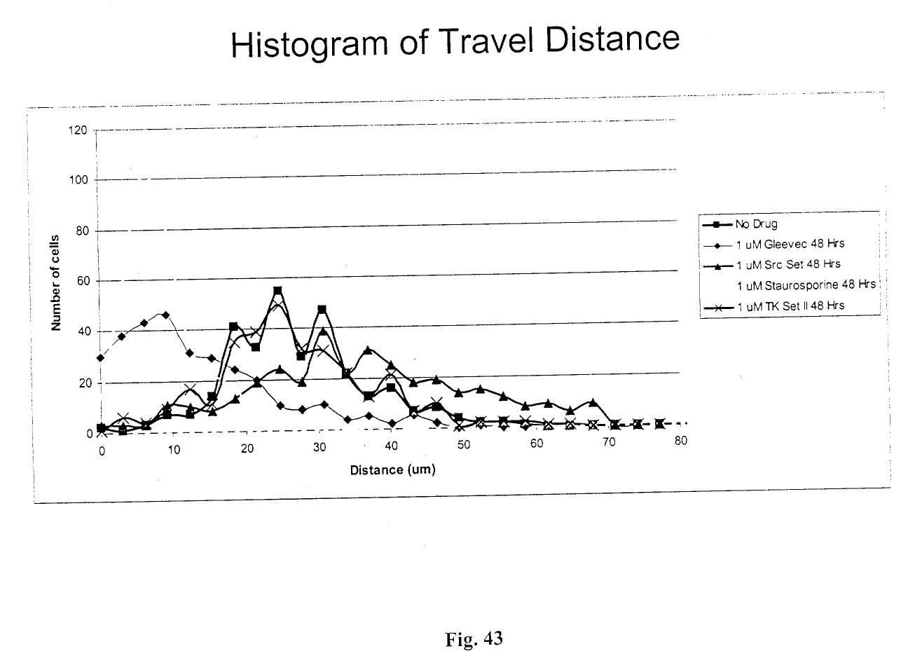

FIG. 42 shows the mean travel distance for the four treated groups of cells as well as the control. FIG. 43 shows a histogram of the travel distance for each of the various cell groups. The data show approximately the same degree of shift in mean distance traveled for the Gleevec treated cells as for the Staurosporine treated cells. Staurosporine is a very broad range inhibitor of kinases, and Gleevec's target is a known kinase. In contrast, the two other kinase inhibitors, Src Set and TK Set, which are highly selective sets of kinase inhibitor drugs, do not cause a decrease in the mean distance traveled. Thus, optophoretic interrogation demonstrates that Gleevec's effect is specific to this cell line (EM-3).

Drug Discovery—Experiment No. 9.1 (Dose Dependent Response of Gleevec (imatinib mesylate) on EM-3 Cells—Fast Scan)

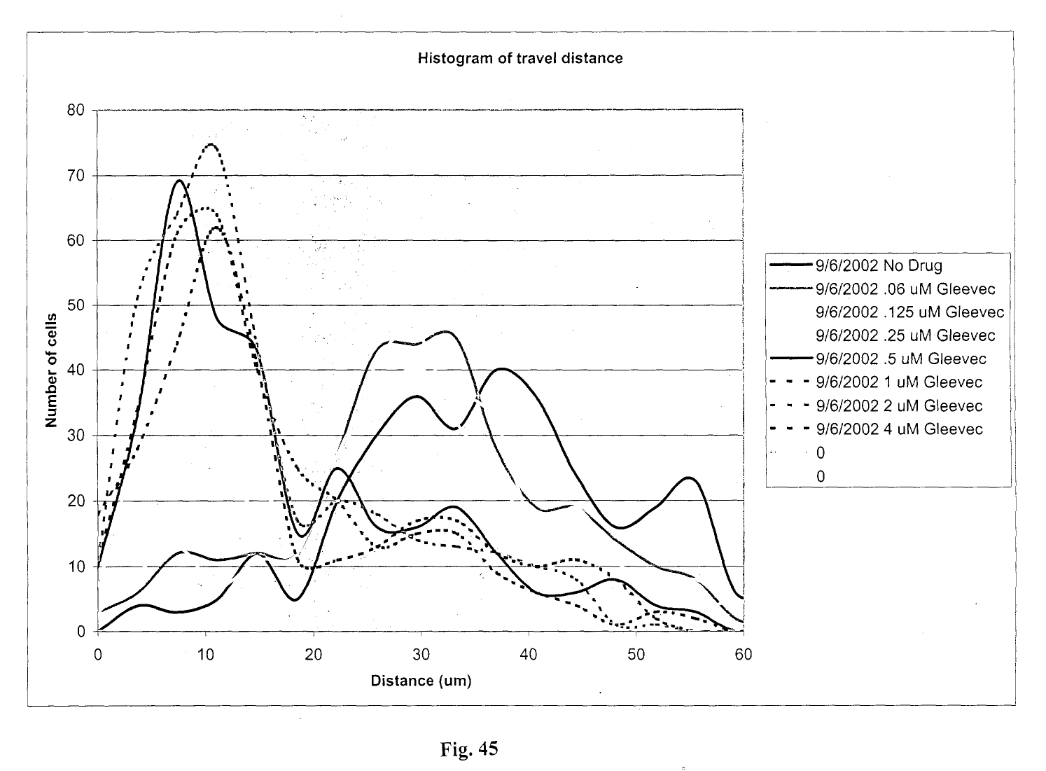

In this experiment, various concentrations of Gleevec were administered to cells from the EM-3 cell line. The concentrations of Gleevec tested included 0.06 μM, 0.125 μM, 0.25 μM, 0.5 μM, 1 μM, 2 μM, and 4 μM. The control sample comprised EM-3 cells that were not treated with Gleevec. Fast scan analysis was performed after 48 hours of incubation. FIG. 44 illustrates the mean travel distances for the treated and untreated EM-3 cells. The data show a dose-dependent response to varying Gleevec concentration as measured by fast scan analysis. Those cell lines treated with higher concentrations of Gleevec showed generally larger decreases in mean travel distances. It also appears that further decreases in mean travel distances was not seen once the concentration of Gleevec reached a certain level (about 1 μM). FIG. 45 shows a histogram of the travel distances for the various tested concentrations of Gleevec.

Drug Discovery—Experiment 10 (Toxicity of Liver Cells Upon Exposure to Ketoconazole)

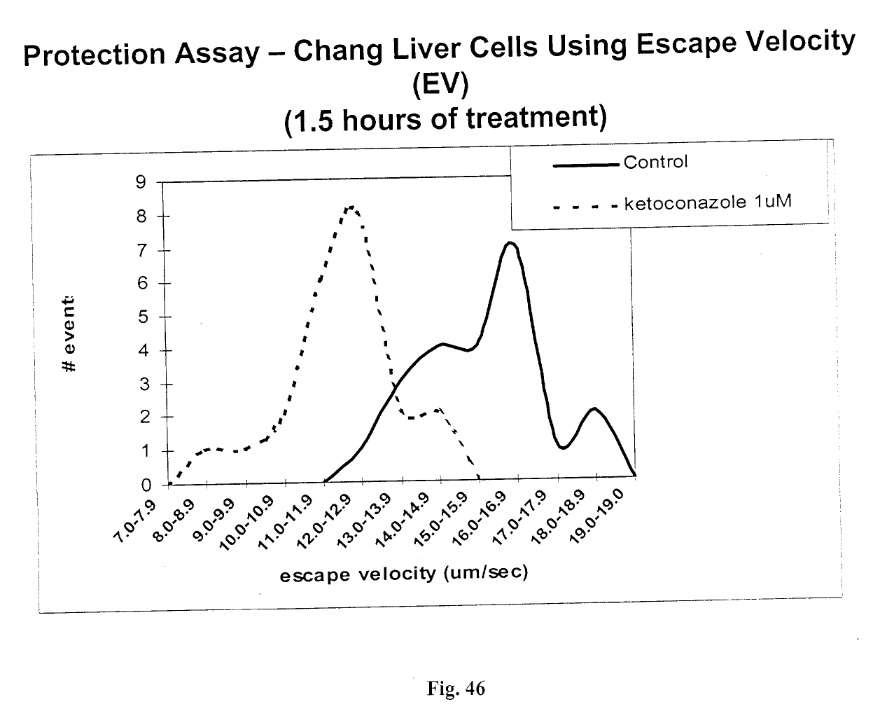

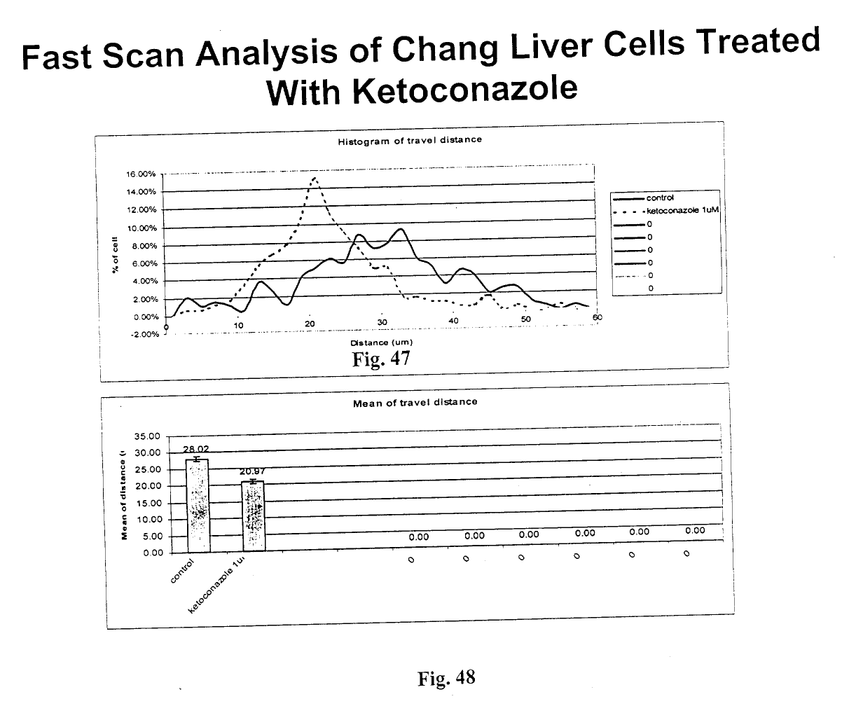

This experiment is designed to test the effect of ketoconazole on human liver cells. Ketoconazole induces toxicity in liver cells. The sample cells were Chang liver cells. The concentration of ketoconazole used to treat the cells was 1 μm. Escape velocity and fast scan protocols were applied to determine the impact, if any, of ketoconazole treatment. FIG. 46 shows the distribution of cells as a function of escape velocity for both the control and the Chang liver cells treated with 1 μm of ketoconazole after an 1.5 hours of treatment. The ketoconazole-treated cells showed a marked decrease in escape velocity. Consequently, Optophoresis was able to detect an effect on the escape velocity of treated cells within 1.5 hours of exposure to ketoconazole. FIGS. 44-45 show the fast scan results of ketoconazole-treated Chang liver cells. Specifically, FIG. 47 shows the distribution of cells as function of travel distance. The ketoconazole-treated cells generally traveled a smaller distance as compared to the non-treated control. FIG. 48 shows the mean travel distances for the control (28.02 μm) and the ketoconazole-treated cells (20.97 μm).

Drug Discovery—Experiment 11 (Dose Response Curve of Topotecan)

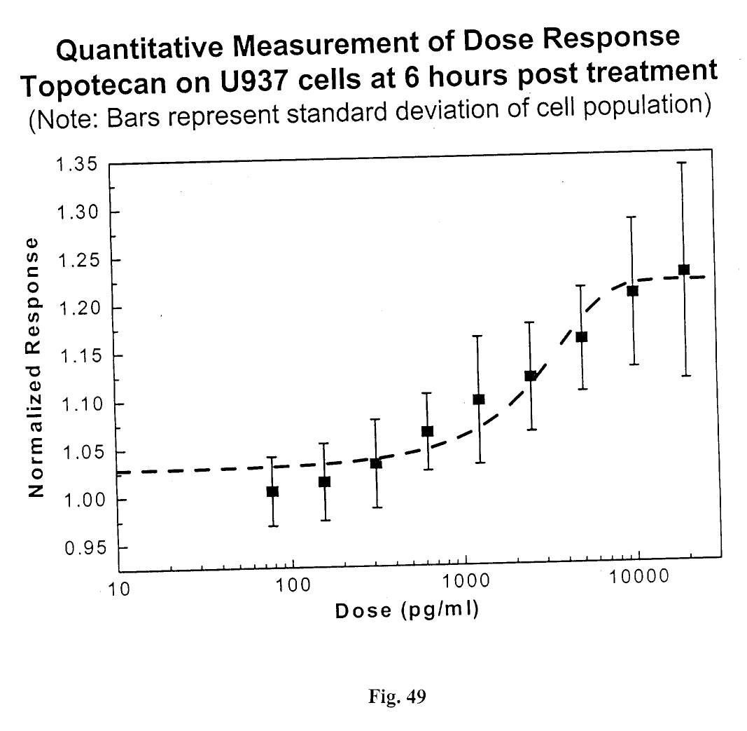

The purpose of this experiment was to see if optical interrogation could be used to develop a dose response curve for a particular chemical compound. In this experiment, U937 cells were treated with varying concentrations of the drug topotecan. After 6 hours of exposure, escape velocity measurements were taken. FIG. 49 shows a dose response curve that was generated from this data. In this graph, the dose was plotted against the normalized response (1.00 represents the control). As can be seen in FIG. 49, a typical s-shaped dose response curve was generated.

Drug Discovery—Experiment 12 (Dose Response Curve of PMA)

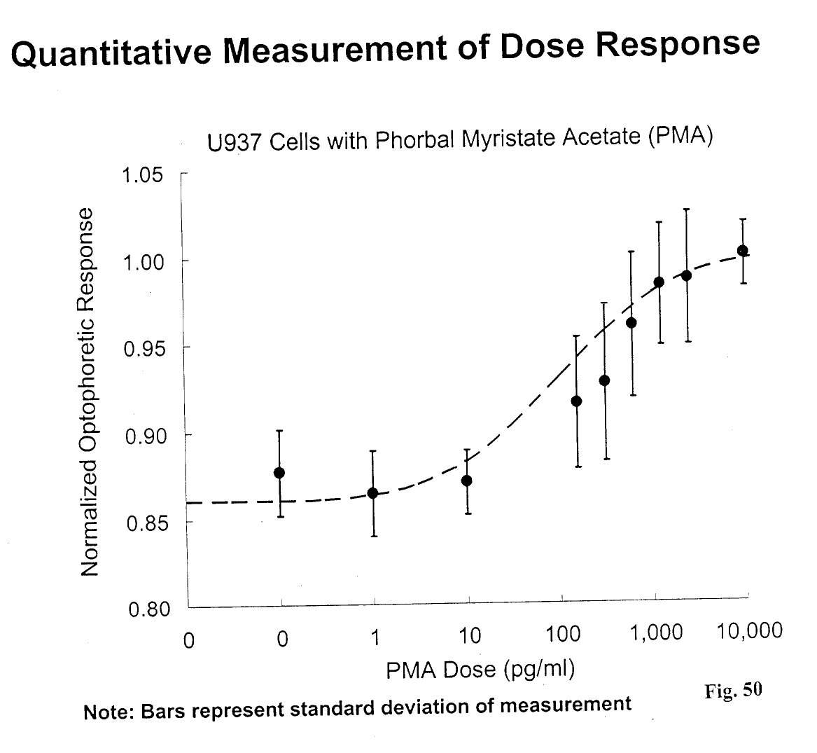

FIG. 50 shows the dose response curve for U937 cells treated with varying concentrations of phorbol myristate acetate (PMA). Escape velocity measurements were taken of the cells after exposure to PMA. The normalized Optophoretic response was plotted against the PMA dose response. As can be seen in FIG. 50, a typical s-shaped does response curve was generated.

Drug Discovery—Experiment 13 (Quantitative Determination of PKC Activation by Optophoresis)

Optophoretic analysis was used to develop a full dose response curve for U937 cells treated with PMA. U937 cells were incubated with varying amounts of PMA dissolved in DMSO. The following concentrations of PMA were tested: 0.156 ng/ml, 0.312 ng/ml, 0.625 ng/ml, 1.25 ng/ml, 2.5 ng/ml, 5 ng/ml, and no PMA (control). The cells were incubated with the PMA solutions (or simply DMSO in the case of the control) for five hours. At the end of the incubation period, the cells were centrifuged down for five minutes at 5000 rpm. The supernatent was then removed and the pellet resuspended in 1 ml PBS/1% BSA buffer plus 1:10 trypan blue. The cells were then loaded onto a cover slip containing agarose that was spin-coated onto the surface thereof. Escape velocity measurements were then taken using a 100 mW laser having a beam diameter of 28 pixels. Table 9 reproduced below sets forth the measured escape velocities (μm/sec) for each of the various PMA concentrations.

TABLE 9

| Cell | | .156 | .312 | .625 | 1.25 | 2.5 | 5 |
|---|---|---|---|---|---|---|---|
| Number | No PMA | ng/ml | ng/ml | ng/ml | ng/ml | ng/ml | ng/ml |
| Average | 15.8 | 15.8 | 16.0 | 16.6 | 17.0 | 17.0 | 17.3 |
| SD | 0.8 | 0.7 | 0.8 | 0.7 | 0.6 | 0.7 | 0.5 |
| % CV | 5.0 | 4.1 | 4.9 | 4.3 | 3.5 | 3.8 | 2.9 |

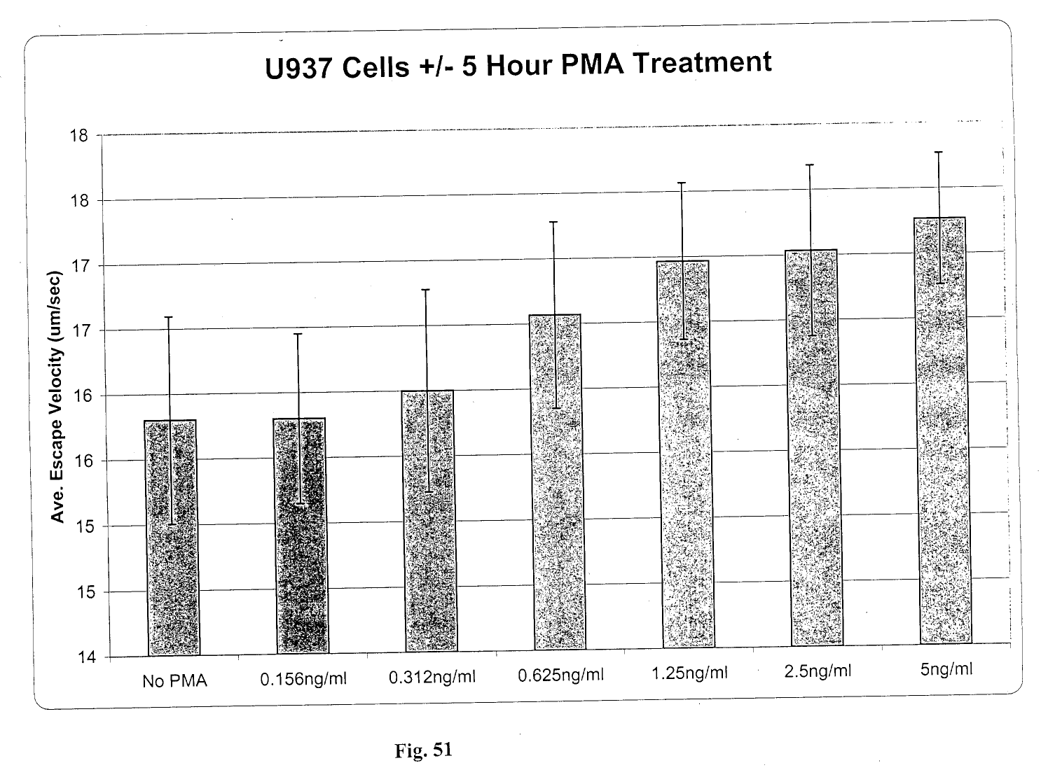

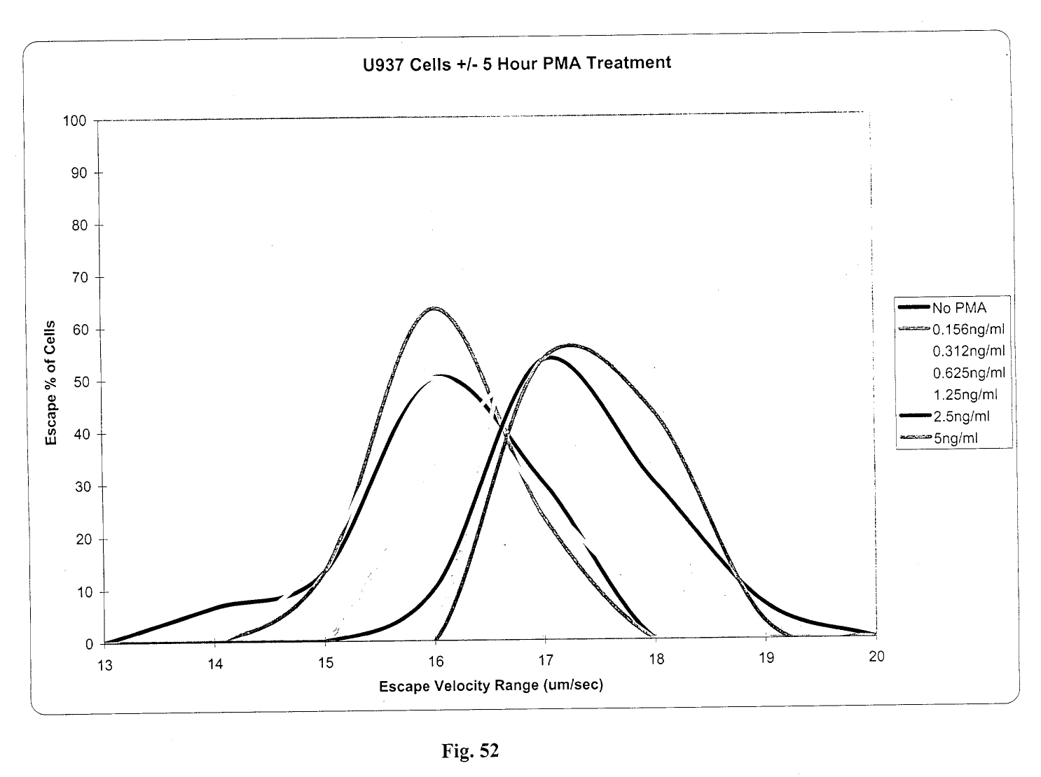

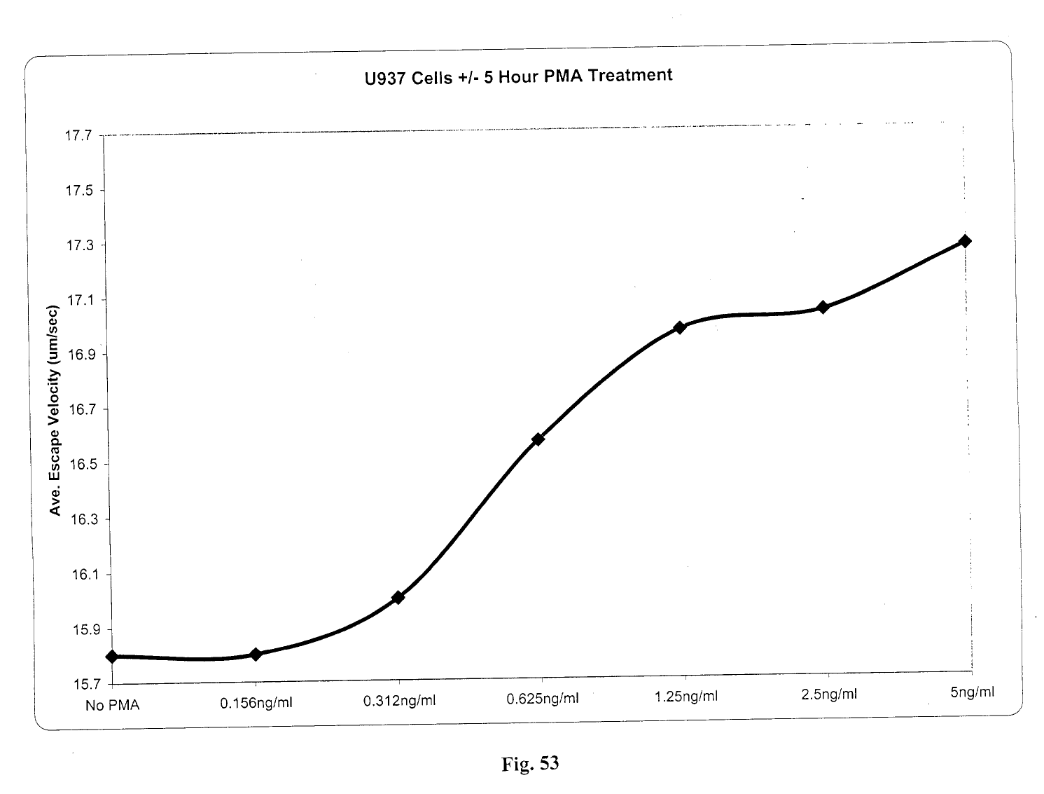

FIG. 51 graphically illustrates the average measured escape velocities for the various PMA concentrations (including the control). Error bars indicate the 95% confidence level. FIG. 52 illustrates the distribution of cells as measured by cell percentage as a function of measured escape velocity range for each of the PMA concentrations and the control. The data show a consistent trend toward higher escape velocities for larger concentrations of PMA. A significant shift in escape velocity is seen at PMA concentrations exceeding 0.625 ng/ml. FIG. 53 illustrates a graph of the measured escape velocity as a function of PMA concentration. FIG. 53 shows that escape velocity varies in response to PKC activation in a dynamic, dose dependent manner.

The method described above may be used to quantitatively determine the level of PKC activation in cells in response to exposure to PKC activating compound using a moving optical gradient. The method includes the steps of first providing a series of cell samples and exposing the series of cell samples to different concentrations of the PKC activating compound. After a period of incubation from about one to several hours, the cells and the optical gradient are moved relative to each other so as to cause displacement of at least some of the cells. Next, the displacement of at least a portion of the displaced cells is measured for each of the different concentrations. This data is used to generate a dose response curve of the measured displacement as a function of the concentration of the PKC activating compound. From the dose response curve, one is able to calculate one or more numerical values that correlate to PKC potency and/or efficacy. For example, the dose response curve can be used to calculate the EC₅₀, Hill slope, or plateau value relative to a standard compound.

The EC₅₀ value of the PMA dose response curve shown in FIG. 53 is about 0.625 ng/ml. This value is consistent with values obtained using other non-Optophoretic methods. While the full dose response curve shown in FIG. 53 is for PMA, the same techniques described above can be employed with other PKC activating compounds. In this regard, Optophoretic analysis is able to score and rank other PKC activators, whether known or unknown. In addition, while the dose response curve shown in FIG. 53 measures escape velocity as a function of concentration, the same principles may be applied to other optical interrogation techniques such as fast scan analysis. If fast scan analysis is used, the dose response curve would generally comprise the mean travel distance plotted as a function of concentration.

Drug Discovery—Experiment 14 (Optophoretic Characterization of Topoisomerase Inhibitors)

Inhibition of topoisomerase is a known mechanism of cancer therapy. Current in vitro cell-based assays measure cell death, metabolic viability, DNA strand breaks or membrane integrity, using various dyes, intercalators and external reagents. These assays are very time and labor intensive and require a significant amount of sample preparation as well as utilization of reagents. For example, DNA strand break assays require laborious subsequent analysis via gel electrophoresis. Moreover, these assays are destructive to the cells, thereby preventing additional analysis to establish, for example, their tumorgenicity (e.g., clonal outgrowth). Optophoretic analysis techniques, on the other hand, can provide a way of identifying chemical compounds that inhibit topoisomerase activity without the need for potentially damaging labels or time and labor intensive processing. These Optophoretic techniques can also be used to identify cells or cell lines that are resistant to known topoisomerase inhibitors.

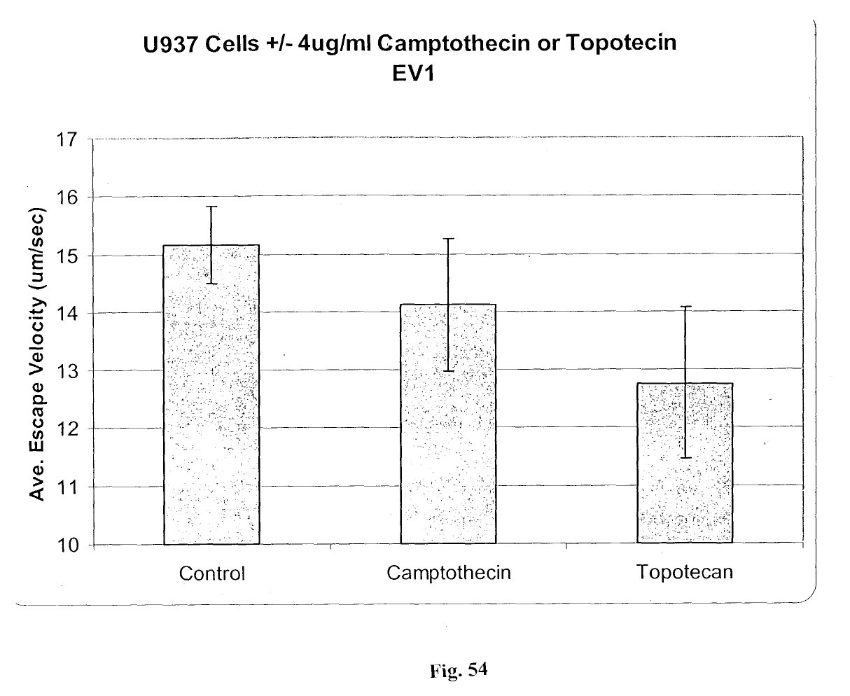

In a first experiment, a first group of U937 cells were incubated in modified RPM1-1640 media containing 10% fetal bovine serum (media) with 4 μg/ml of camptothecin first dissolved in DMSO then diluted to 0.1% (v/v) final DMSO. Camptothecin is a known inhibitor of topoisomerase. Another group of U937 cells were incubated with 4 μg/ml of topotecan, a chemical analog of camptothecin that has known higher potency as compared to camptothecin. Both groups of cells were incubated with compound in 0.1% DMSO (or simply 0.1% DMSO in the case of the control) for four hours. At the end of the incubation period, the cells were centrifuged down for 3-5 minutes at 5000 rpm in a Model V microcentrifuge (VWR scientific). The supernatant was then aspirated and the pellet resuspended in 1 ml PBS/1% BSA buffer plus 1:10 trypan blue. Escape velocity measurements were then taken of the cells. FIG. 54 illustrates the average escape velocity of the control cells as well as the camptothecin and topotecan-treated cells. As seen in FIG. 54, the topotecan-treated cells show a larger shift in escape velocity as compared to the camptothecin-treated cells. This result is consistent with the known higher potency of topotecan. The data shows that Optophoretic analysis was able to detect specific inhibition of topoisomerase with a chemical analog of camptothecin.

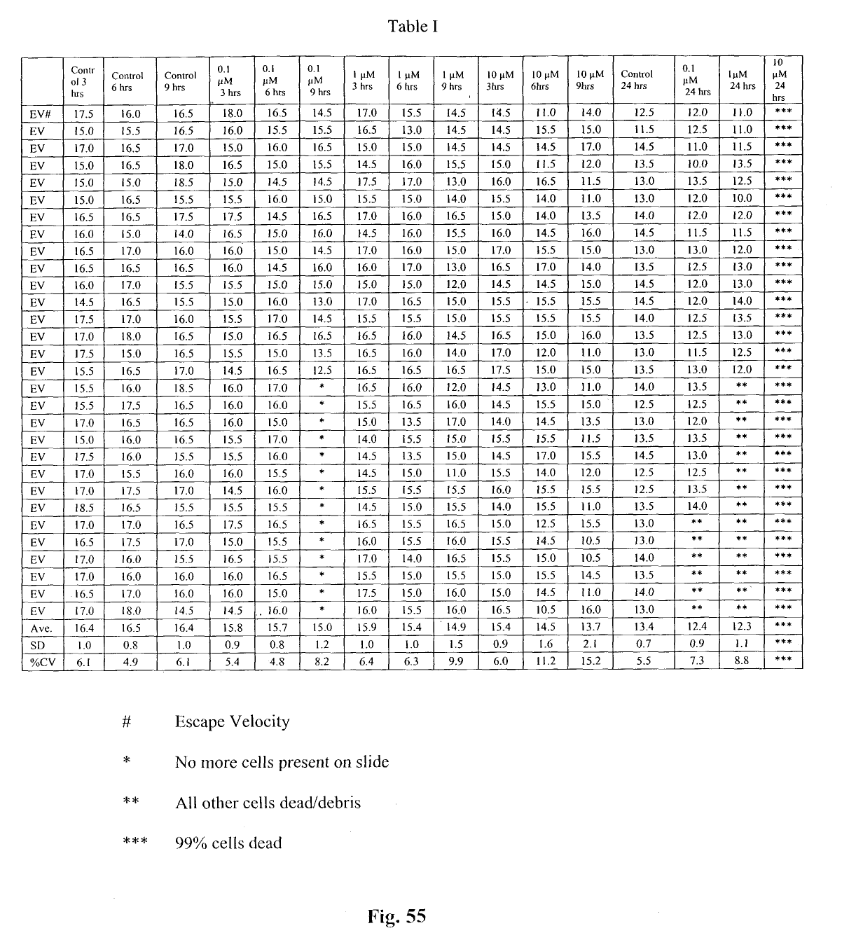

In a second experiment, U937 cells were incubated in media with varying concentrations of topotecan (0.1 μM, 1 μM, and 10 μM) dissolved to 0.1% DMSO. The different groups of cells were incubated for various time periods (3, 6, 9, and 24 hours) and escape velocity measurements were made. At the end of the incubation period, the cells were centrifuged down for five minutes at 5000 rpm. The supernatant was then removed and the pellet resuspended in 1 ml PBS/1% BSA buffer plus 1:10 trypan blue. The cells were then loaded onto a cover slip containing agarose that was spin-coated onto the surface thereof. Escape velocity measurements were then taken using a 100 mW laser with a diameter of 28 pixels (for the 24 hour measurements the diameter of the laser was 21.5 pixels. FIG. 55 shows the escape velocity measurements taken at the 3, 6, 9, and 24 hour time periods for the various concentrations.

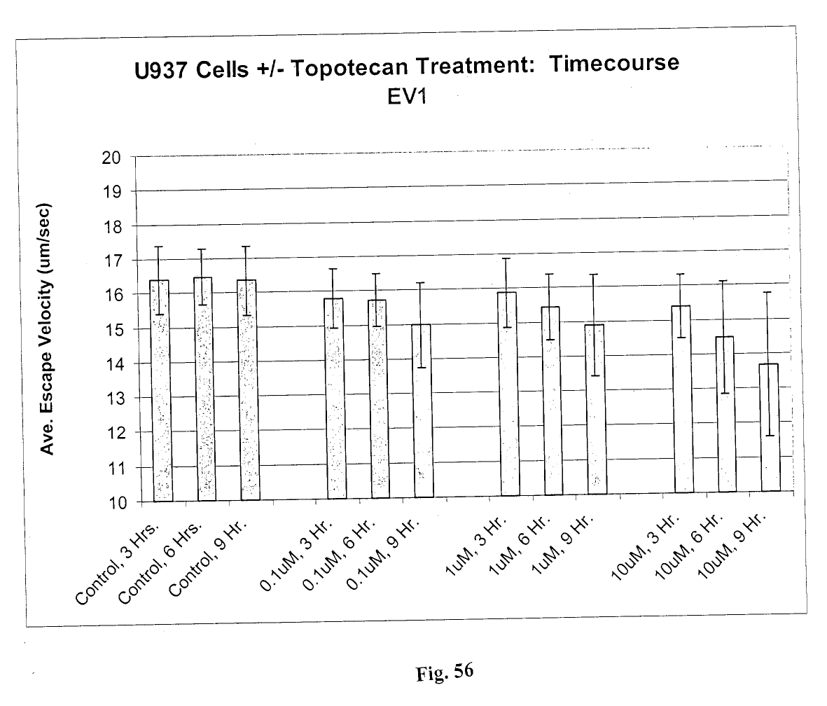

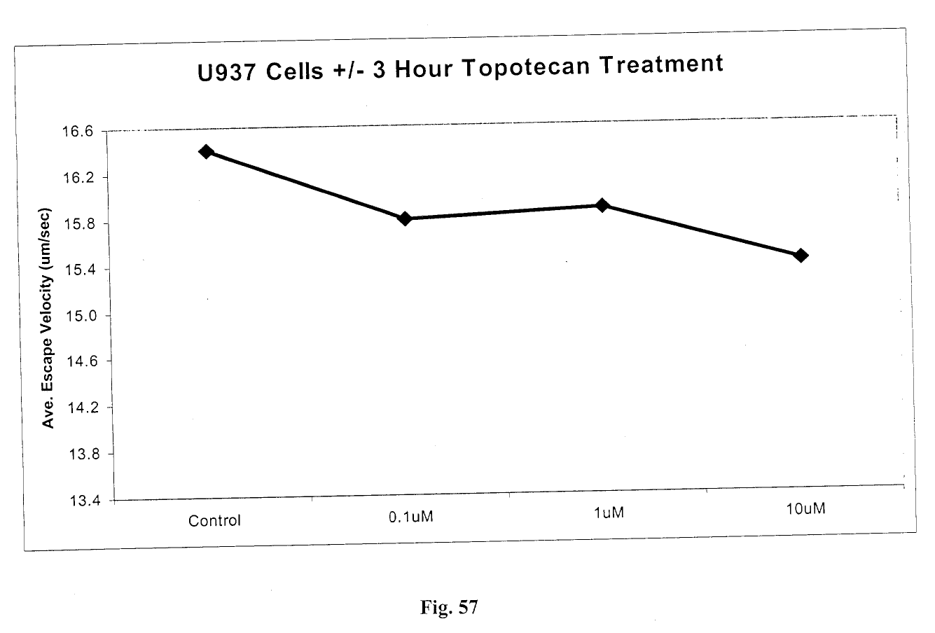

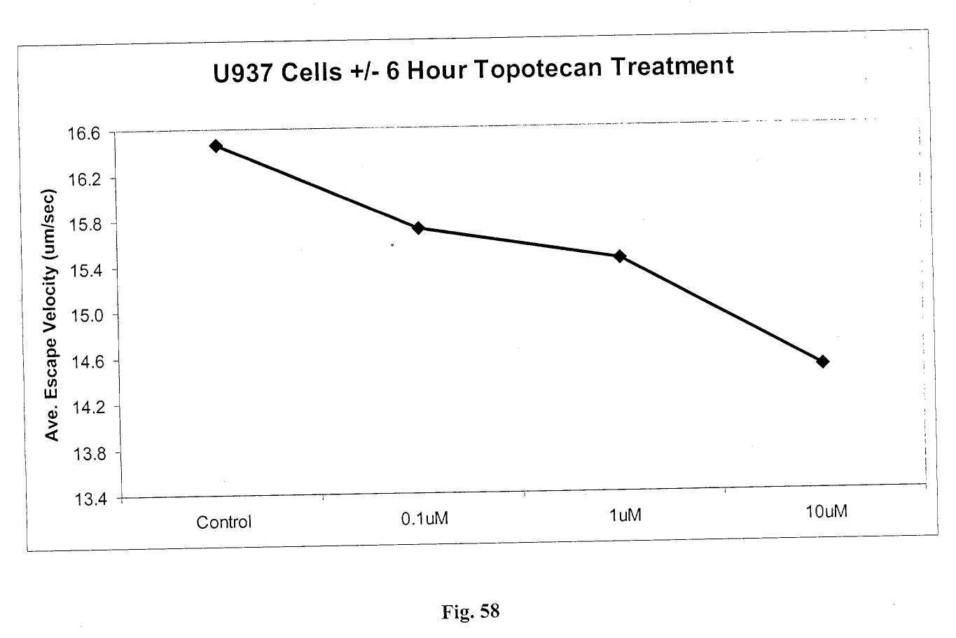

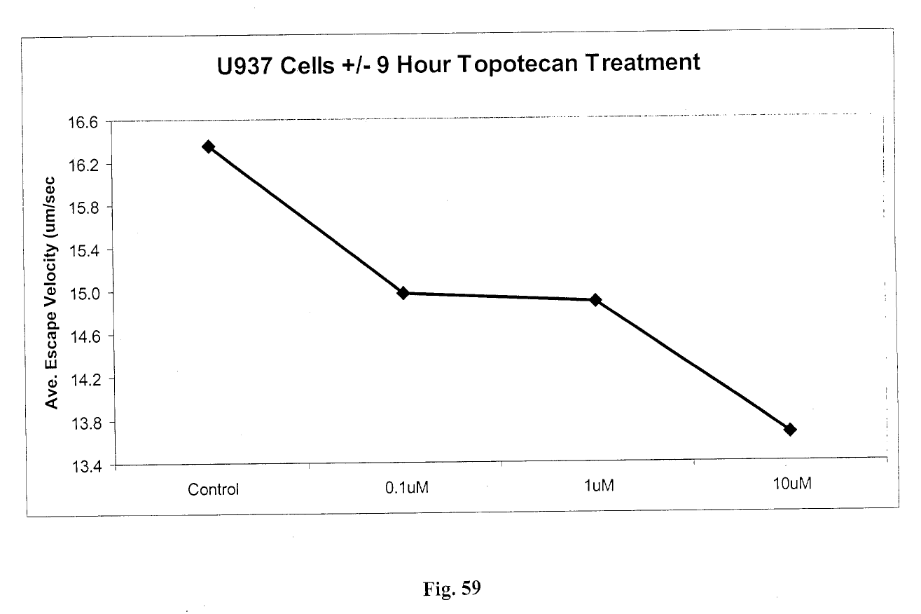

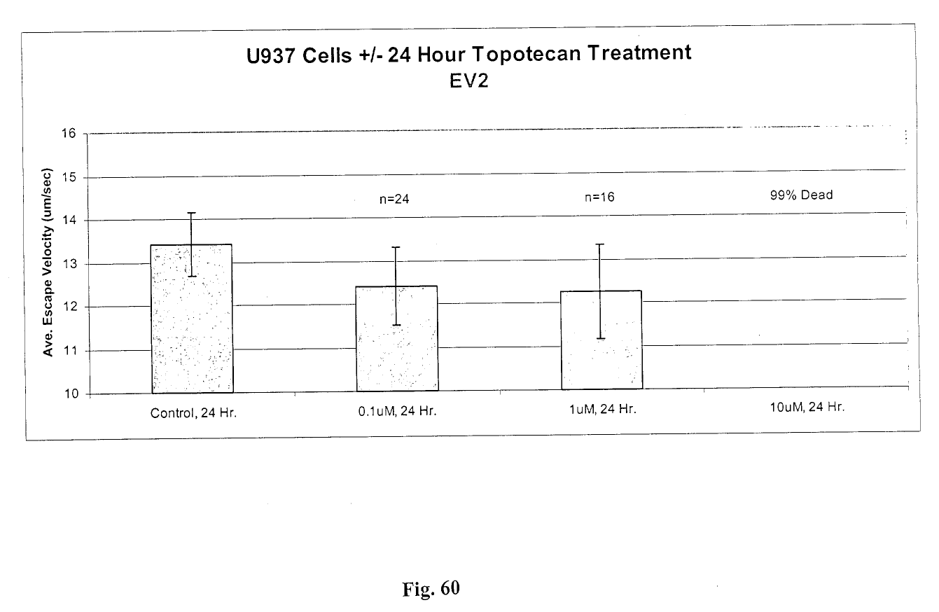

FIG. 56 illustrates the average measured escape velocities of the control and topotecan-treated cells at 3, 6, and 9 hours and at 0.1, 1, and 10 μM topotecan. FIGS. 57, 58, and 59 graphically illustrate the time course drop in measured escape velocities for the 0.1 μM, 1 μM, and 10 μM at the 3, 6, and 9 hour incubation time periods. As a general observation, the steepness of the curve increases with increasing incubation time periods. In addition, the escape velocity decrease increases as the concentration of topotecan increases. FIG. 60 illustrates the measured escape velocities at the 24 hour time period. For the cells treated with 10 μM topotecan, no data points were available because virtually all of the treated U937 cells were dead.

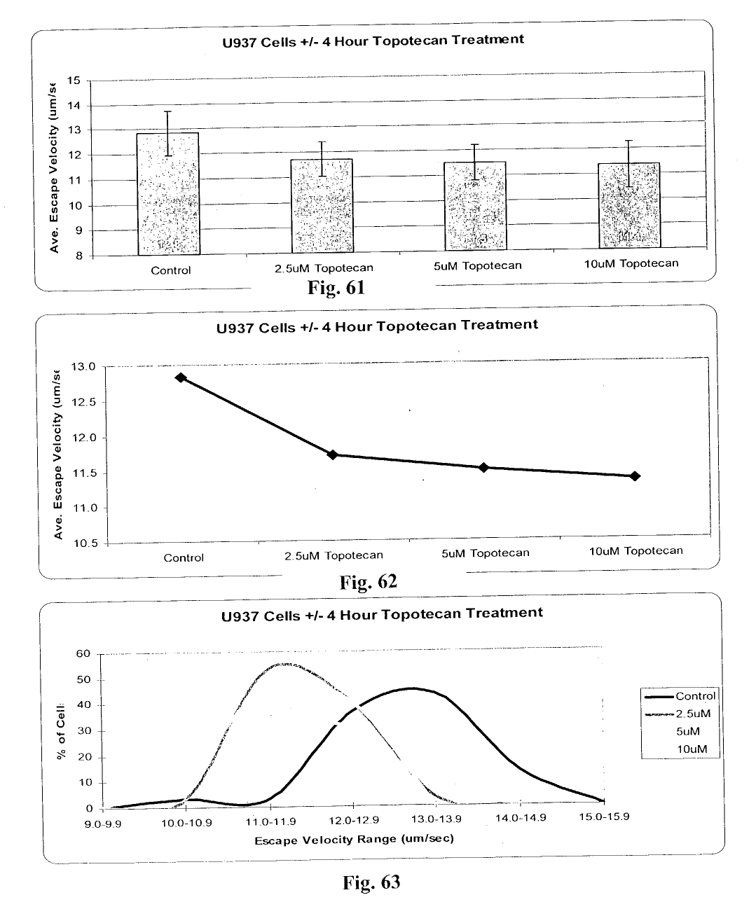

In a third experiment, concentrations of 2.5 μM, 5 μM, and 10 μM topotecan were tested on U937 cells. The cells were incubated for four hours and prepped for testing in accordance with the procedures described above in the prior experiments. The cells were then loaded onto a cover slip containing agarose that was spin-coated onto the surface thereof. Escape velocity measurements were then taken using a 100 mW laser with a diameter of 21.5 pixels. FIGS. 61 and 62 illustrate the average escape velocities of the control sample as well as the three concentrations. FIG. 63 illustrates the distribution of U937 cells as a function of escape velocity 4 hours after application of the topotecan. At the 4 hour mark, there were not a significant number of dead or necrotic cells.

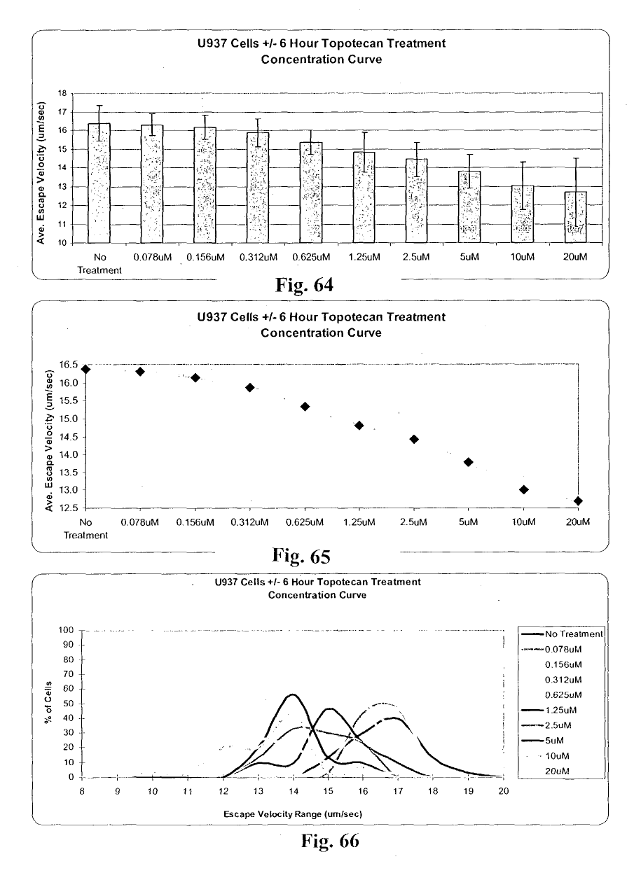

Based on the second and third experiments, 6 hours of incubation was chosen as the optimal balance between cell necrosis and a robust optophoretic response. In a fourth experiment, a full topotecan concentration curve was generated. Several samples of U937 cells were incubated with varying concentrations of topotecan dissolved to 0.1% DMSO. The groups of cells were incubated (or simply 0.1% DMSO in the case of the control) for six hours. At the end of the incubation period, the cells were centrifuged down for five minutes at 5000 rpm. The supernatant was then removed and the pellet resuspended in 1 ml PBS/1% BSA buffer plus 1:10 trypan blue. The cells were then loaded onto a cover slip containing agarose that was spin-coated onto the surface thereof. Escape velocity measurements were then taken using a 100 mW laser with a diameter of 28 pixels. Escape velocity measurements were then taken of the cells. FIGS. 64 and 65 illustrates the average escape velocity of the control cells as well as the topotecan-treated cells after the six hour time period. FIG. 66 shows the distribution of U937 cells as a function of escape velocity six hours after application of the topotecan. It was observed that at the higher concentrations, e.g., 10 μM and 20 μM, a significant amount of cellular debris was present.

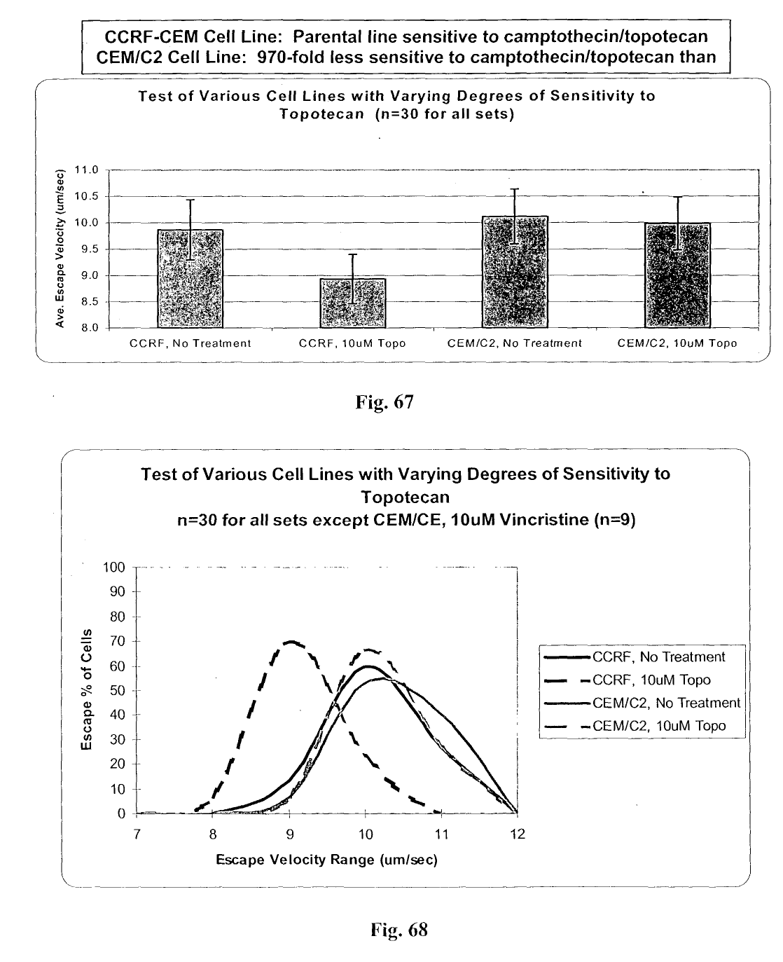

In a fifth experiment, two different cell lines obtained from ATCC which have varying degrees of sensitivity to topoisomerase I inhibitors such as camptothecin and topotecan were subject to Optophoretic analysis. A group of cells from a parental cell line CCRF-CEM that is sensitive to camptothecin and topotecan were incubated for 6 hours with 10 μM topotecan. Another group of cells from the cell line CEM/C2, which are 970-fold less sensitive to camptothecin and topotecan, were also incubated for 6 hours with 10 μM topotecan. Control cells from both the CCRF-CEM and CEM/C2 cell lines were also tested. The cells were incubated for six hours and prepared for testing in accordance with the procedures described above in the prior experiments. The cells were then loaded onto a cover slip containing agarose that was spin-coated onto the surface thereof. Escape velocity measurements were then taken using a 100 mW laser with a diameter of 28 pixels. FIG. 67 shows the mean escape velocities of the two cell lines in response to treatment with topotecan. FIG. 68 shows the distribution of U937 cells as a function of escape velocity range. As expected, the more sensitive CCRF-CEM cells showed a larger decrease in escape velocity as compared to the CEM/C2 cells. Optophoretic analysis was thus able to score cross-resistance of camptothecin-resistant cell lines to topotecan, an analog of camptothecin.

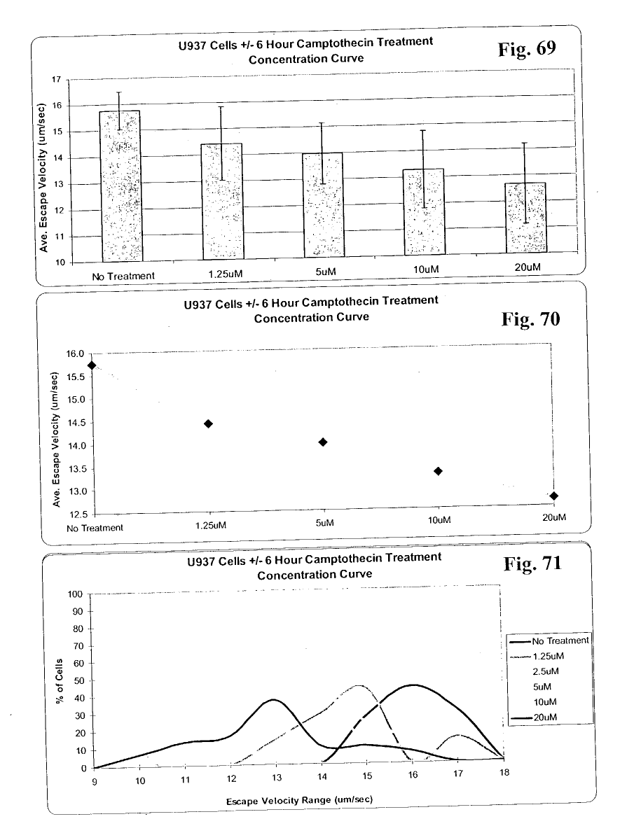

In a sixth and final experiment, a camptothecin concentration curve was generated. Several samples of U937 cells were incubated with varying concentrations of camptothecin (1.25 μM, 5 μM, 10 μM, and 20 μM) dissolved to 0.1% DMSO. The groups of cells were incubated for six hours with compound (or simply DMSO in the case of the control). At the end of the incubation period, the cells were centrifuged down for five minutes at 5000 rpm. The supernatant was then removed and the pellet resuspended in 1 ml PBS/1% BSA buffer plus 1:10 trypan blue. The cells were then loaded onto a cover slip containing agarose that was spin-coated onto the surface thereof. Escape velocity measurements were then taken using a 100 mW laser with a diameter of 19.5 pixels. Escape velocity measurements were then taken of the cells. FIGS. 69 and 70 illustrates the average escape velocity of the control cells as well as the camptothecin-treated cells after the six hour time period. FIG. 70 illustrates a lin-lin plot the escape velocity measurements of FIG. 69. FIG. 71 shows the distribution of U937 cells as a function of escape velocity six hours after application of the camptothecin. In comparing the data shown in FIG. 65 for topotecan and FIG. 70 for camptothecin, illustrate that Optophoretic analysis is able to quantitatively detect the known higher potency of topotecan as compared to camptothecin.

Optical Interrogation—Protein Expression Levels

Optical interrogation can also be used for the identification and selection of cells based on their protein expression levels. This is particularly important for biotechnology applications where living cells are used to produce proteins or other biopharmaceutical compounds. As stated in more detail above, cells are-typically grown in a bioreactor or similar device to produce the biopharmaceutical compound of interest. Optical interrogation can be used to monitor, for example, for quality control purposes, the environment and its impact on the cellular population. Moreover, optical interrogation can be used as an enrichment tool to retain the highest yielding cells while discarding the undesirable low yielding cells.

With respect to protein expression, a population of cells is provided that has a range of different expression levels of a specific protein. The population of cells is then subject to optical interrogation. The cells that have the desired expression levels (most often, the highest levels), are then segregated from the remaining cells. While the method has been described with respect to protein expression, the same steps can also be applied with respect to other biologically produced products.

The following are examples of various practical applications of optophoretic analysis.

Protein Expression

### EXAMPLE 1 (CHO-K1 CELL STUDY)

This experiment tested the escape velocities of two CHO cell lines: one normal, one containing a vector causing an over-expression of a G-coupled protein kinase receptor, specifically, the CCK-1-receptor. Both cell lines are first trypsinized using 3 ml trypsin/EDTA and incubated at 37° C. for 3 minutes. The cells were transferred to a conical tube and centrifuged at 500 rpm for 3 minutes. The cells were then washed with PBS. The cells were then resuspended in 5 mM EDTA. 10 ml of the sample was added to 20 ml of assay buffer/EDTA and 30 ml of trypan blue. The sample was placed onto a slide and inserted into an optophoretic system for measurement of escape velocities.

The following data was collected:

TABLE 10

| | Escape Velocity (μm/sec) | | |
|---|---|---|---|
| | | Standard | |
| Cell Line | Average | Deviation | % CV* |
| CHO-K1, Standard | 14.8 | 1.0 | 6.5 |
| CHO-K1, Protein | 16.3 | 0.6 | 3.5 |
| Expression | | | |

*CV denotes the coefficient of variation and is measured by the standard deviation divided by the mean.

*CV denotes the coefficient of variation and is measured by the standard deviation divided by the mean.

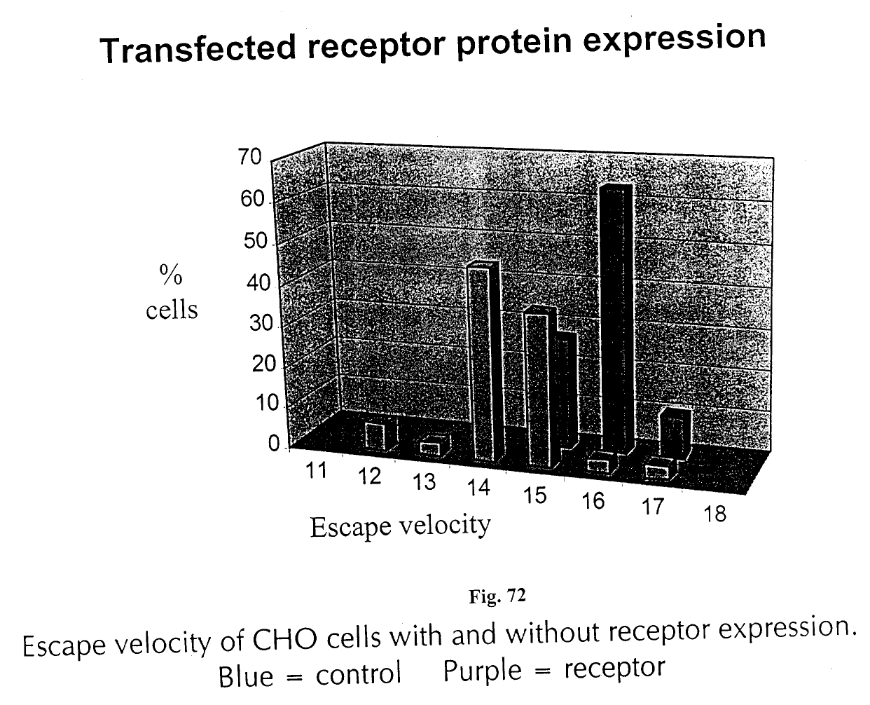

FIG. 72 shows the distribution of control and receptor-producing cells over a range of escape velocities. Cells which express the protein had an average escape velocity that was higher than that of the normal cells. In this manner, optical interrogation using Optophoresis is able to discriminate between cell lines based on their protein expression levels.

Protein Expression

### EXAMPLE 2 (CHO-K1 CELL STUDY)

An experiment was performed with three experimental cell lines and a control cell line of CHO cells. The experiment was to score clones of cell lines expressing varying levels of CCK-1 receptor using measured escape velocity, index match, and velocity modulation. A blind experiment was conducted to determine if optophoretic properties could distinguish low, medium and high expressing ranks for the clones. The clones were given identifiers of #11, #12, and #18.

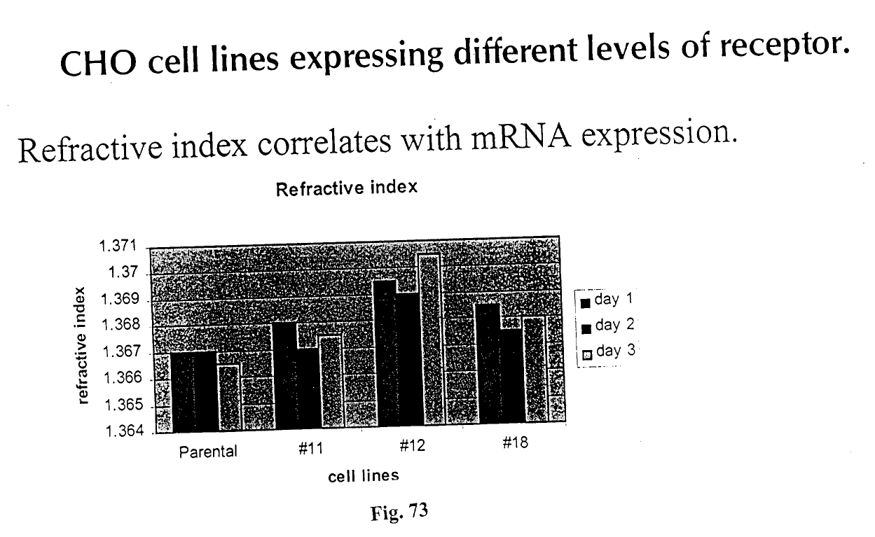

Escape velocities (μm/sec) were measured for three different test runs but did not show any particular trend in the various clones. The refractive index was measured for clones #11, #12, and #13 as well as the parental control of CHO cells. FIG. 73 shows the refractive index of these cells taken at over a period of three days. Independently, the mRNA levels of the clones and the parental control line were tested and the results agreed with the refractive index data shown in FIG. 73.

Protein Expression

### EXAMPLE 3 (SECRETION MODEL FOR B16 GM-CSF)

The objective of this experiment was to compare the escape velocities of cells secreting various levels of granulocyte-macrophage colony-stimulating factor (GM-CSF). The cell type used was mouse melanoma cells, B16.F10 that have been stably transfected with a plasmid construct containing the gene for GM-CSF. Three types of these cells were used which had varied levels of secretion of the protein. The B16.F10 wild type secretes no GM-CSF. B16.F10 sec 20 secretes a moderate level of GM-CSF, and B16.F10 sec 30 secretes the highest level.

TABLE 11

| | | Run 1 | Run 2 | Run 3 |
|---|---|---|---|---|
| B16.F10 | Average | 10.4 | 10.4 | 13.4 |
| wild type | SD | 0.6 | 1.0 | 1.0 |
| | % CV | 5.9 | 9.2 | 7.2 |
| B16.F10 sec | Average | 11.1 | 11.8 | 15.6 |
| 20 | SD | 0.5 | 0.9 | 0.9 |
| | % CV | 4.6 | 7.9 | 6.1 |
| B16.F10 sec | Average | 11.4 | 12.3 | 16.1 |
| 30 | SD | 0.7 | 0.6 | 1.3 |
| | % CV | 5.7 | 4.6 | 7.9 |

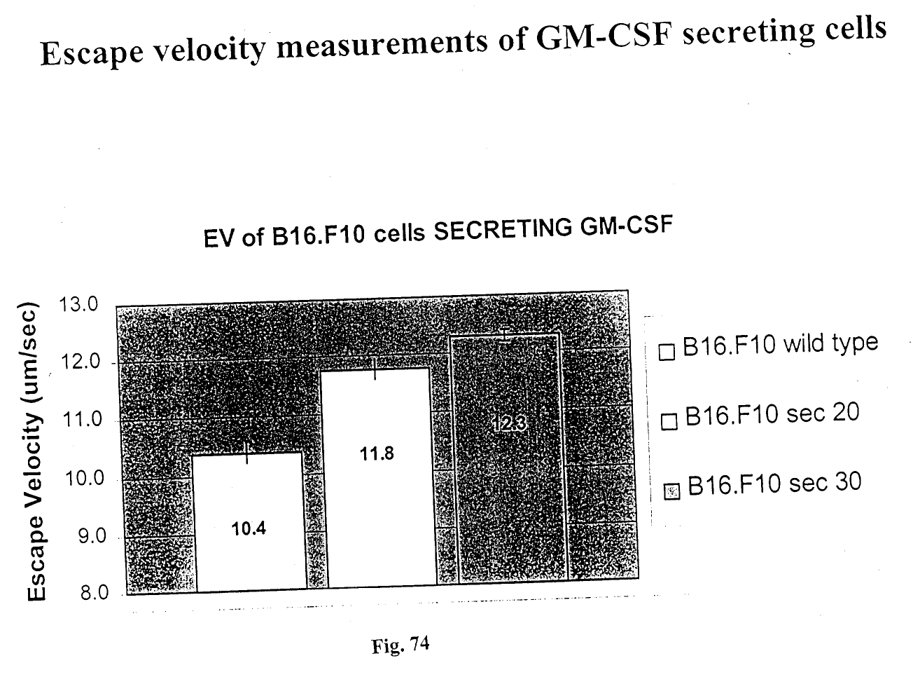

FIG. 74 shows escape velocity measurements of the three cell types, namely, B16.F10 wild type, B16.F10 sec 20, and B16.F10 sec 30. The data show that the higher producer (B16.F10 sec 30) had an increased escape velocity as compared to the moderate producer (B16.F10 sec 20) as well as the wild type (B16.F10 wild type) non-producer.

Optical Interrogation—Virus Detection

Optical interrogation can be used to determine whether a cell or group of cells are infected with a virus. In this method, a cell or group of cells is subject to optical interrogation wherein the cell(s) and the optical gradient are moved relative to one another. Cells that are infected with virus show a noticeable shift in escape velocity that becomes more pronounced with time of infection by the virus.

Viral Detection

### EXAMPLE 1 (TIME COURSE INFECTION OF 293 CELLS WITH ADENOVIRUS)

The experiment is designed to compare the optophoretic properties of cells containing adenovirus with the optophoretic properties of the same cells which have not been infected with the adenovirus. Another purpose of the experiment is to determine whether there are noticeable changes in escape velocity at various time points of infection. In this experiment, human embryonic kidney cells, HEK 293, were used. The virus used for infection was Ad5CMVGFP. Isolation of infected cells based on increasing levels of GFP expression was performed by flow cytometry.

The escape velocity of the cells was tested at various time points of infection. In addition, the relative fluorescence of the cells at the time points was measured with cytofluorometry. The time points used were no infection, 4 hours, 6 hours, 8 hours, 12 hours, and 24 hours post-infection. The Multiplicity Of Infection (“MOI”) for this experiment was 10. The results of this experiment are shown in the following table:

TABLE 12

| | No | | | | 12 | 24 |
|---|---|---|---|---|---|---|
| | virus | 4 hours | 6 hours | 8 hours | hours | hours |
| Escape | 10.7 | 10.9 | 11.3 | 11.2 | 12.1 | 12.0 |
| Velocity | | | | | | |
| Relative | 617 | 649 | 787 | 794 | 1174 | 4339 |
| Fluorescence | | | | | | |

A second run of this experiment was conducted. The MOI for the sample solution was about 31. After harvesting and rinsing, the cells were resuspended in 1% BSA PBS. Then a 1:2 dilution with trypan blue was performed, and the slide was made.

TABLE 13

| | No | | | | 12 | 24 |
|---|---|---|---|---|---|---|
| | virus | 4 hours | 6 hours | 8 hours | hours | hours |
| Escape | 10.2 | 11.2 | 11.4 | 11.7 | 12.1 | 12.4 |
| Velocity | | | | | | |
| Relative | 59 | 52 | 52 | 54 | 76 | 805 |
| Fluorescence | | | | | | |

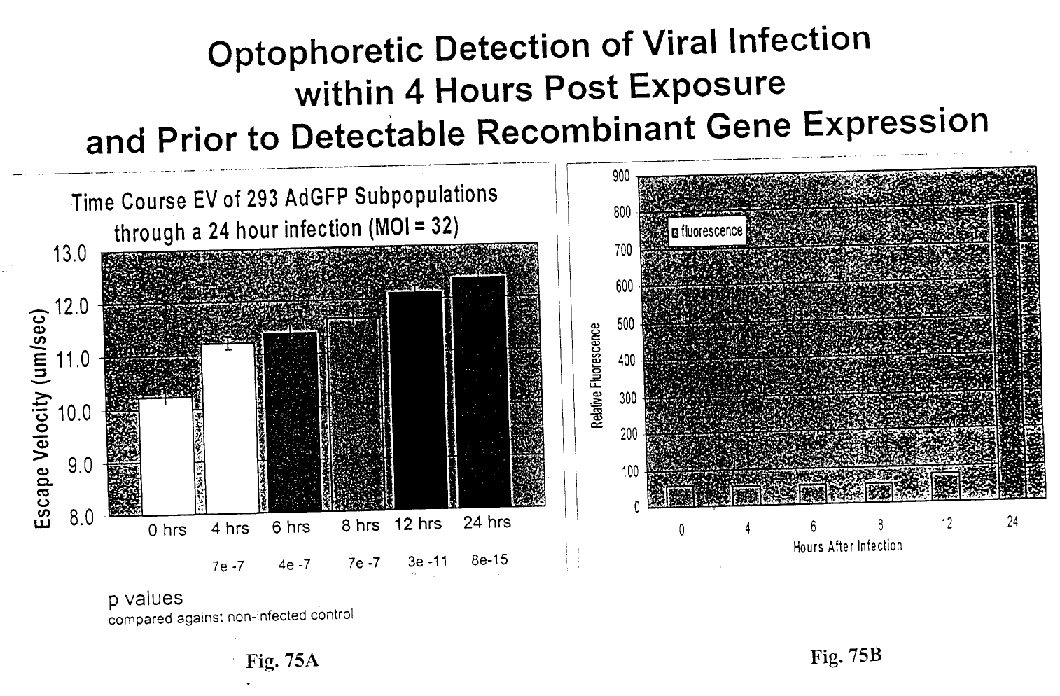

FIG. 75A shows the time course escape velocity data through 24 hours of infection. Noticeable changes in escape velocity are seen as early as 4 hours after infection. FIG. 75B shows the time course relative fluorescence of the cell population through the same 24 hours after infection. As seen in FIG. 75B, relative fluorescence changes in a significant manner only after 24 hours of infection.

Therefore, Optophoresis is able to detect infection in these cells as demonstrated by a time course shift in escape velocity. In addition, the effects of infection can be detected before a shift in relative fluorescence can be detected.

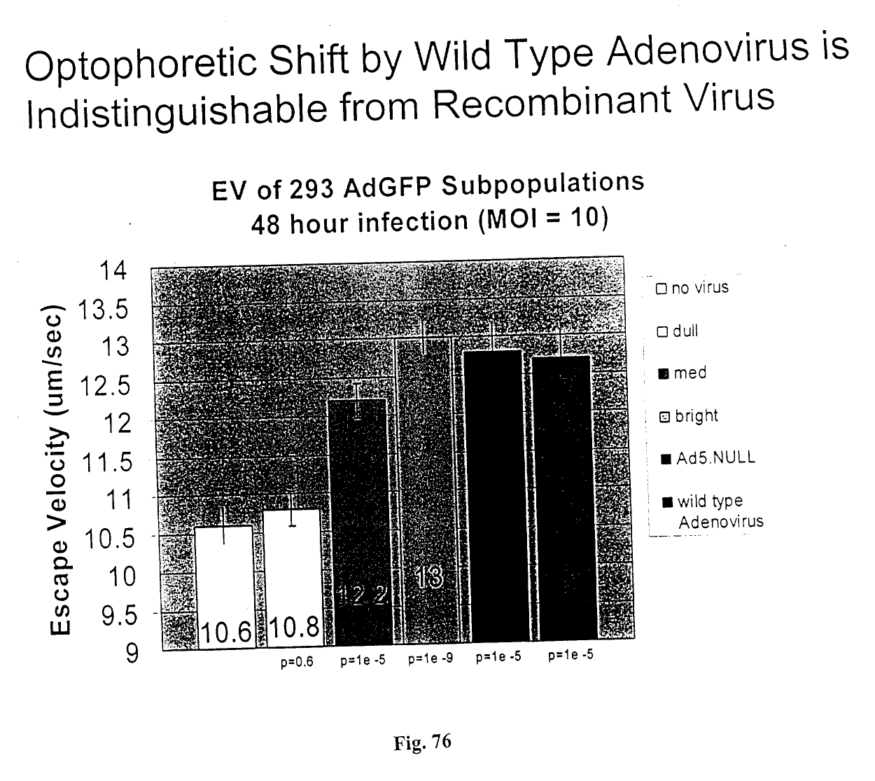

FIG. 76 shows the escape velocity of Adeno-GFP cells that have been infected with varying amounts of virus. Measurements were taken 48 hours after infection. The cells were divided into three groups, dull, medium, and bright. The brighter the fluorescence, the larger amount of virus contained within the cell. As shown in FIG. 76, escape velocity increased for cells having larger quantities of virus. In addition, in order to determine that the flourescent moiety was not responsible for the change in escape velocity values, the escape velocities of the wild type virus and the recombinant virus (no GFP) were tested. Based on the data, the optophoretic shift by the wild type adenovirus is indistinguishable from that of recombinant adenovirus. Consequently, it is the varying amounts of the virus and not the flourescent moiety that contributes to the change in escape velocities.

Viral Detection—Experiment 2 (Time Course Analysis of Adenovirus Infection of HeLa Cells)

The purpose of this experiment was to determine whether optophoretic properties can be used to distinguish between levels of infection in sample cells.

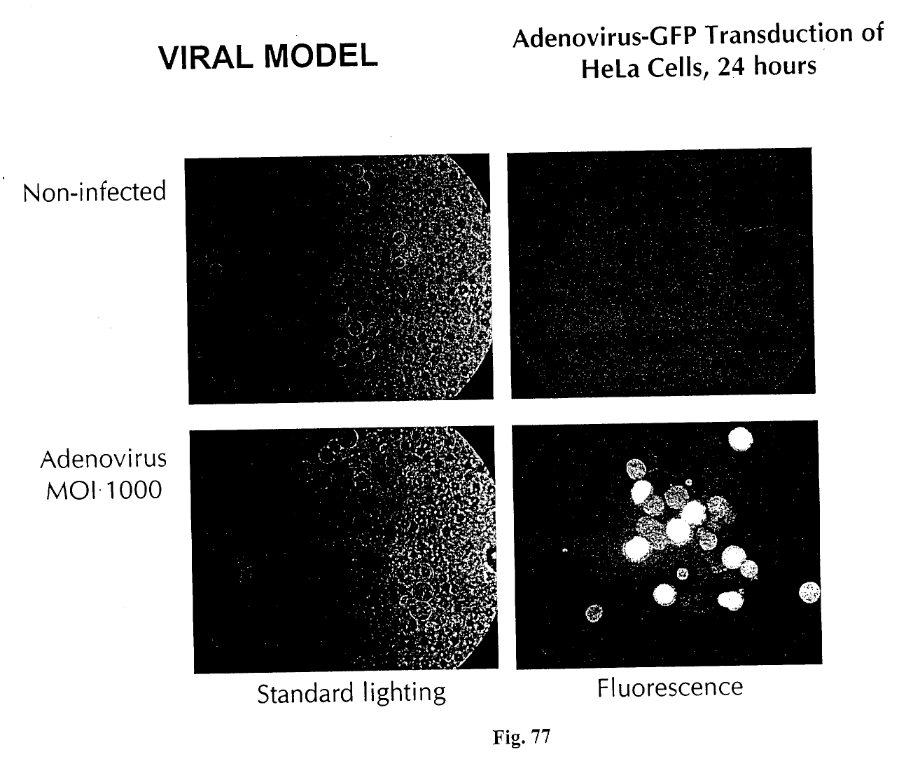

The sample cells used were HeLa cells which are human ovarian carcinoma cells. HeLa cells were infected with a virus (recombinant Adenovirus type 5 66 ElaΔE1BΔE3). The virus carried the transgene for GFP so that infectivity could be tracked. HeLa cells were transduced with 0, 30, 100, 300, 1000 MOI. FIG. 77 shows a panel of images of the infected and non-infected cells at 24 hours post-infection under fluorescence and standard lighting.

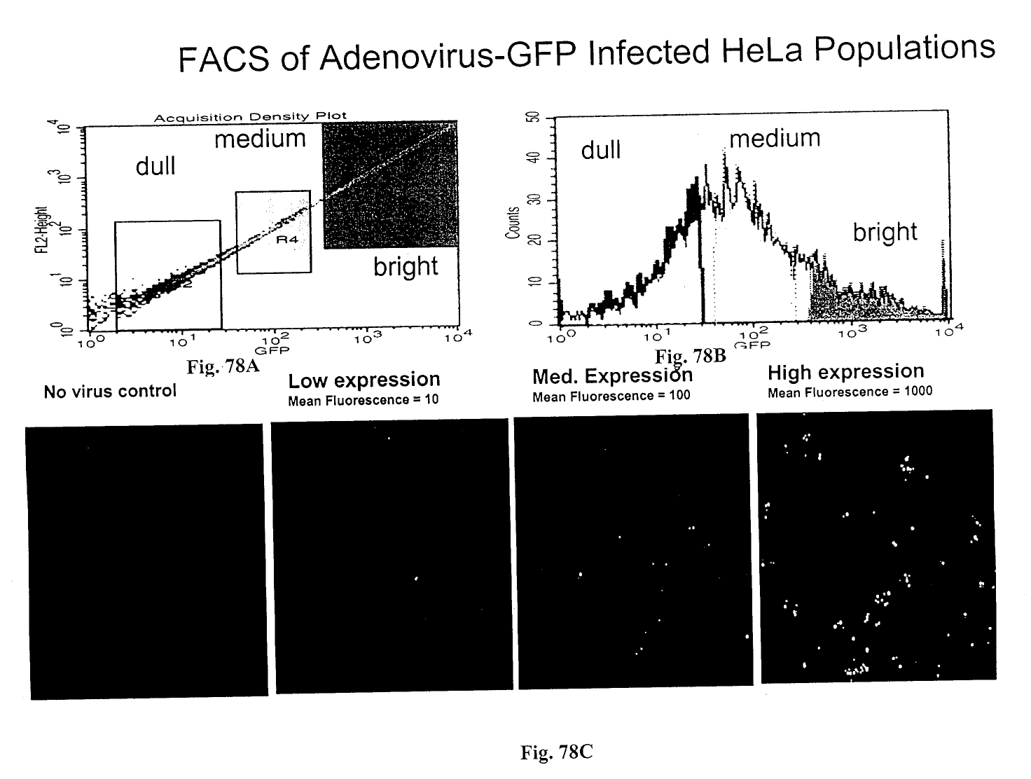

The infected cells were sorted using a Fluorescence-Activated Cell Sorter (FACS). FIG. 78A shows an acquisition density plot showing the three cell groups (dull, medium, and bright). The distribution of the infected cells is shown in FIG. 78B. FIG. 78C show images of the three cell groups including the non-infected control group. Transduced cells from the three highest MOI's were pooled after 48 hours and analyzed for escape velocity.

After 24 hours of exposure to the virus, GFP expression was observed indicating successful infection.

The results demonstrated a shift in escape velocity from 12.5 μm/sec in cells without the virus compared with 13.8 μm/sec in cells which were infected with the virus (n=30, p=0.0003). The level of fluorescence of the infected cells varied, some were weakly fluorescing and others were brightly fluorescing.

In a second portion of the experiment, cells were FACS sorted based on their level of fluorescence into dull, medium bright, and bright groups. The escape velocity of these cells were tested at 24 hours and 48 hours.

TABLE 14

| | 24 Hours - Run 1 | | | 24 Hours - Run 2 | | | 48 Hours | | |
|---|---|---|---|---|---|---|---|---|---|
| Cells (MOI = 300) | Ave. | SD | % CV | Ave. | SD | % CV | Ave. | SD | % CV |
| Non-transduced | 15.7 | 1.6 | 10.4 | 13.8 | 1.0 | 7.0 | 12.9 | 0.6 | 4.7 |
| Dull | 15.9 | 0.6 | 3.9 | 14.1 | 0.9 | 6.5 | 13.2 | 0.7 | 5.4 |
| Medium Briqht | 16.0 | 1.0 | 6.4 | 14.3 | 1.4 | 9.7 | 14.5 | 1.0 | 7.2 |
| Bright | 17.8 | 1.6 | 9.1 | 16.0 | 1.1 | 7.1 | 14.1 | 1.3 | 9.3 |

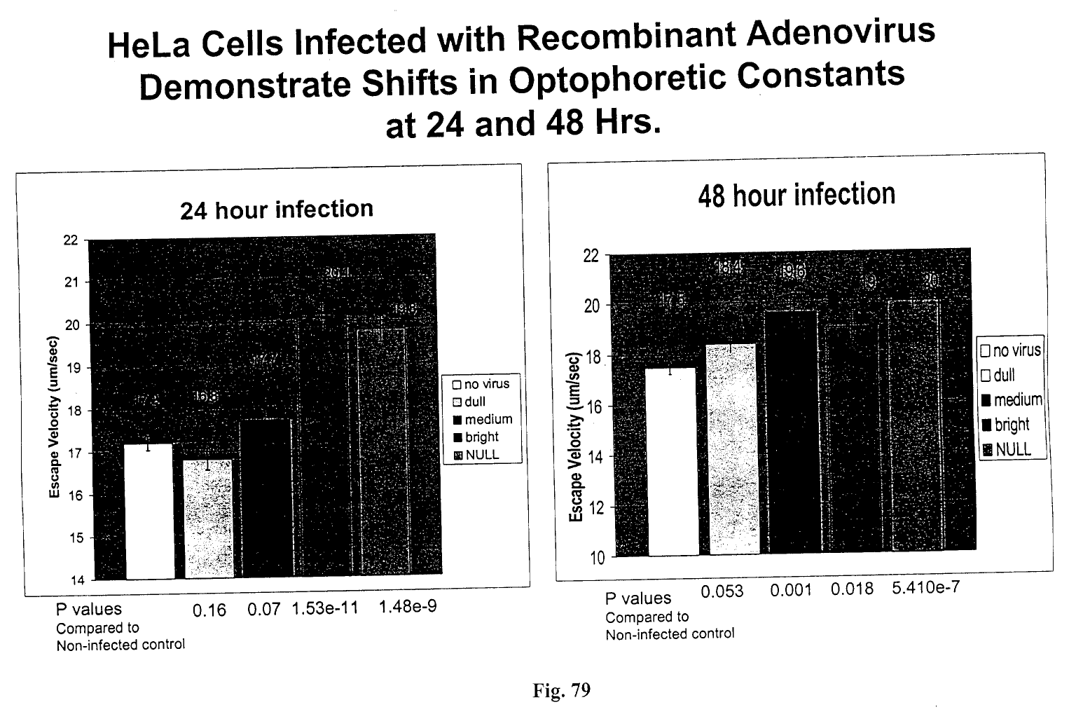

FIG. 79 graphically illustrate the result of another experiment on HeLa cells infected with recombinant adenovirus at 24 and 48 hours. Optophoretic shifts toward higher escape velocities can be seen at both 24 and 48 hours post-infection.

Infection of HeLa cells with recombinant adenovirus containing the gene for GFP show differences in escape velocity values at both 24 hours and 48 hours.

Viral Detection—Experiment 3 (K562 Cells)

The escape velocities of K562 AdGFP cells after 24 hours of infection with Ad-GFP with a MOI of 30 were:

TABLE 15

| | Run 1 | Run 2 |
|---|---|---|
| No virus | 12.0 | 14.4 |
| Unsorted | 10.7 | 15.4 |
| Bright | 13.2 | 13.9 |

Bacterial Screen For Drug Sensitivity—Experiment 1

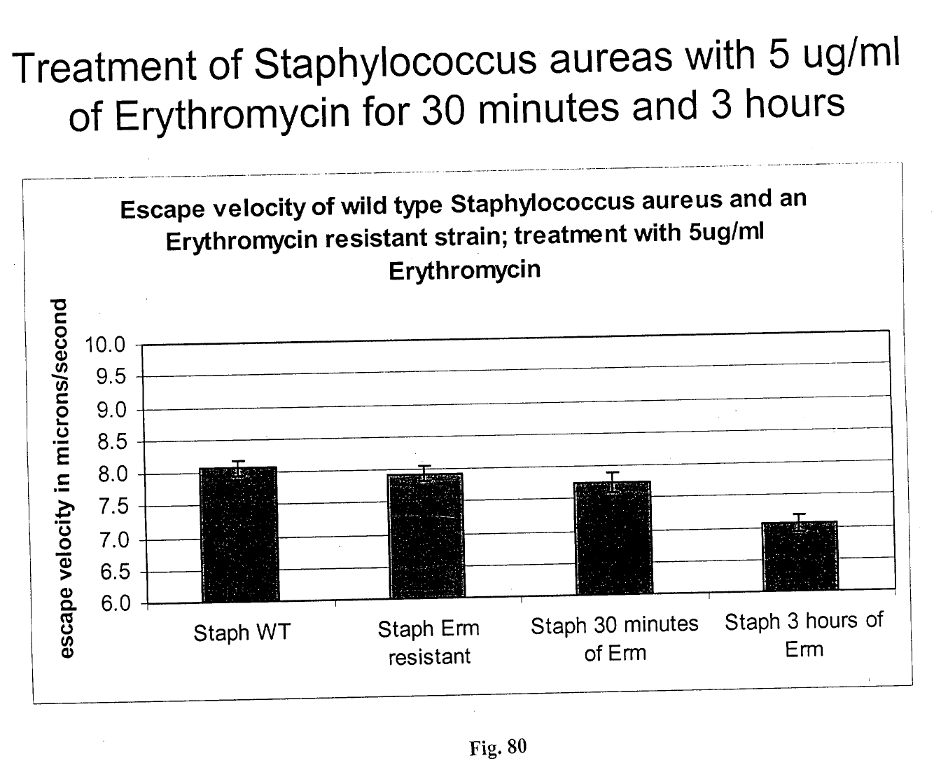

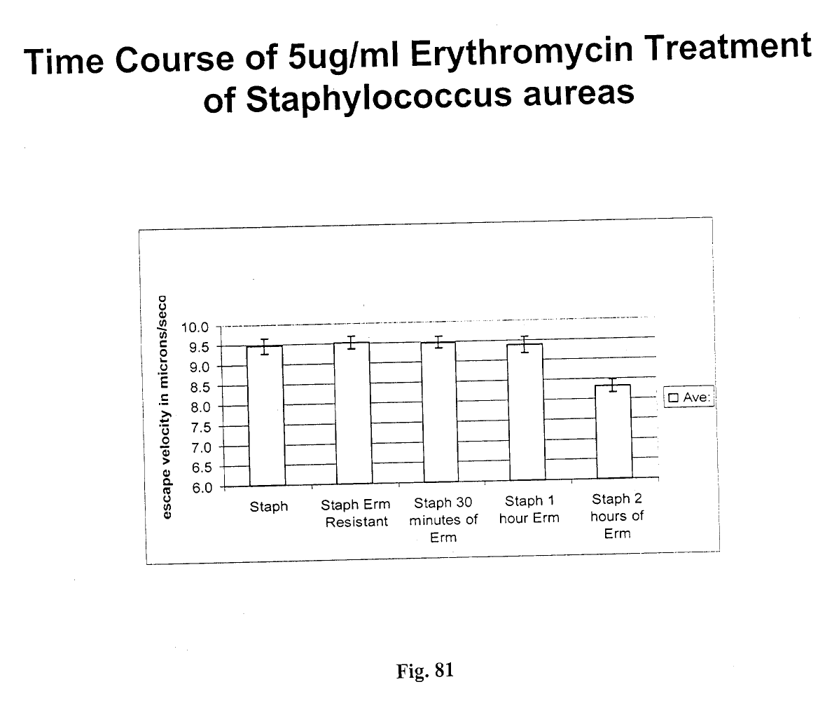

In this experiment, escape velocity was measured over time in wild type Staphylococcus aureus and an Erythromycin-resistant strain. 5 μg/ml of Erythromycin was applied to both the wild type Staphylococcus aureus and an Erythromycin-resistant strain. Escape velocity measurements were taken at time zero, 30 minutes post-treatment, and 3 hours post-treatment. FIG. 80 shows the changes over time in escape velocity of the Erythromycin-sensitive strain. As time progresses, escape velocity of the Erythromycin-sensitive strain decreases while the Erythromycin-resistant strain has the same escape velocity as the untreated Erythromycin-sensitive strain. FIG. 81 shows the results of another experiment in which 5 μg/ml of Erythromycin was applied to both the wild type Staphylococcus aureus and an Erythromycin-resistant strain. In this experiment, however, escape velocity measurements were made at time zero, 30 minutes post-treatment, and 1 hour post-treatment, and 2 hours post-treatment. A reduction in escape velocity of the Erythromycin-sensitive strain can be seen at 1 hour post-treatment.

Optical Interrogation of Wild Type/Mutant Yeast Strains

Experiment 1 (Escape Velocity)

In this experiment, the wild type Saccharomyces cerevisiae yeast (24657 rho(+)) strain and a mutant strain lacking mitochondrial DNA (MYA-1133 rho(0)) were subject to escape velocity and fast scan optical interrogation after 72 hours of growth. A 100 mW laser bean with a size of 14.2 mm was used to interrogate the respective strains. A seen in Table 16 below, there is a noticeable decrease in the escape velocity of the mutant MYA-1133 rho(0) strain.

TABLE 16

| | MYA-1133 (72 hrs.) | 24657 (72 hrs.) |
|---|---|---|
| * | Mutant | Wild Type |
| Ave. Escape Velocity | 31.7 | 39.5 |
| Standard Deviation | 3.6 | 4.5 |
| % CV | 11.3 | 11.5 |

* t-test = 7.6331E-10

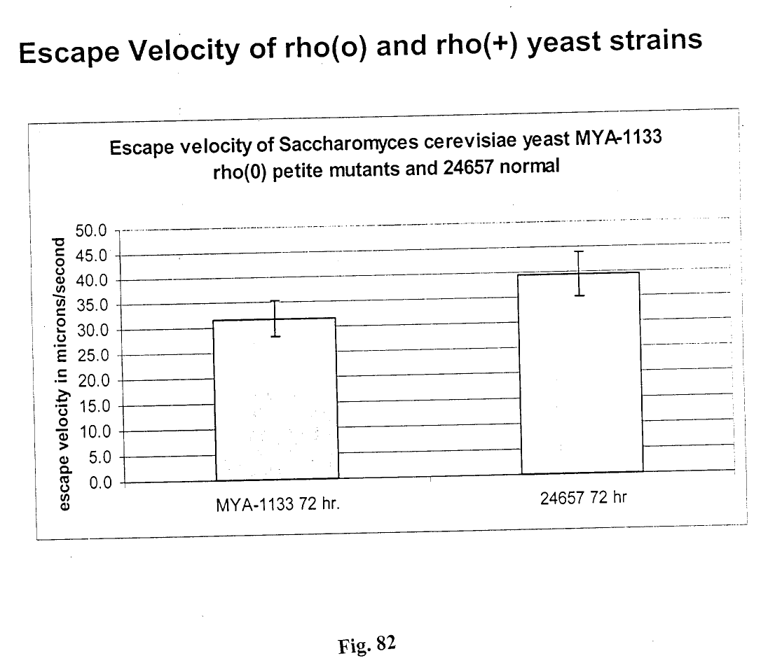

FIG. 82 graphically shows the escape velocity of the wild type and mutant strains.

Experiment 2 (Fast Scan)

A fast scan analysis was also performed on the wild type and mutant strains. In this experiment, a 173 mW laser beam was used with a scan speed rate of 20 μm/s to interrogate the two bacterial strains. The scan was repeated for six cycles. The average displacement values for the wild type and mutant strains were then measured. Table 17 reproduced below shows the results for the two strains.

TABLE 17

| | MYA-1133 rho(0) | 24657 rho+ |
|---|---|---|
| Average Displacement | 12.35 | 16.94 |
| Error | 1.27 | 1.14 |
| Std. Dev. | 6.96 | 6.22 |
| CV % | 56.33 | 36.73 |
| Data # | 30 | 30 |

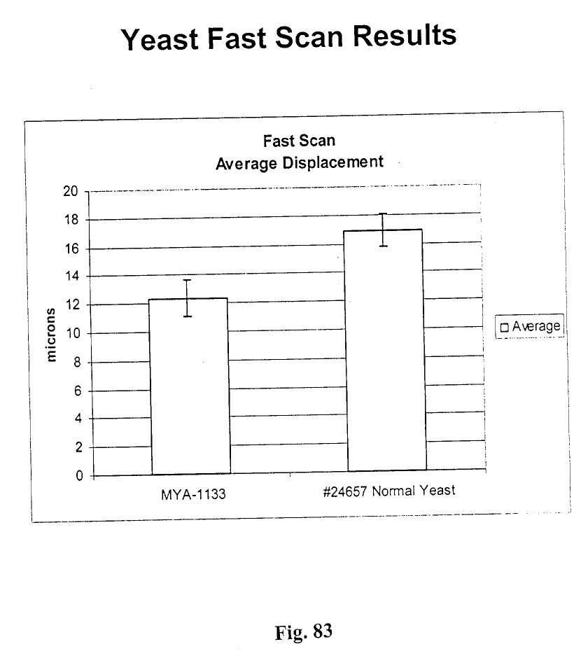

FIG. 83 graphically illustrates the results of the fast scan analysis. The data show that fast scan analysis can be used to discriminate between the mutant and wild type strains of yeast. The mutant strain has a lower average displacement as compared to the wild type.

Optophoretic Interrogation of Cells in Different Cell Cycle Stages

Experiment 1

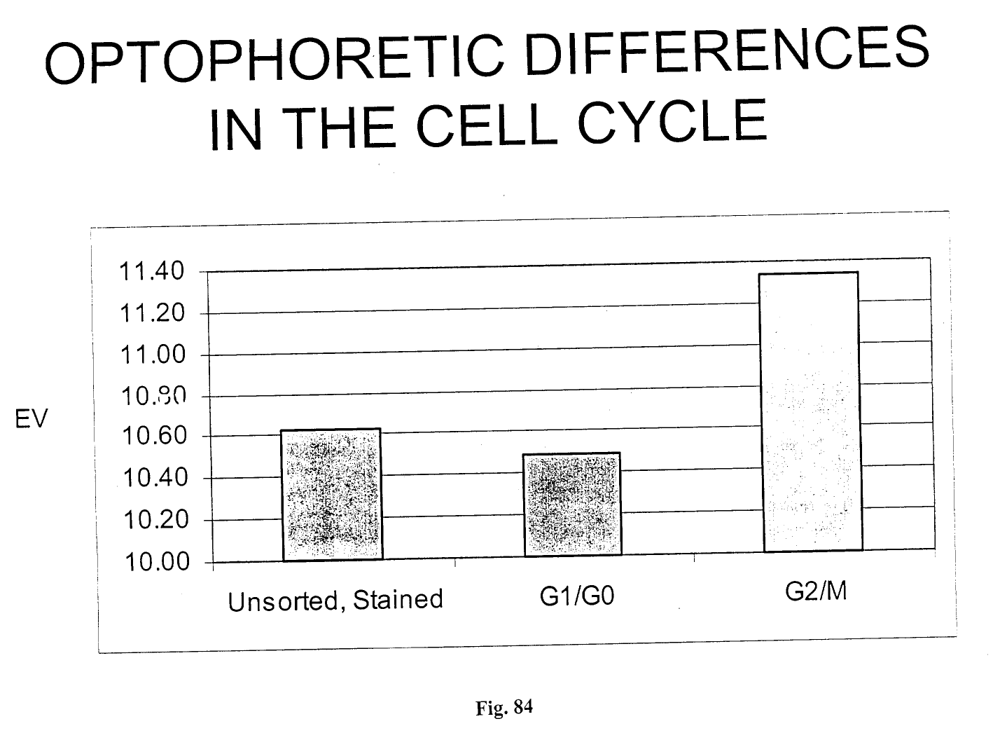

In this experiment, escape velocity was measured for cells that were in different stages of their cell cycle. Cells from an asynchronous rapidly growing cell culture population were analyzed and sorted using a fluorescent activated cell sorter to partition the population into two groups; those in G1/G0 which are not actively diving and those in G2/M which are in the process of active mitosis. These two purified populations were then subjected to analysis using Escape Velocity as was a sample of the original unsorted population of cells. FIG. 84 shows that the G1/G0 and G2/M values are distinct from one another and that the unsorted population has an escape velocity which is in between the values obtained for each of the sorted sub-populations.

Optical Interrogation of Live and Dead Microbes

Experiment 1—(Bacterium)

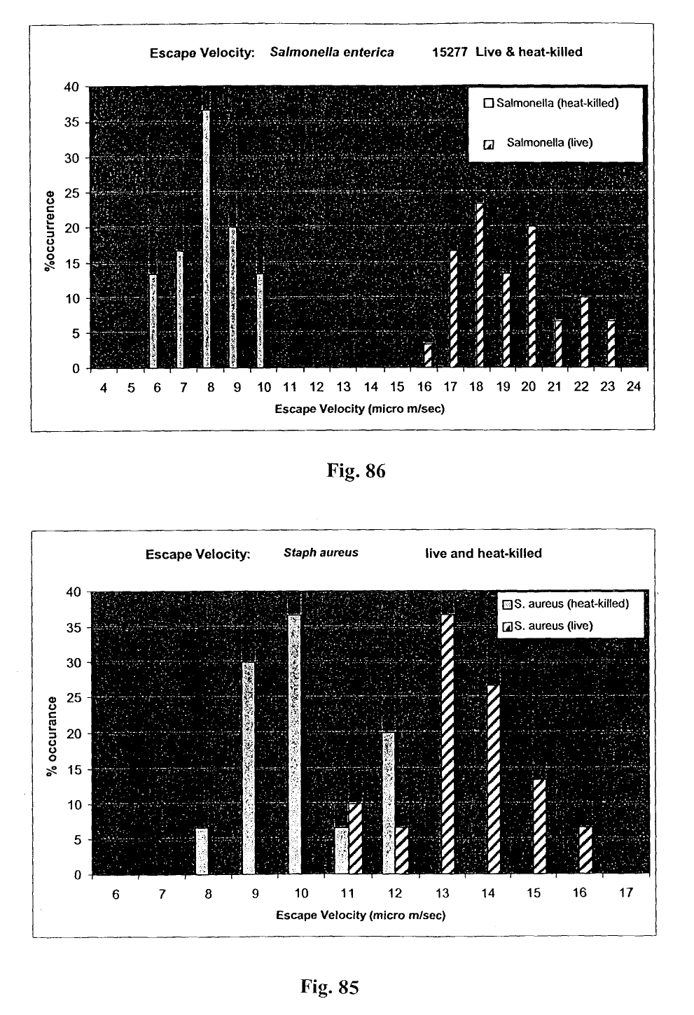

In this experiment, Optophoresis was used to interrogate live and dead (heat-killed) bacteria. A Gram positive bacterium, Staphylococcus aureus, was tested along with a Gram negative bacterium, Salmonella enterica. Cultures were prepared and grown of each strain of bacteria. A portion of each strain of bacteria was then rendered non-viable by heating at 95° C. for five minutes. Samples of the live and dead bacteria were then subject to optical interrogation by measurement of their respective escape velocities. FIG. 85 shows the distribution of escape velocities for live and heat-killed Staphylococcus aureus. The heat-killed bacteria generally show lower escape velocities. FIG. 86 shows the distribution of escape velocities for live and heat-killed Salmonella enterica. The heat-killed bacteria show lower escape velocities as compared to the live bacteria.

Optical Interrogation of Live and Dead Microbes

Experiment 2—(Yeast)

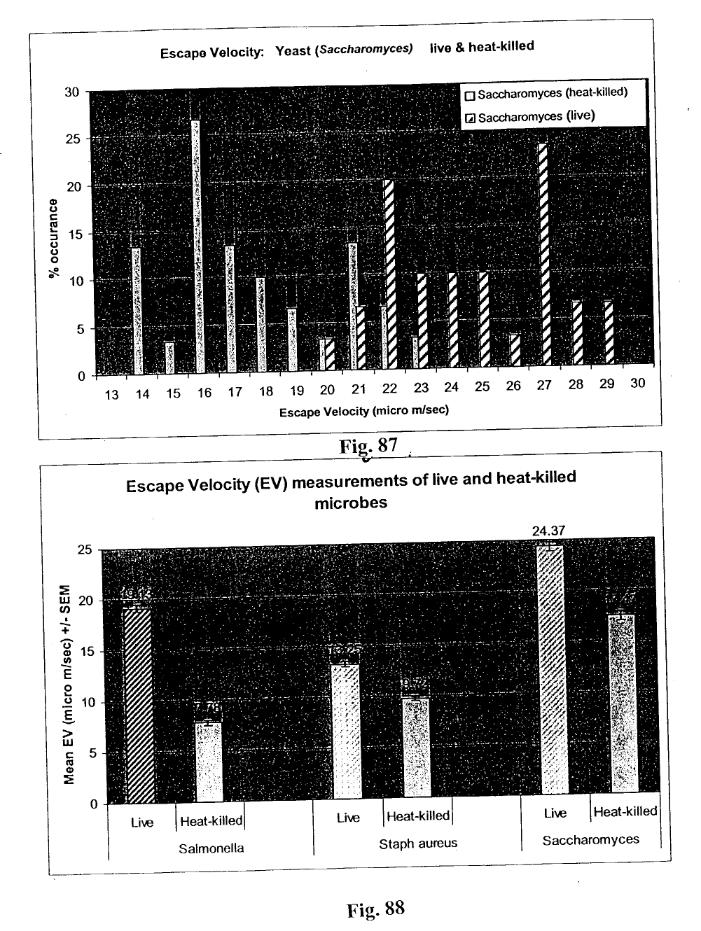

In this experiment, Optophoresis was used to interrogate live and dead (heat-killed) yeast. Saccharomyces cerevisiae was used as the strain of yeast. Cultures were prepared and grown and a portion was then rendered non-viable by heating at 95° C. for five minutes. Samples of the live and dead yeast were then subject to optical interrogation by measurement of their respective escape velocities. FIG. 87 shows the distribution of escape velocities for live and heat-killed Saccharomyces cerevisiae. The heat-killed yeast generally show lower escape velocities. FIG. 88 summarizes the results of experiments 1 and 2, showing the mean escape velocities for the live and heat-killed bacteria and yeast.

Early Detection of Apoptotic Events and Apoptosis using Optophoretic Analysis

Existing techniques and methods for the detection of apoptosis typically rely on the observation of marker events that are associated with the major pathways that trigger apoptosis. Many of the assays, however, are used detect events that take place in the later stages of apoptosis such as, for example, the development of leaking plasma membranes, nuclear breakdown, and chromosomal fragmentation. Assays for these events include staining with propidium iodide, or Hoeschst dye, and enzymatic or electrophoretic detection of fragmented DNA.

Still other assays provide earlier indications of the onset of apoptosis. For example, commercially available assays, such as the ApoAlert Annexin V assay (available from CLONETECH) are able to detect changes in the plasma membrane by using a FITC conjugate of annexin V. The ApoAlert Annexin V assay is based on the observation that within about 6-10 hours after the onset of apoptosis, most cell types translocate phosphatidylserine (PS) from the inner face of the plasma membrane to the exterior of the cell surface. Once the PS is exposed on the exterior of the cell surface, the presence of PS can be detected using a FITC conjugate of annexin V. Fluorescence microscopy and flow cytometry can then be used to detect the binding of the FITC conjugate to the exposed annexin V. While this assay does provide for relatively early detection of apoptotic events, the assay requires labeled annexin V markers as well as subsequent analysis by fluorescent microscopy or flow cytometry.

Another assay that provides for even earlier detection of apoptotic events are assays that detect caspase activation. For example, the Homogeneous Caspases Assay, Fluorimetric (available from Roche, catalog no. 3005372), is able to detect caspase activation. Caspases are autocatalytic proteases that are located at the upper end of the apoptotic proteolytic cascade and can thus be used for even earlier detection of apoptotic events (<4 hours) as compared to the Annexin V assay The above-identified kit detects caspase activation by assaying for the cleavage of a fluorescent substrate. The kit uses the substrate DEVD-Rhodamine 110 which is cleaved by activated caspases. When DEVD-Rhodamine 110 is cleaved, the released Rhodamine-110 molecule fluoresces upon excitation. By comparing the fluorescence from an apoptotic sample and an uninduced control, the increase in caspase activity can be quantified. While caspase assays do have the ability to detect relatively early apoptotic events, the assays require fluorometric enzyme substrates.

Optophoretic techniques have been investigated for their potential to detect the early onset of apoptosis in mammalian cells. U.S. patent application Ser. No. 10,240,611, for example, discloses multiple experiments in which U937 cells were treated with camptothecin and subsequently monitored Optophoretically using measured escape velocities. In one experiment, the escape velocity of the camptothecin-treated cells were measured at 4 and 6 hours after treatment. Noticeable changes in escape velocity were seen as early as 4 hours after treatment (see, e.g., FIG. 34). In that experiment, U937 cells were treated with 4 μg/ml of camptothecin and resuspended in PBS/1% BSA. Escape velocity measurements were taken after 4 and 6 hours. Table 18, reproduced below, includes the results of the escape velocity measurements for the test cells as well as the controls.

TABLE 18

| | | | U937 Cells | U937 Cells |
|---|---|---|---|---|
| | U937 Cells | U937 Cells | 4 hours -- | 6 hours -- |
| | (4 hour | (6 hour | 0.04 μg/ml | 0.04 μg/ml |
| | control) | control) | camptothecin | camptothecin |
| Escape | 11.52 | 11.79 | 10.16 | 9.52 |
| Velocity | | | | |
| Average | | | | |
| (μm/sec.) | | | | |

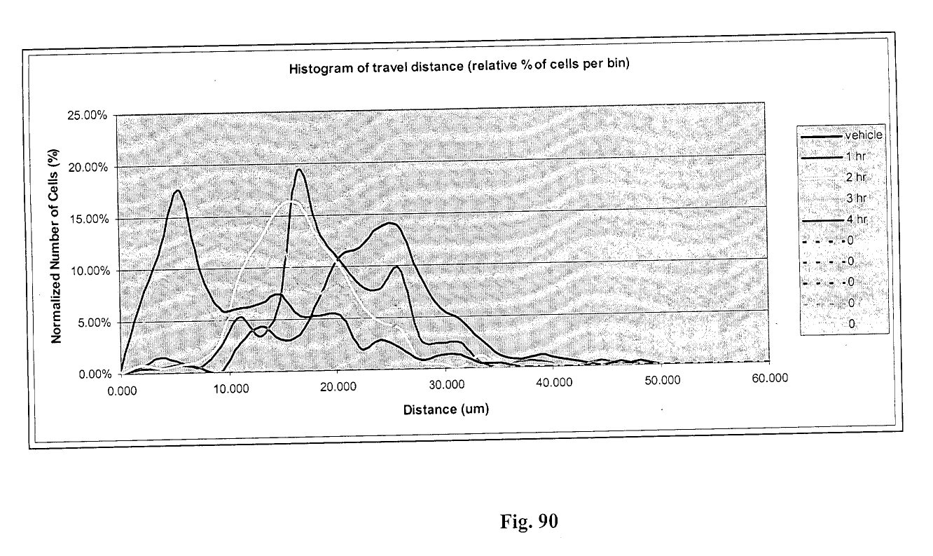

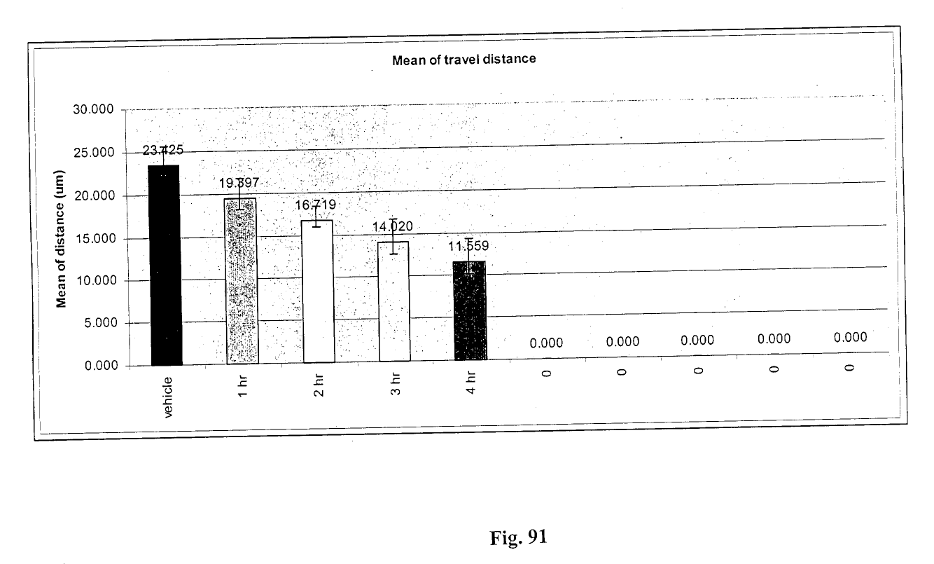

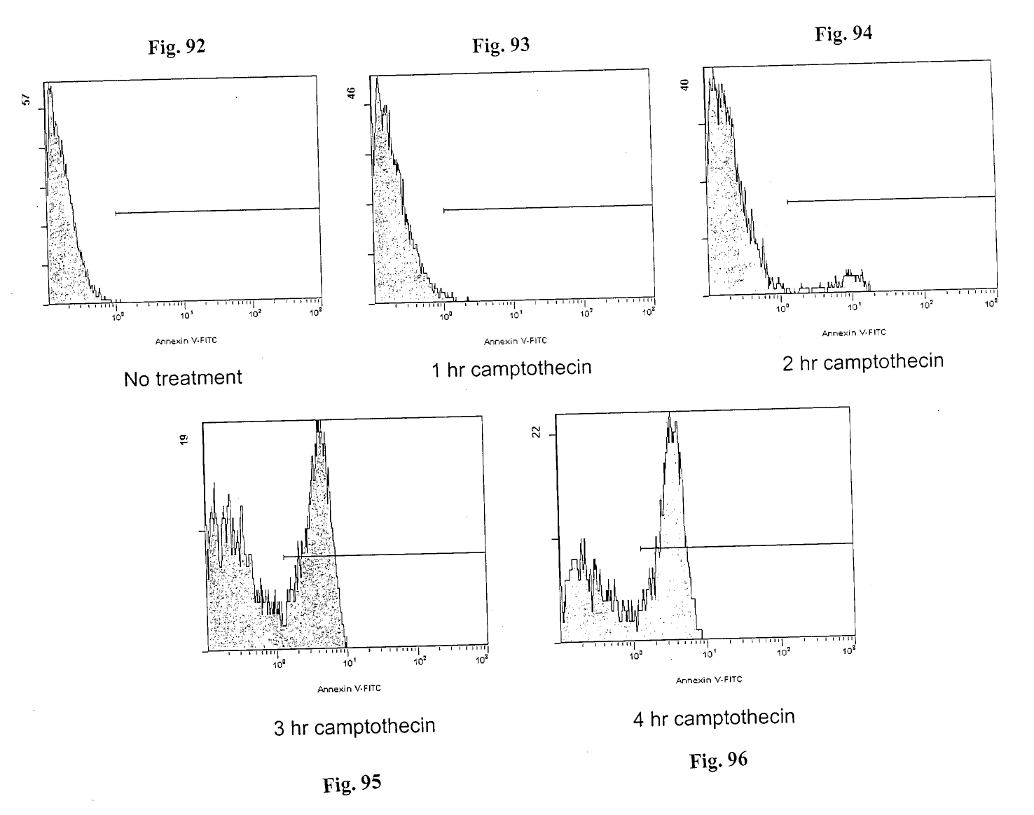

In another experiment, a fast scan analysis was performed on U937 cells that were treated with 4 μg/ml camptothecin. Measurements were taken of the treated cells at elapsed times of 1 hour, 2 hours, 3 hours, and 4 hours post-treatment. The control sample comprised U937 cells treated only with DMSO (solvent used for the camptothecin). Fast scan analysis was performed on an apparatus similar to that shown in FIG. 6. The travel distances of the treated and untreated cells were measured and recorded at each time interval. The total number of cells observed at each time interval ranged from 207 cells to 270 cells. FIG. 90 illustrates a histogram of the travel distances for treated and untreated U937 cells (vehicle) at the different time intervals. FIG. 91 illustrates the mean travel distances for cells at the control, 1 hour, 2 hour, 3 hour, and 4 hour time intervals. As seen in FIG. 92, a noticeably decrease in the mean travel distance is seen as early as 1 hour after administration of camptothecin.

Another experiment using U937 cells treated with 4 μg/ml camptothecin was also carried out. In this experiment, however, the cells were subject to analysis using an annexin V assay. The assay was performed using the ApoAlert Annexin V Apoptosis Kit (available from Clonetech, catalog no. K2025-1). FIGS. 92-96 shows a panel of five FACS graphs showing the cell number as a function of log annexin V binding for the control as well as the 1 hour, 2 hour, 3 hour, and 4 hour time intervals. As seen in FIG. 94, the annexin V assay begins to detect the onset of apoptosis at the 2 hour mark. Significant annexin V binding is seen later in the 3 and 4 hour time points. FIGS. 97-101 show the FACS annexin V profile of the treated and untreated U937 cells. Quadrant B4 shows a significant increase in cell count at the 2 hour mark (FIG. 99).

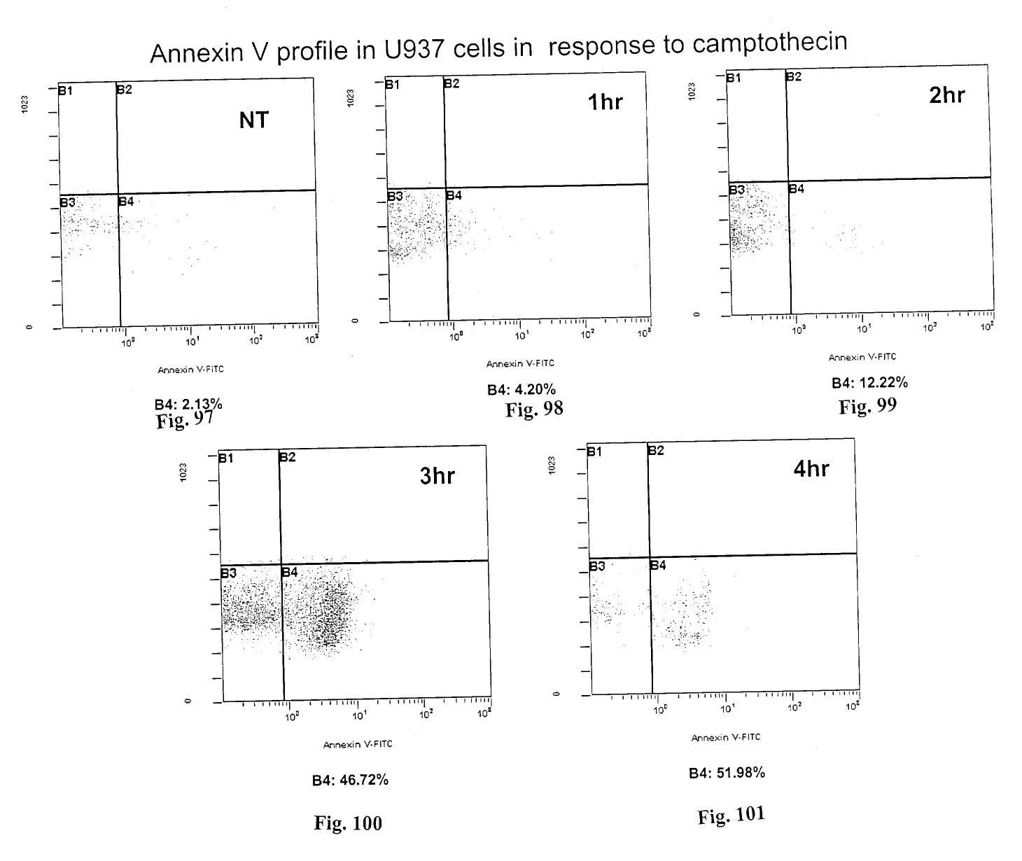

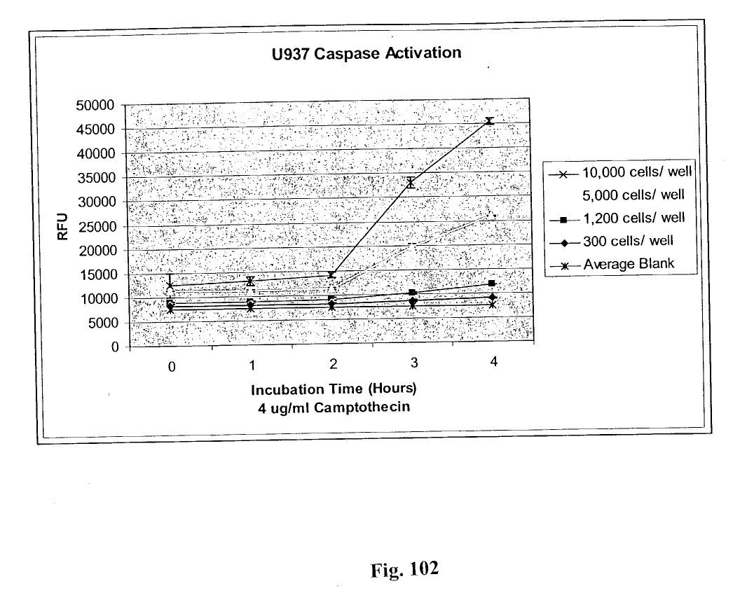

Yet another experiment using U937 cells treated with 4 μg/ml camptothecin was carried out. In this experiment, the cells were subject to analysis using a caspase assay (Homogeneous Caspases Assay, Fluorimetric-available from Roche, catalog no. 3005372). The caspase assay is a fluorometric assay carried out in 96-well plates. Treated cells were incubated with DEVD-Rhodamine 110 and treated with 4 μg/ml camptothecin. Upon cleavage of the substrate by activated caspases, fluorescence of the released Rhodamine 110 is measured. The experiment was carried out with different numbers of U937 cells contained per well (300, 1,200, 5,000, 10,000). FIG. 102 illustrates a graph of the relative fluorescence units (RFU) as a function of incubation times (hours) for the control (blank) as well as the camptothecin-treated cells. At the highest cell concentration level (10,000 cells/well), caspase activity is detected somewhere between 2 and 3 hours after administration. In the lowest cell concentration (300 cells/well), caspase activity is not detected even after 4 hours of camptothecin administration.

Optophoretic analysis of early apoptotic events has several advantages over the annexin V and caspase assays. First, Optophoretic analysis, such as fast scan analysis, obviates the need for laborious and expensive fluorometric enzyme substrates and markers. Second, Optophoretic analysis is able to detect apoptotic events earlier than convention assays such as the annexin V assay and caspase assays. This is particularly true for low concentrations of cells/well. Typically, caspase assays recommend that on the order of 40,000 to 100,000 cells/well be used in the assay. In contrast, by using Optophoretic analysis techniques, the onset of apoptosis in U937 cells was detected after about 1 hour of treatment with camptothecin. This analysis required less than 300 cells. Finally, Optophoretic analysis allows for the observation of apoptotic events from the very beginning of apoptosis to final apoptosis while the cells remain viable and unaltered by fluorescent markers and the like.

Detection and Evaluation of Cancer Cells using Optophoretic Analysis

Optophoretic interrogation has been used to distinguish cancer cells from normal cells for breast carcinoma and skin melanoma. In this regard, Optophoretic interrogation can be used as a diagnostic tool to determine whether cells show the Optophoretic characteristics of cancer cells or normal cells These provide a relatively quick way of diagnosing whether a sample contains cancerous cells. The technique advantageously may be used with relatively small sample sizes. The Optophoretic interrogation techniques may be used beyond breast cancer and skin cancer to other types of cancers including, but not limited to, colorectal cancer, lung cancer, prostate cancer, renal cancer, endometrial cancer, esophageal cancer, gastric cancer, bladder cancer, brain cancer, cervical cancer, testicular cancer, and pancreatic cancer.

Experiments have been conducted on human breast carcinoma cell lines as well as human melanoma cell lines. Tumor cell lines were purchased from ATCC and, when available, their normal counterparts were matched from the same patient. Cells were grown in culture until the time of testing. Adherent cells were detached from culture flasks using trypsin and resuspended in buffer. Cells were then subject to Optophoretic interrogation.

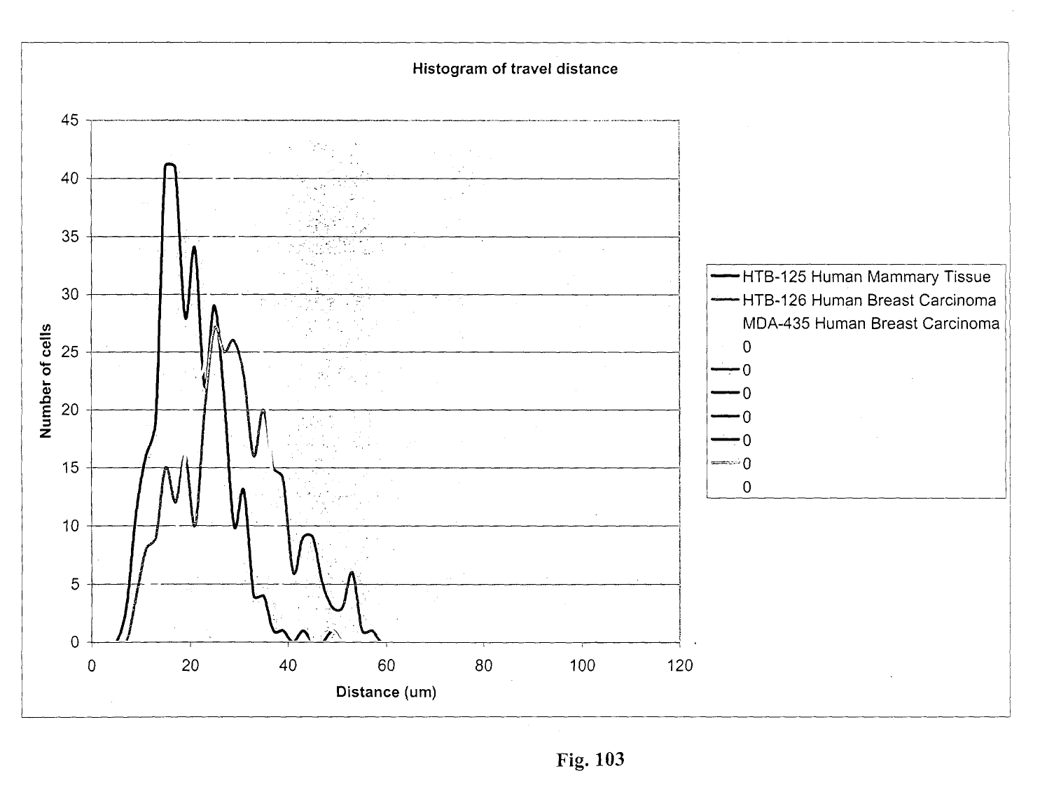

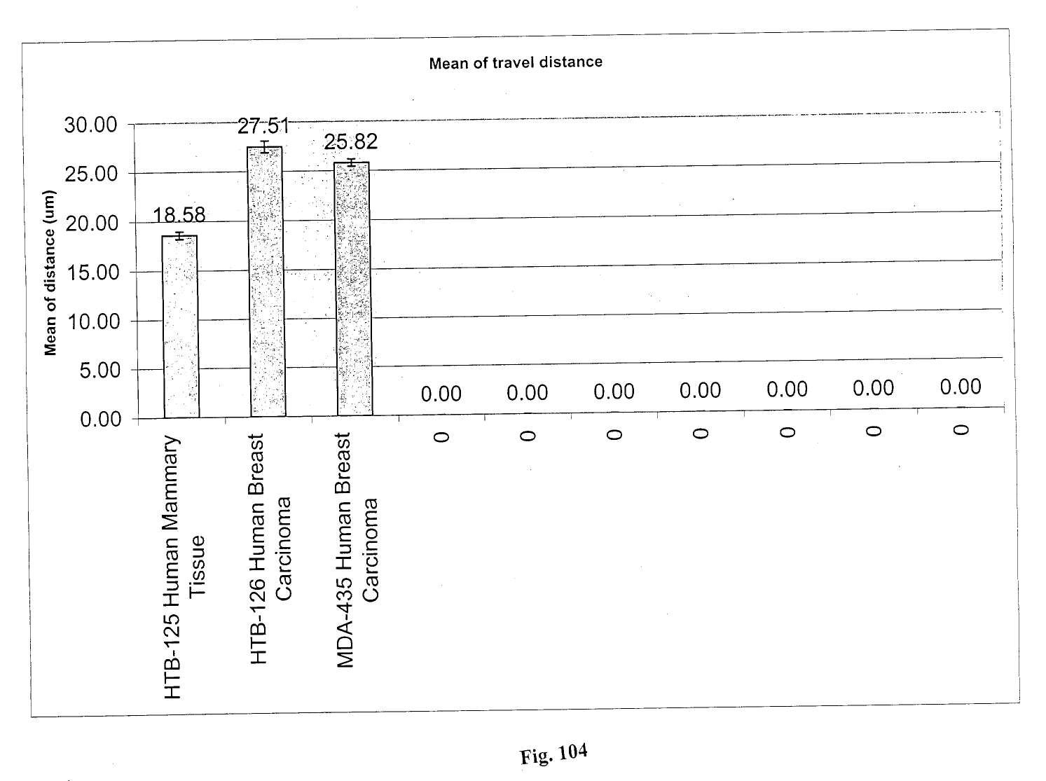

In one experiment, MDA-435 breast carcinoma cells were tested using fast scan analysis along with a matched sample of breast carcinoma cells (HS578T) and cells obtained from normal mammary tissue (HS578BST). FIG. 103 shows the histogram of travel distance of the three cell types. Both types of breast carcinoma cells (HS578T and MDA-435) show markedly higher travel distances as compared to the cells obtained form normal mammary tissue (HS578BST). In addition, with respect to the matched samples of cancerous and non-cancerous cells (HS578T and HS578BST), the HS578T cancer cells shows a significant increase in travel distance as compared to the non-cancerous HS578BST cells. FIG. 104 shows the mean travel distances for the three cell types.

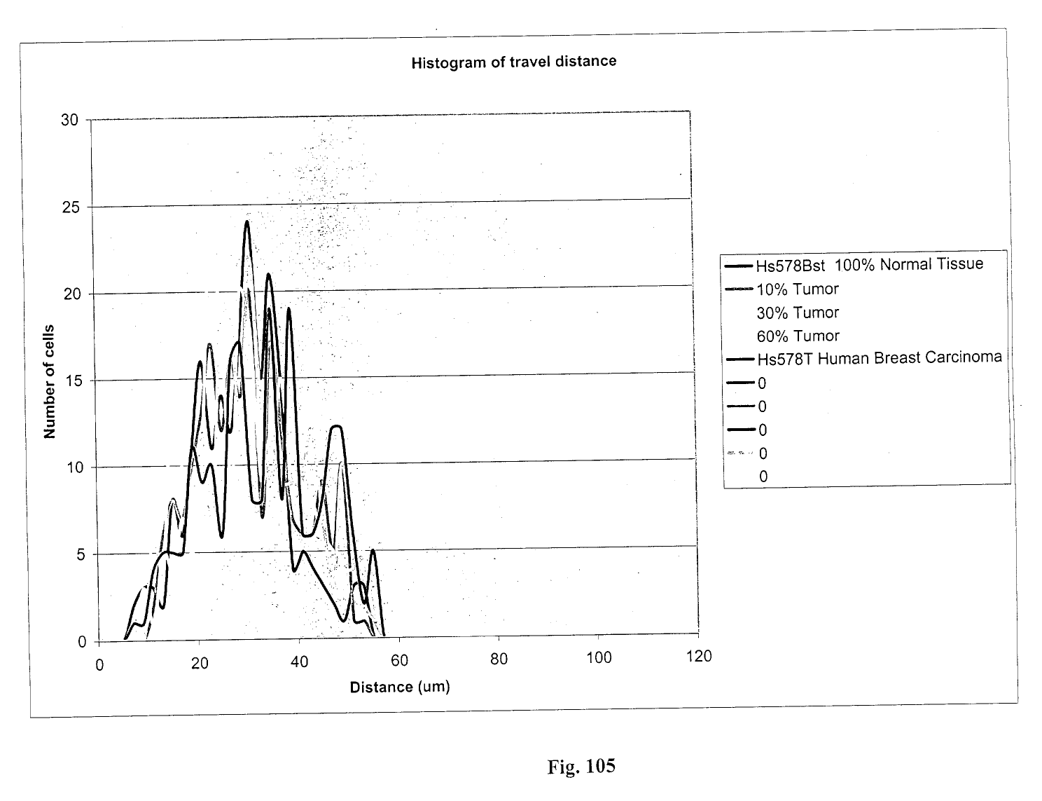

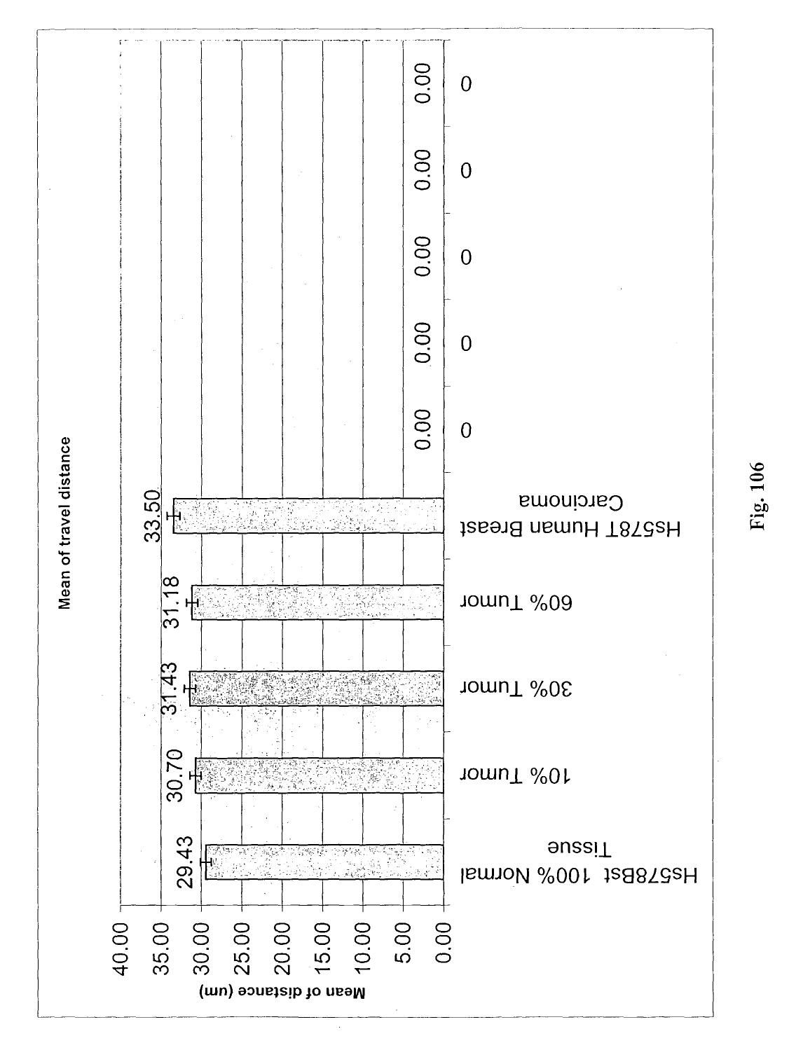

In another experiment, mixed populations of cancerous and non-cancerous breast tissue cells (HS578T and HS578BST) were subject to fast scan analysis. The tests were performed on a sample containing 100% non-cancerous HS578BST cells, a sample containing 10% (by number) of cancerous HS578T cells in mixture of both cancerous and non-cancerous HS578BST breast tissue cells, a sample containing 30% (by number) of cancerous HS578T cells in mixture of both cancerous and non-cancerous HS578BST breast tissue cells, a sample containing 60% (by number) of cancerous HS578T cells in mixture of both cancerous and non-cancerous HS578BST breast tissue cells, and a sample containing 100% cancerous HS578T cells. FIG. 105 illustrates a histogram of the travel distances for the five samples. FIG. 106 illustrates the mean travel distances for each of the five samples. A general trend is seen both FIGS. 105 and 106 wherein samples having increased percentages of cancer cells exhibit larger travel distances.

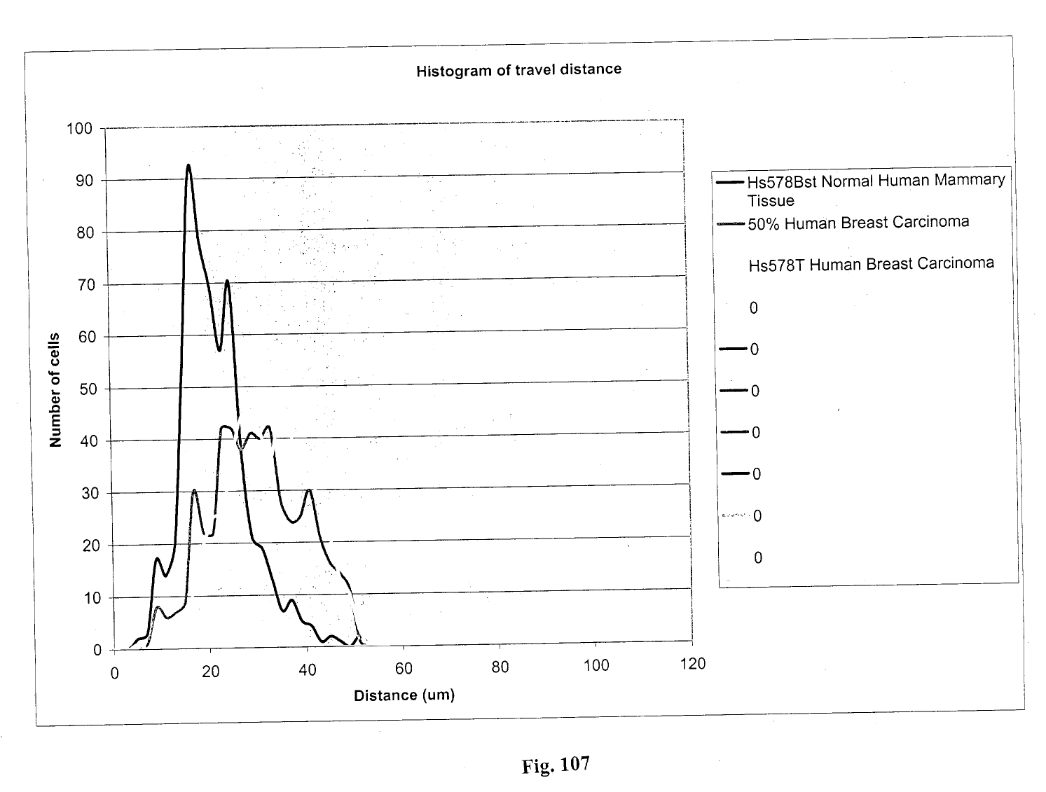

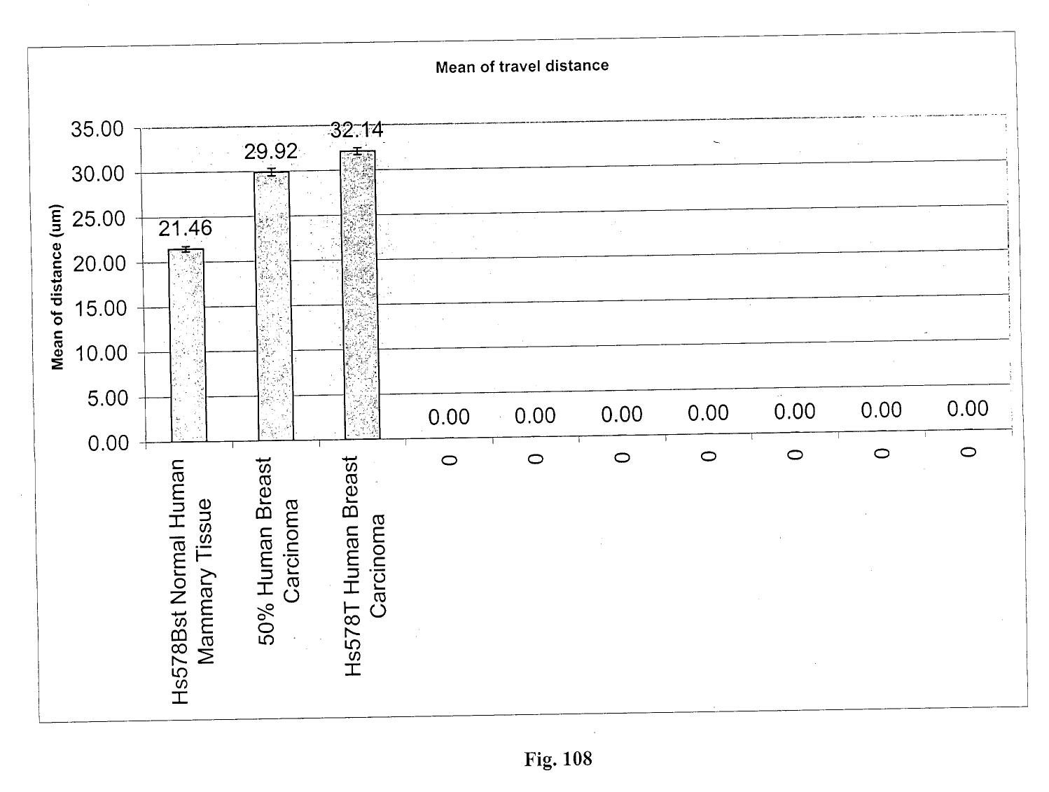

Yet another experiment was performed with three samples, one sample having 100% normal HS578BST cells, another having 50% (by number) of cancerous HS578T cells in a mixture of cancerous and non-cancerous cells, and a sample containing 100% cancerous HS578T cells. A histogram of the travel distances for each cell type is shown in FIG. 107. The mean travel distances of each sample is shown in FIG. 108. Again, a general trend is seen in which travel distance increases as the percentage of cancer cells in the sample increases.

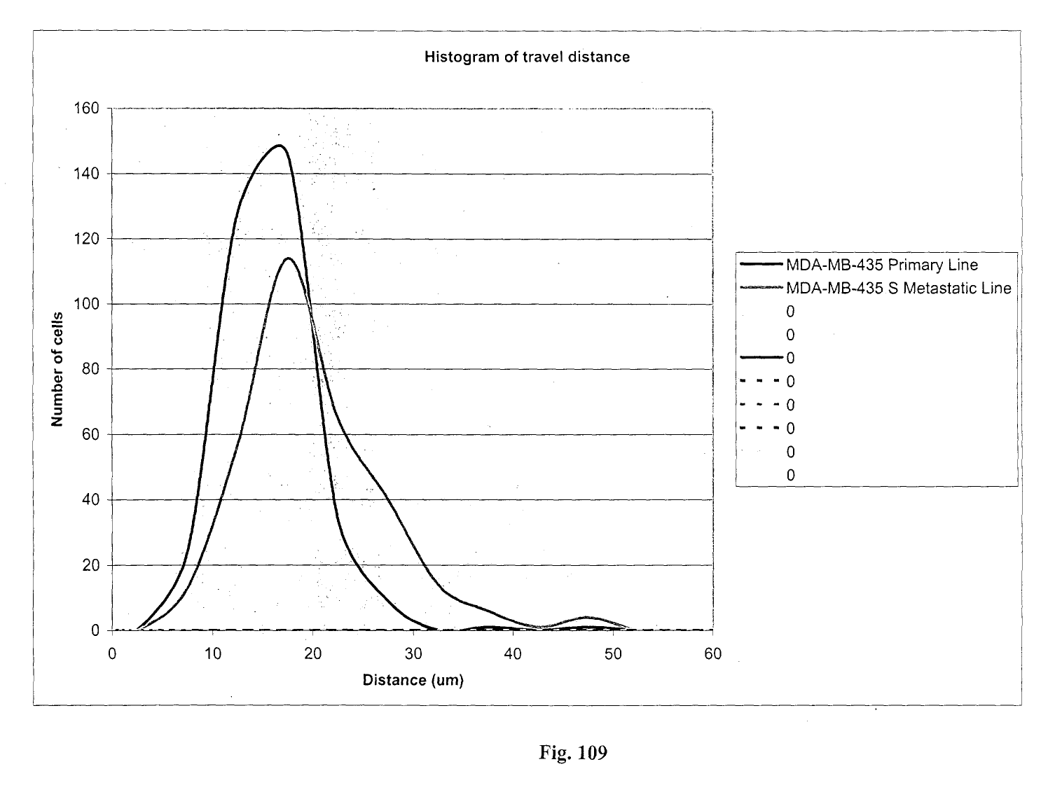

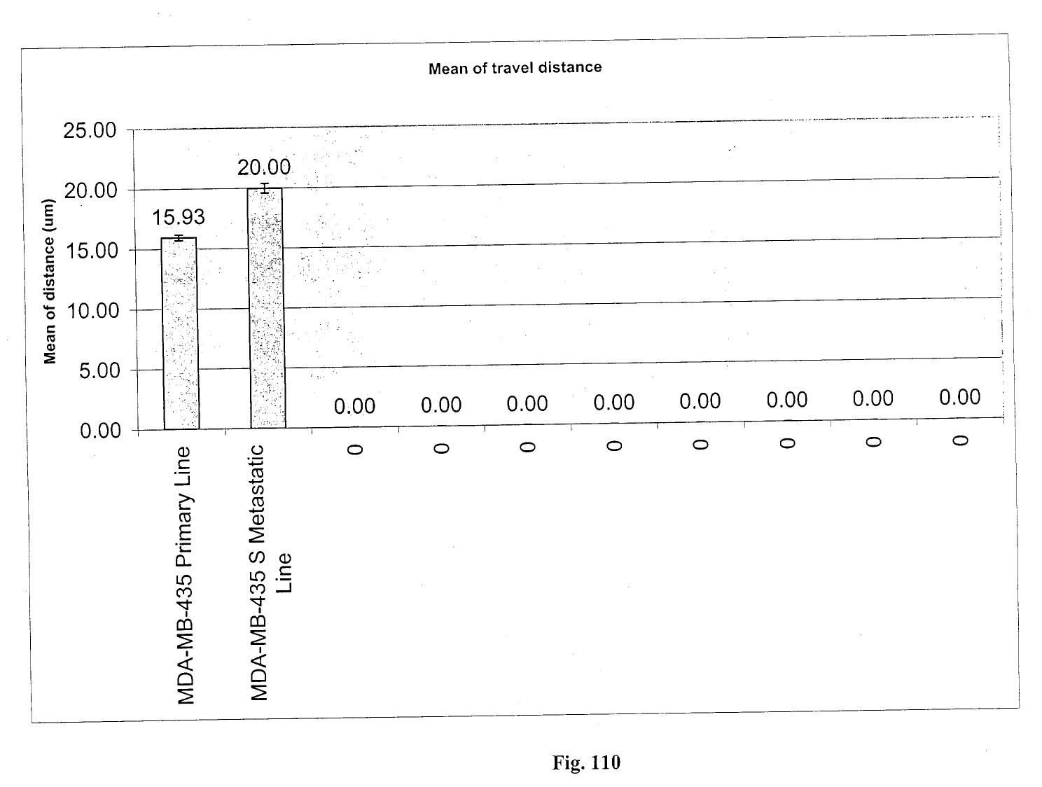

In yet another experiment, two very closely related cancer cells (MDA-MB-435 and MDA-MB-435S) were subject to fast scan analysis. These two cell lines differ slightly in their cellular morphologies. The results of the fast scan test indicates that the 435S line of cells has a slightly larger mean travel distance than the 435 cell line. The histogram of the travel distances for this experiment as well as the mean travel distance is shown in FIGS. 109 and 110.

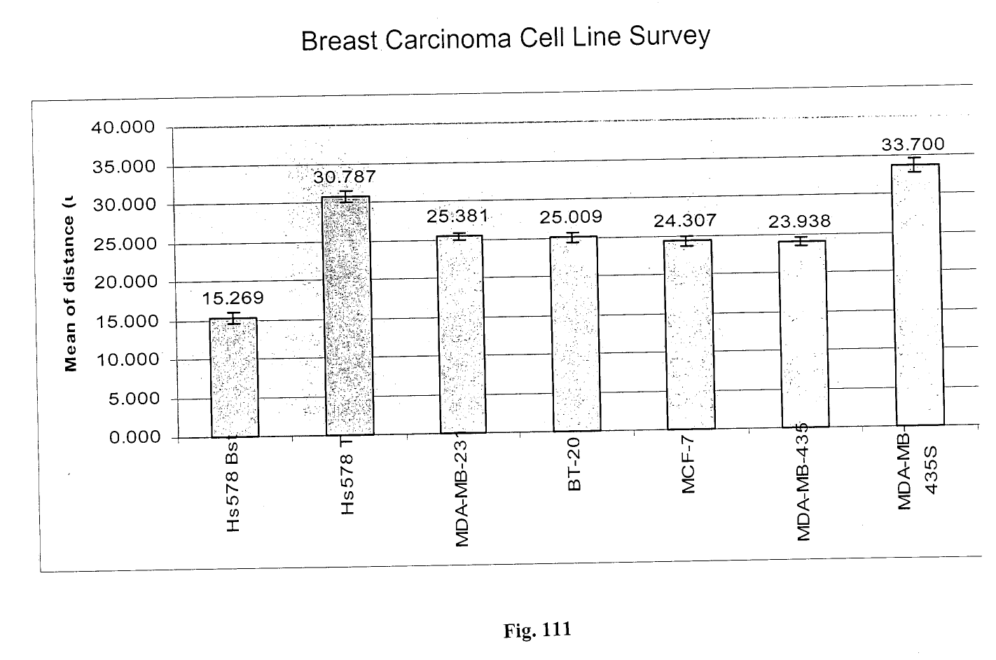

FIG. 111 summarizes the results of additional fast scan testing performed on various breast carcinoma cell lines (HS578T, MDA-ME-231, BT-20, MCF-7, MDA-ME-435, and MDA-MB-435S) as compared to non-cancerous HS578BST cells. As seen in FIG. 111, each of the cancerous cell lines have higher mean travel distance values than the normal HS578BST cells.

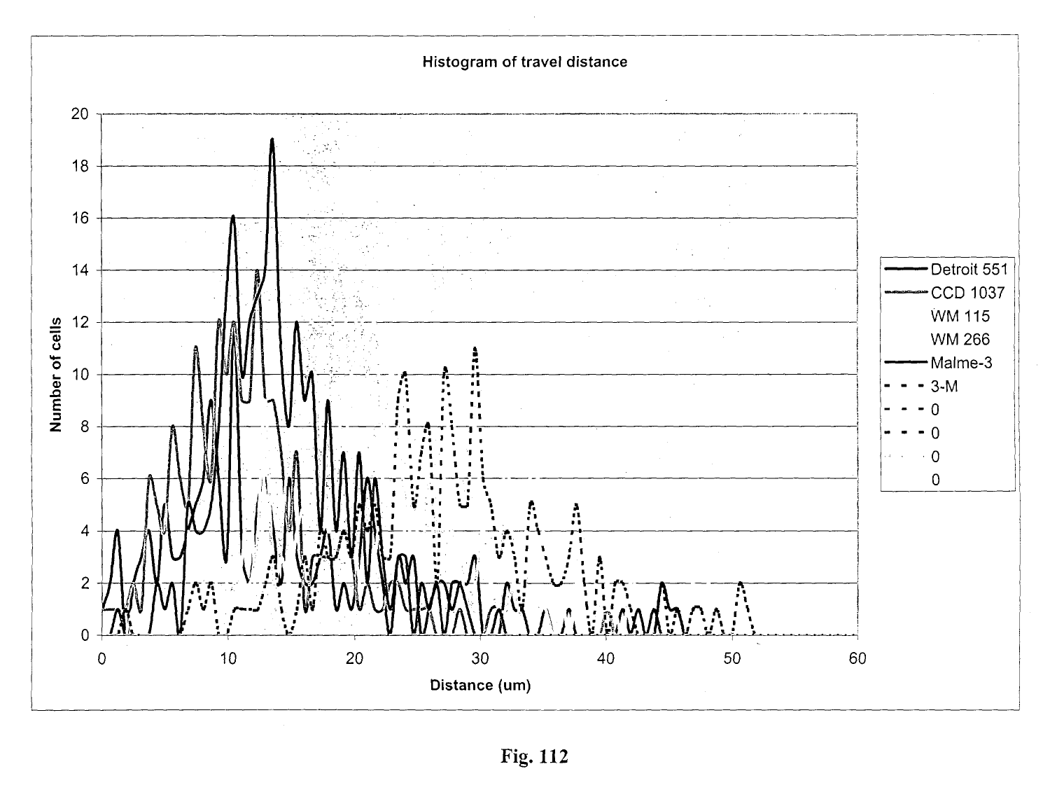

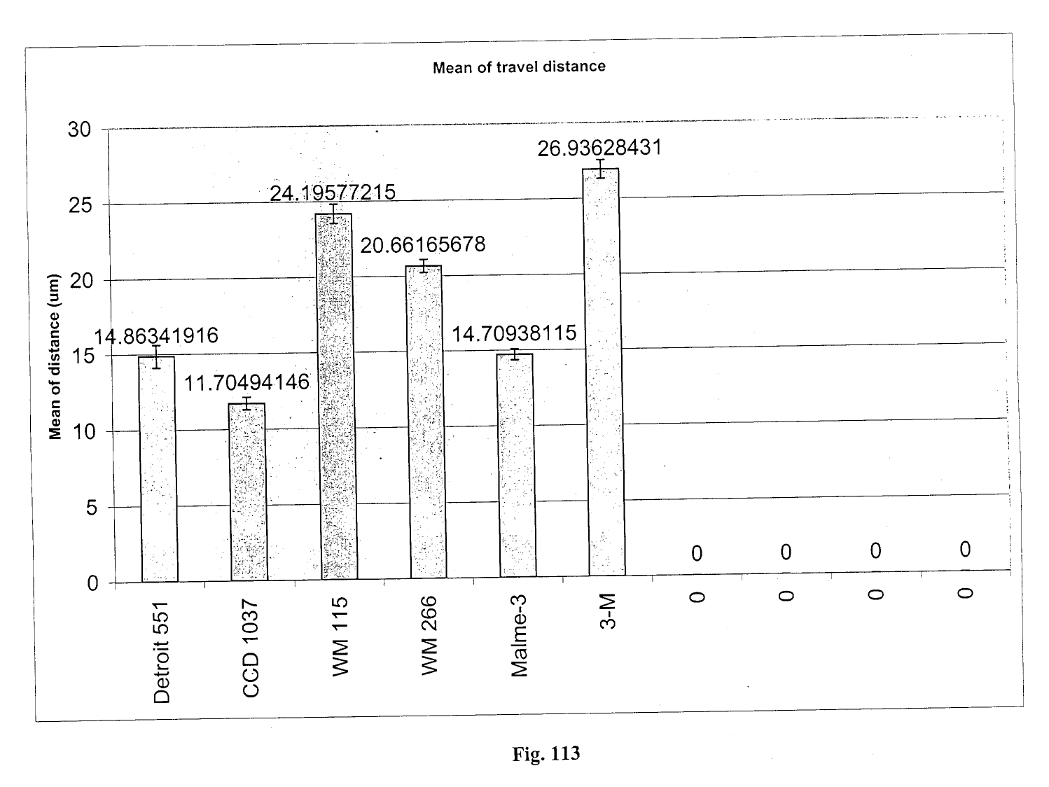

In yet another experiment, a fast scan analysis was performed on six skin cell types. Three of the cell types comprised normal skin cells (Detroit 551, CCD 1037, and Malme-3). The remaining three samples included the WM 266-4 malignant melanoma cell line, the matched WM 115 primary malignant melanoma cell line, and the 3-M malignant melanoma cell line. The 3-M malignant melanoma cells are matched with the Malme-3 (normal) cell line. FIG. 112 illustrates the histogram of the travel distances of the six skin cell types. The mean travel distances for each of the six cell types is shown in FIG. 113. As seen in FIGS. 112 and 113, the normal skin cell lines (Detroit 551, CCD 1037, and Malme-3) had the lowest mean travel distances. The three other malignant melanoma cell lines all had higher mean travel distances. With respect to the matched Malme-3 and 3-M cell lines, the malignant melanoma cell line (3-M) had a significantly higher mean travel distance.

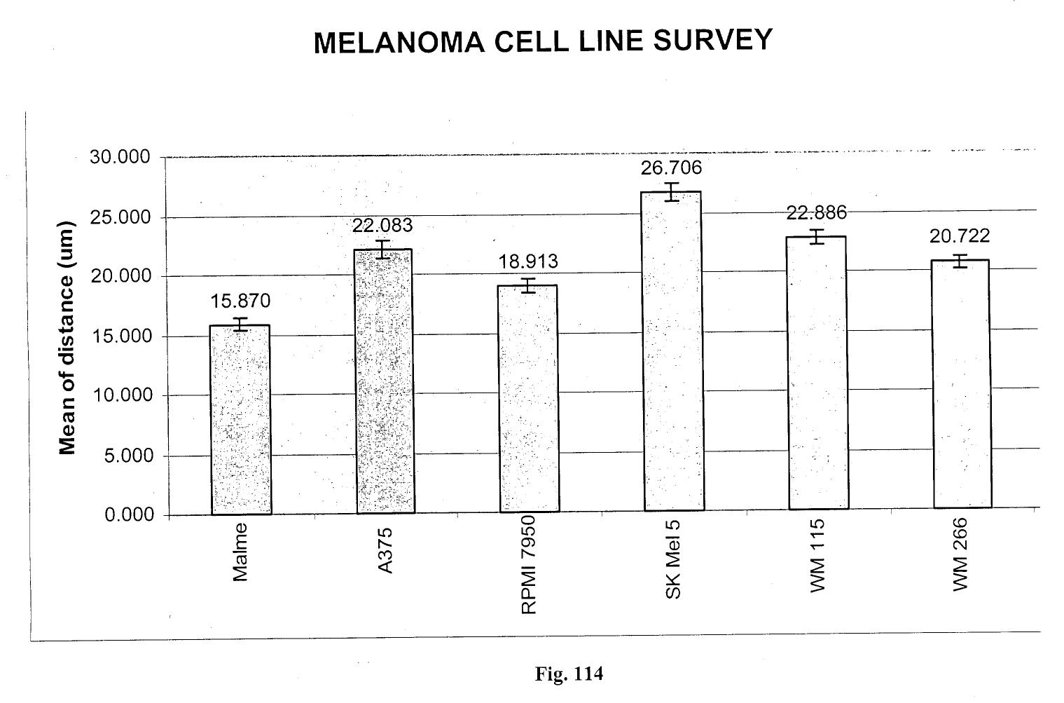

FIG. 114 summarizes the results of additional fast scan testing performed on various malignant melanoma cell lines (A375, RPMI 7950, SKMeI 5, WM 115, WM 266) as compared to non-cancerous Malme cells. As can be seen in FIG. 114, each of the various cancerous cell lines have higher mean travel distance values than the normal Malme cells.

Optophoretic Analysis of Chemically-Mediated and Ligand Mediated T-Cell Activation

Optophoretic analysis was performed on human activated and naive T-cells. Conventional techniques used to distinguish T-cells from other immune cells, such as FACS analysis, RT-PCR, and other technologies, all require the characterization and isolation of an antigen specific to T-cells. Unfortunately, these methods and techniques are labor-intensive and time consuming. Optophoretic analysis techniques, including fast scan analysis, are able to distinguish activated T-cells from naive T-cells. It has been observed that Optophoretic shifts distinguishing active from naive T-cells were generally consistent, large, and dose-dependent. In addition, the results correlated with observed expression markers and secreted cytokines that are associated with T-cell activation.

Because T-cells are critical to the proper functioning of the mammalian immune system, characterization and evaluation of these cells is helpful for use in diagnosing and treatment (i.e., immunotherapy) of major debilitating diseases such as, for example, cancer, autoimmune diseases, graft vs. host disease, and immune deficiency syndromes. The methods and analysis techniques described herein may be used for the evaluation of high affinity T-cells, non-functioning antigen-specific T-cells, as well as immune evaluation of immunotherapeutic vaccines.

Optophoretic analysis was performed on T-cells obtained from normal donor whole blood by negative selection. Red blood cells were lysed from the sample and peripheral blood mononuclear cells (PBMCs) were isolated using density gradient centrifugation. The resulting PBMCs were incubated with an antibody cocktail designed to bind to all cells except T-cells. The T-cells were then collected from elution of a human T-cell enrichment column (obtained from R&D Systems, Inc., 614 McKinley Place N.E., Minneapolis, Minn. 55413, Catalog Number HTCC-5/10/25) which retains non T-cell PBMCs. The enriched T-cell population were then activated using chemical-mediated activation or ligand-mediated activation. The activated T-cells were then subject to Optophoretic analysis and compared with unactivated (naive) T-cells from the same normal donor.

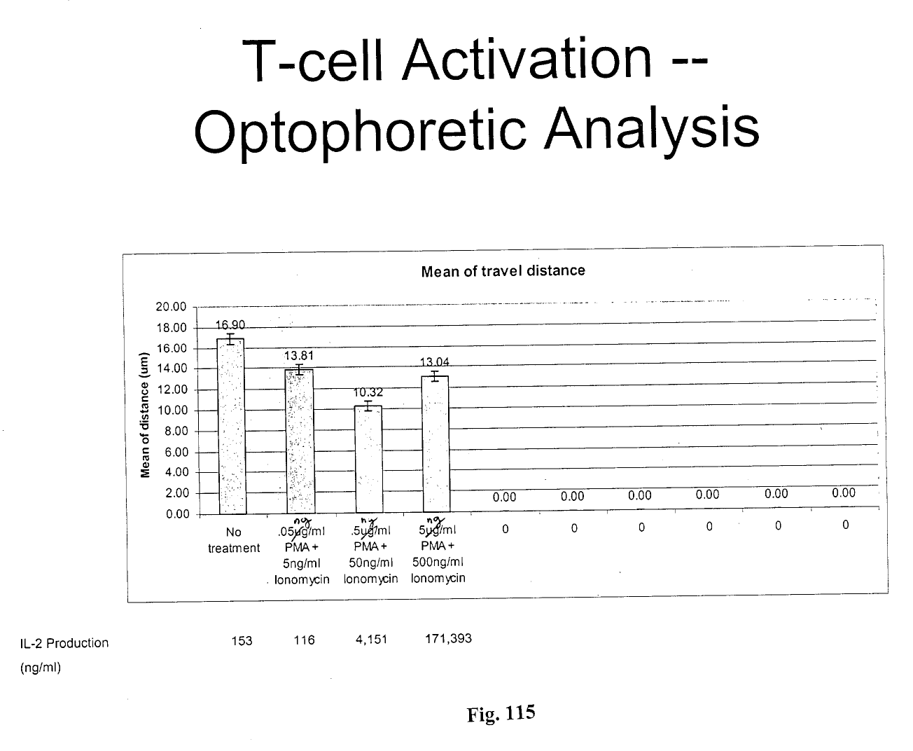

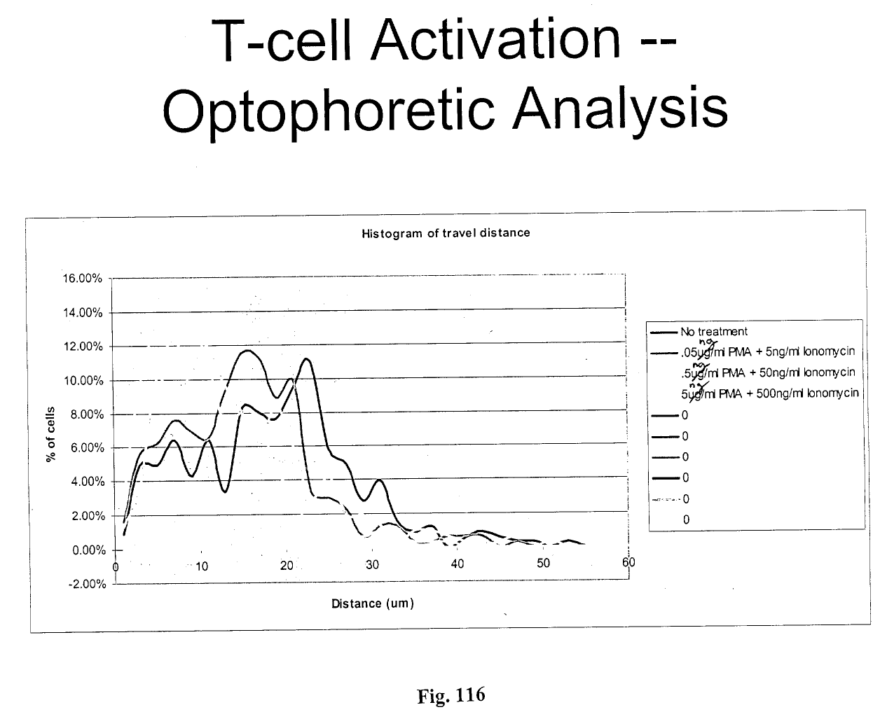

In an first experiment, chemically activated T-cells were subject to Optophoretic analysis using a fast scan analysis. Three groups of T-cells were treated with various levels of phorbol mystirate acetate (PMA) and ionomycin to activate the T-cells. A first group of T-cells was treated with 0.05 ng/ml PMA and 5 ng/ml ionomycin. A second group of T-cells was treated with 0.5 ng/ml PMA and 50 ng/ml ionomycin. A third group of T-cells was treated with 5 ng/ml PMA and 500 ng/ml ionomycin. A fourth control group contained untreated T-cells. The T-cells were subject to Optophoretic analysis on a fast scan instrument after overnight incubation with the PMA and ionomycin. FIG. 115 illustrates the mean travel distances for the four groups of cells described above. FIG. 116 shows the histogram of travel distance.

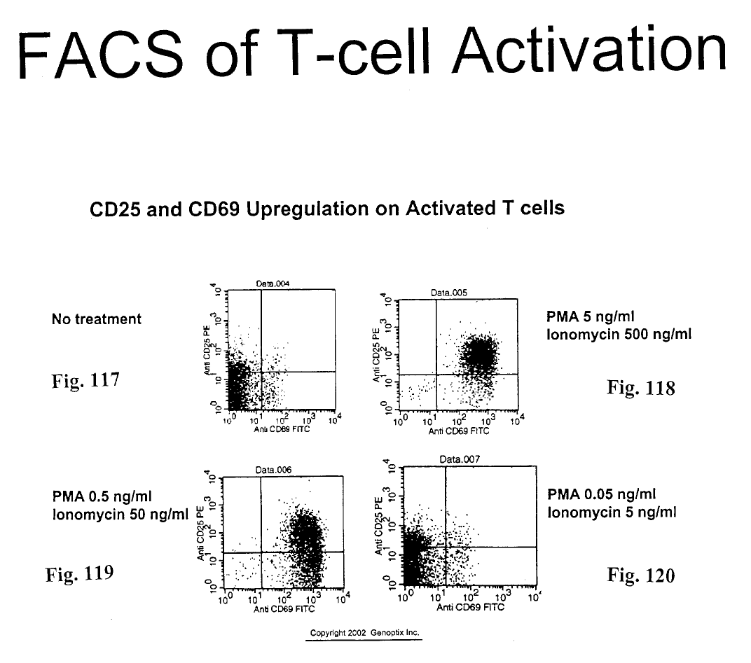

Several different confirmatory tests were performed on the four groups of T-cells to confirm the activation of the treated T-cell groups. In a first confirmation test, the four groups of cells were treated with anti-CD25-PE and anti-CD69-FITC antibodies. The anti-CD25-PE antibody binds to the interleukin 2 (IL-2) receptor on activated T-cells. The anti-CD69-FITC antibody binds an early marker that is known to identify activated T-cells. The labeled T-cells were then subject to FACS analysis and counting. FIGS. 117 through 120 illustrate the FACS results for the four groups of cells. The FACS analysis results confirm that significant activation is seen in the T-cells treated with 0.5 ng/ml PMA and 50 ng/ml ionomycin as well as the T-cells were treated with 5 ng/ml PMA and 500 ng/ml ionomycin. The T-cells treated with the lowest concentrations of PMA and ionomycin (0.05 ng/ml PMA and 5 ng/ml ionomycin) showed a smaller yet statistically significant increase in activation over the control group.

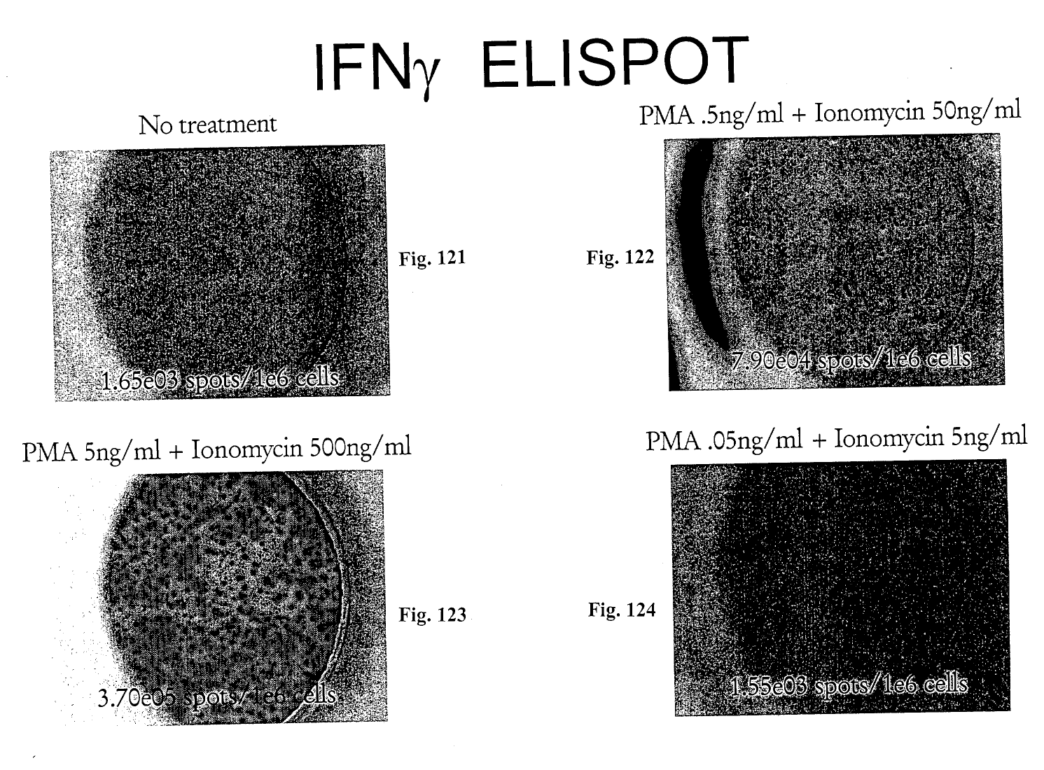

A second confirmatory test was performed using a BD ELISPOT Human IFN-γ kit, available form BD Biosciences, Pharmingen division, to confirm activation of the T-cells. The test is capable of enumerating and characterizing the nature of individual IFN-γ-producing T-cells. FIGS. 121-124 illustrates the results of the BD ELISPOT confirmatory test. As seen in FIGS. 121 through 124, a significant increase in spot count is seen in the T-cells treated with 0.5 ng/ml PMA and 50 ng/ml ionomycin as well as the T-cells were treated with 5 ng/ml PMA and 500 ng/ml ionomycin.

Finally, the results were further confirmed using a Human IL-2 ELISA assay obtained from R&D Systems, Inc. The T-cells were tested after about 24 hours of drug administration. The concentration of T-cells was on the order of 10⁶ cells/ml. The T-cells that were untreated exhibited a production level of IL-2 of 153 pg/ml per 10⁶ cells per 24 hrs. The T-cells that were treated with 0.05 ng/ml PMA and 5 ng/ml ionomycin exhibited a production level of IL-2 of 116. The T-cells that were treated with 0.5 ng/ml PMA and 50 ng/ml ionomycin exhibited a production level of IL-2 of 4,151. The T-cells that were treated with 5 ng/ml PMA and 500 ng/ml ionomycin exhibited a production level of TL-2 of 171,393. The IL-2 production levels of the three samples and the control is shown on the x-axis of FIG. 115.

Tables 19, 20, 21, and 22 reproduced below summarize the results of the T-cell activation tests performed on the four test types, namely, Optophoretically (e.g., fast scan), FACS, Human IFN-γ ELISPOT, and Human IL-2 ELISA.

TABLE 19

| | | 5 ng/ml PMA and | .5 ng/ml PMA | |
|---|---|---|---|---|
| | | 500 ng/ml | and 50 ng/ml | .05 ng/ml PMA and |
| FAST SCAN | No Treatment | ionomycin | ionomycin | 5 ng/ml ionomycin |
| Mean | 16.9 | 13.04 | 10.32 | 13.81 |
| Error of | 0.53 | 0.48 | 0.47 | 0.48 |
| Mean | | | | |
| CV | 56.79% | 64.36% | 80.48% | 60.08% |
| Shift | N/A | −22.9% | −38.9% | −18.3% |
| T-Test | N/A | 9.08E−08 | 2.99E−19 | 1.64E−05 |
| SNR | N/A | 5.33 | 9.14 | 4.26 |

TABLE 20

| | | 5 ng/ml PMA and | .5 ng/ml PMA | .05 ng/ml PMA and |
|---|---|---|---|---|
| | | 500 ng/ml | and 50 ng/ml | 5 ng/ml |
| FACS | No Treatment | ionomycin | ionomycin | ionomycin |
| % Double positive | 1.13 | 89.32 | 65.67 | 2.14 |
| of gated T-cells | | | | |
| % only anti CD69 | 5.02 | 9.79 | 33.39 | 4.04 |
| positive of gated | | | | |
| cells | | | | |
| % only anti CD25 | 16.5 | 0.04 | 0.14 | 24.62 |
| positive gated | | | | |
| cells | | | | |
| Anti CD69 Mean | 47.01 | 668.34 | 771.59 | 43.6 |
| Flourescence | | | | |
| (Histogram) | | | | |
| Anti CD69 Peak | 15 | 716 | 1218 | 21 |
| Channel | | | | |
| (Histogram) | | | | |
| Anti CD25 Mean | 34.52 | 124.06 | 121.19 | 38.91 |
| Flourescence | | | | |
| (Histogram) | | | | |
| Anti CD25 Peak | 8 | 115 | 96 | 16 |
| Channel | | | | |
| (Histogram) | | | | |

TABLE 21

| | | | | .05 |
|---|---|---|---|---|
| Human | | 5 ng/ml PMA | .5 ng/ml PMA | ng/ml PMA |
| IFN-γ | No | and 500 ng/ml | and 50 ng/ml | and 5 ng/ml |
| ELISPOT | Treatment | ionomycin | ionomycin | ionomycin |
| Spots/ | 1.65E+03 | 3.70E+05 | 7.90E+04 | 1550 |
| million | | | | |
| Cells | | | | |

TABLE 22

| Human | | 5 ng/ml PMA | .5 ng/ml PMA | .05 ng/ml PMA |
|---|---|---|---|---|
| IL-2 | No | and 500 ng/ml | and 50 ng/ml | and 5 ng/ml |
| ELISA | Treatment | ionomycin | ionomycin | ionomycin |
| Human | 1.50E−01 | 1.71E+02 | 4.15E+00 | 1.20E−01 |
| IL-2 | | | | |
| production | | | | |
| (ug/ml)/ | | | | |
| 1e6 | | | | |
| cells/ml | | | | |

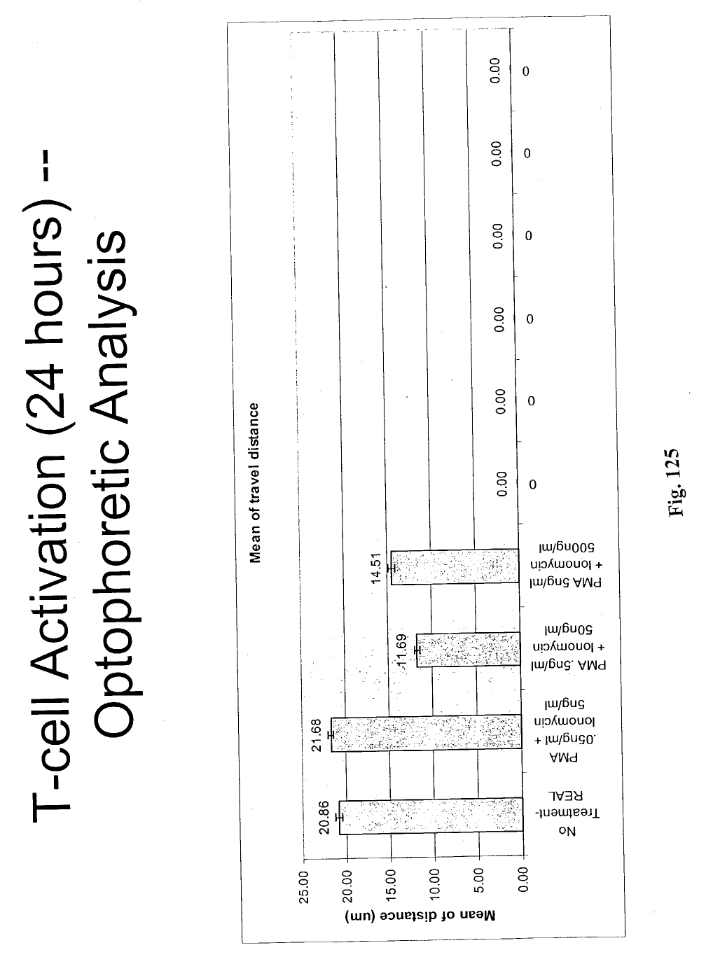

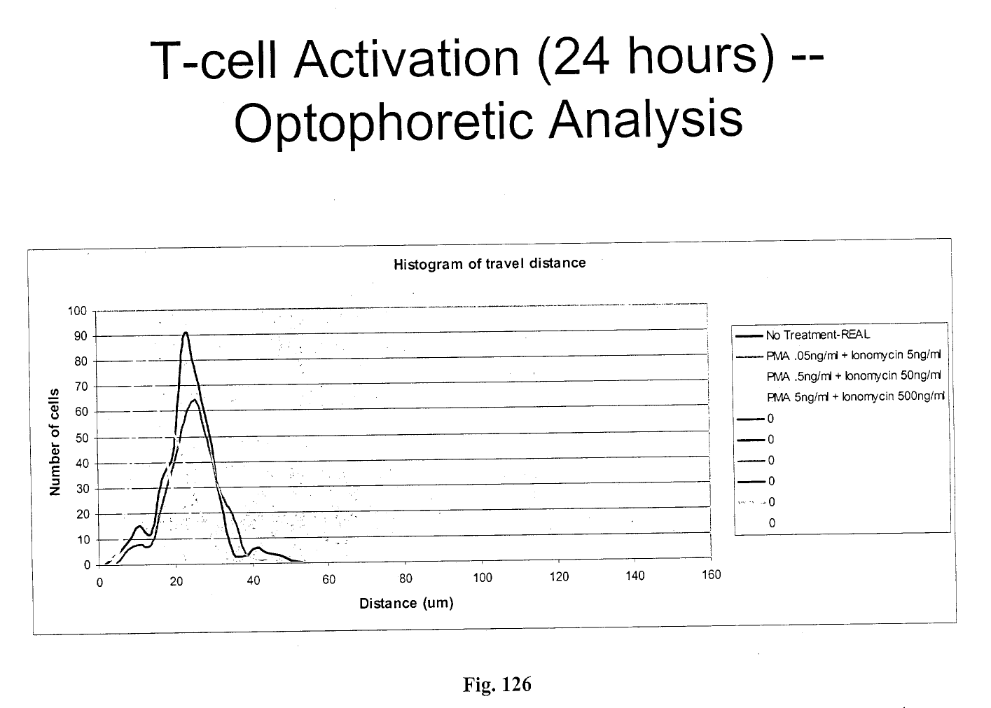

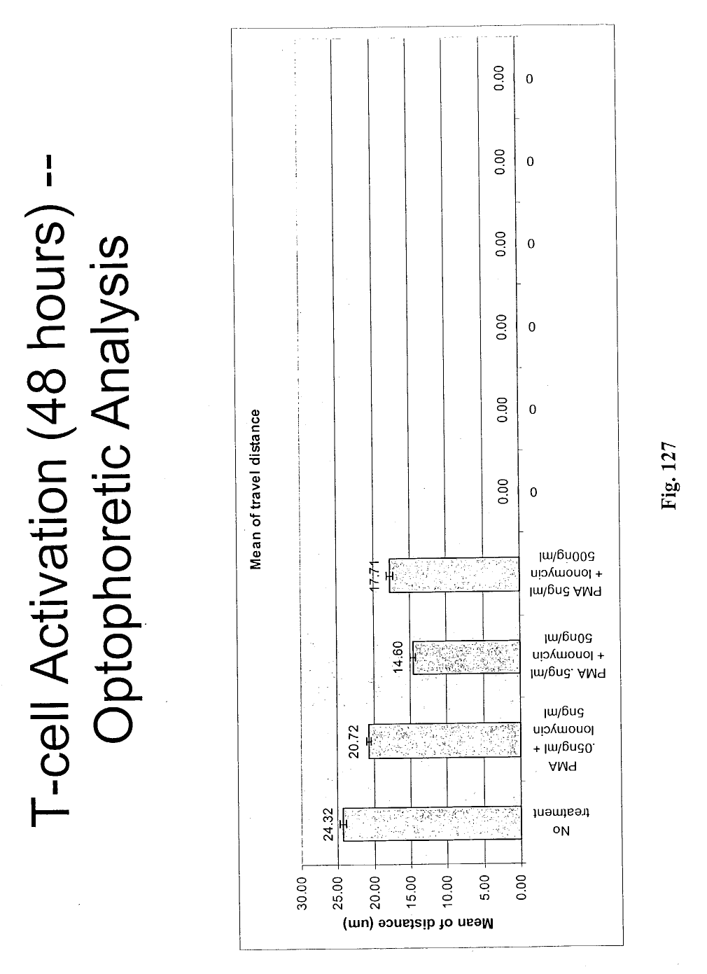

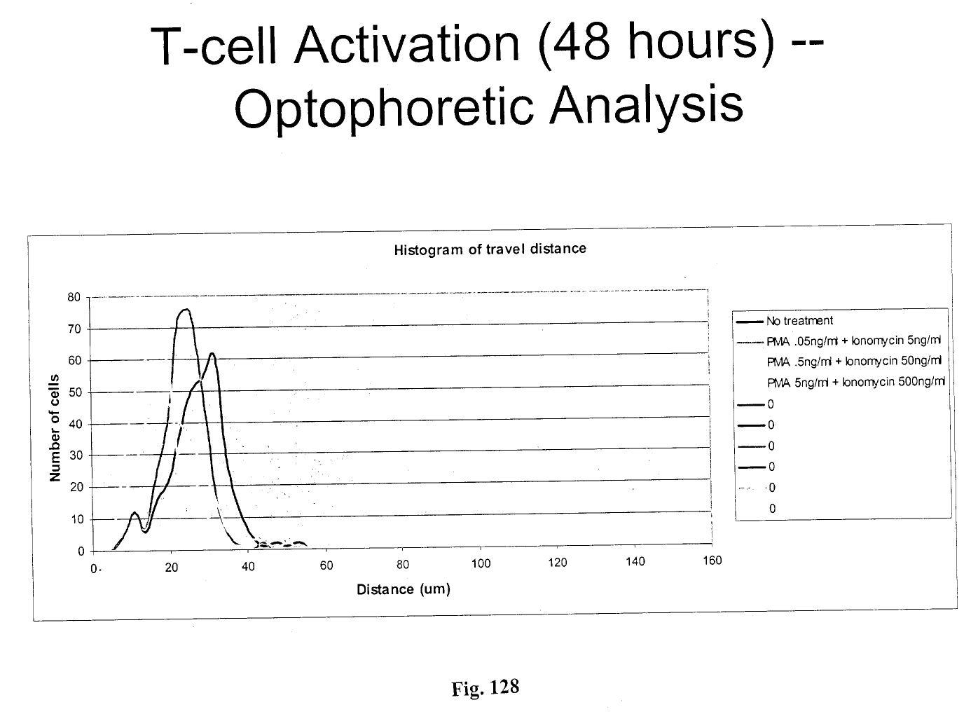

In another experiment, the activation of T-cells with the mixture of PMA and ionomycin was measured at 24 hours and 48 hours after incubation. Three groups of T-cells were treated with different levels of phorbol myristate acetate (PMA) and ionomycin to activate the T-cells. The different combinations of the mixtures was identical to that present in the prior experiment (i.e., 0.05 ng/ml PMA and 5 ng/ml ionomycin; 0.5 ng/ml PMA and 50 ng/ml ionomycin; 5 ng/ml PMA and 500 ng/ml ionomycin). A control group of T-cells were untreated. The T-cells were subject to Optophoretic analysis on a fast scan instrument after 24 hours and 48 hours incubation with the PMA and ionomycin. FIG. 125 illustrates the mean travel distances for the four groups of cells at 24 hours after the start of incubation. FIG. 126 illustrates a histogram of the travel distances of the three treated groups plus the control. FIG. 127 illustrates the mean travel distances for the four groups of cells at 48 hours after the start of incubation. FIG. 128 illustrates a histogram of the travel distances of the three treated groups plus the control.

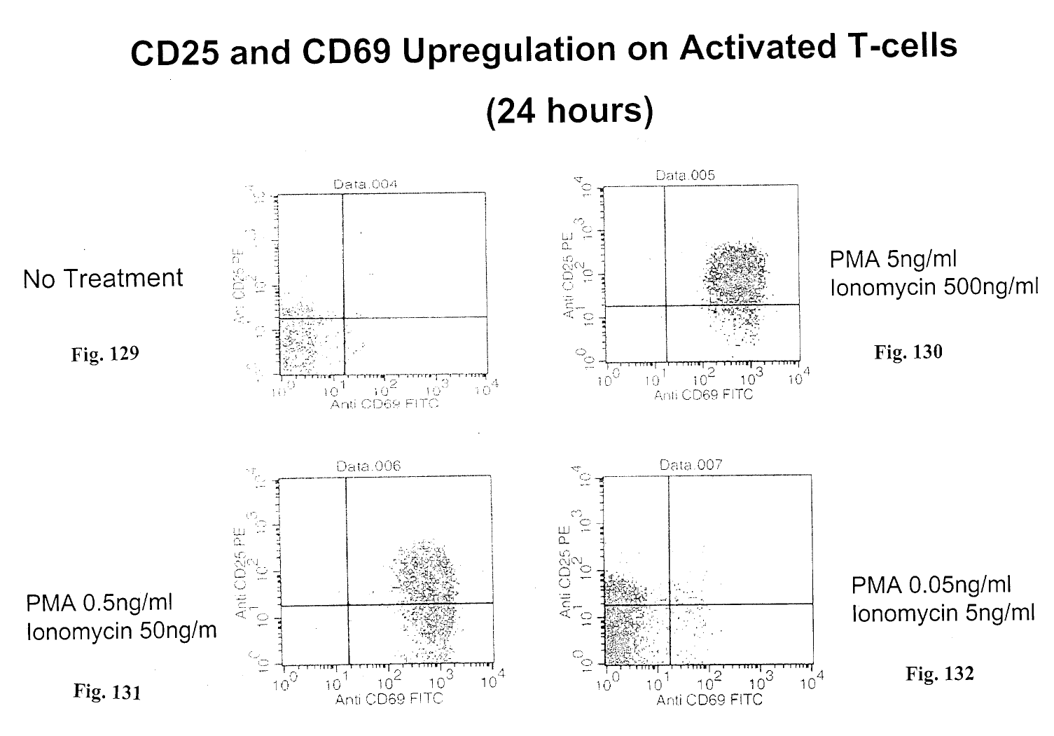

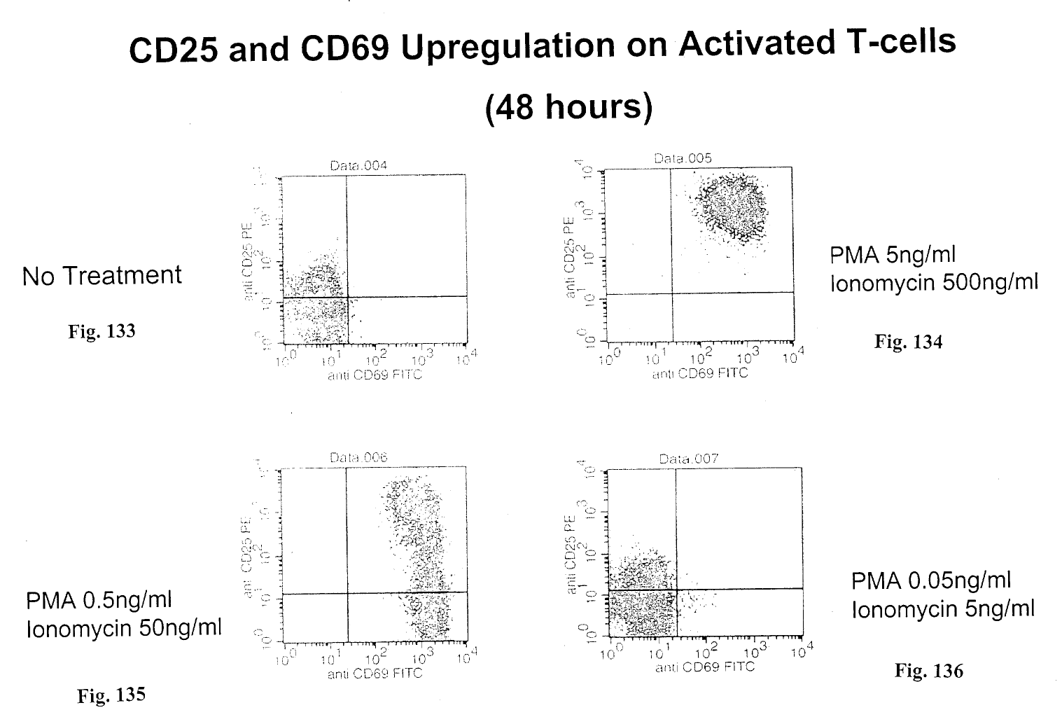

At both 24 and 48 hours post incubation, FACS analysis was performed on all the T-cell groups as a confirmatory test. FIGS. 129-132 show the results of the FACS analysis performed after 24 hours of incubation with the PMA and ionomycin. As can be seen in FIGS. 129-132, a significant increase of cells in the positive gate is seen in the T-cells treated with 0.5 ng/ml PMA and 50 ng/ml ionomycin as well as the T-cells were treated with 5 ng/ml PMA and 500 ng/ml ionomycin. FIGS. 133-136 show the results of the FACS analysis performed after 48 hours of incubation with the PMA and ionomycin. These results, which are similar to the results obtained after 24 hours of incubation, show a significant increase of cells in the positive gate is seen in the T-cells treated with 0.5 ng/ml PMA and 50 ng/ml ionomycin as well as the T-cells were treated with 5 ng/ml PMA and 500 ng/ml ionomycin.

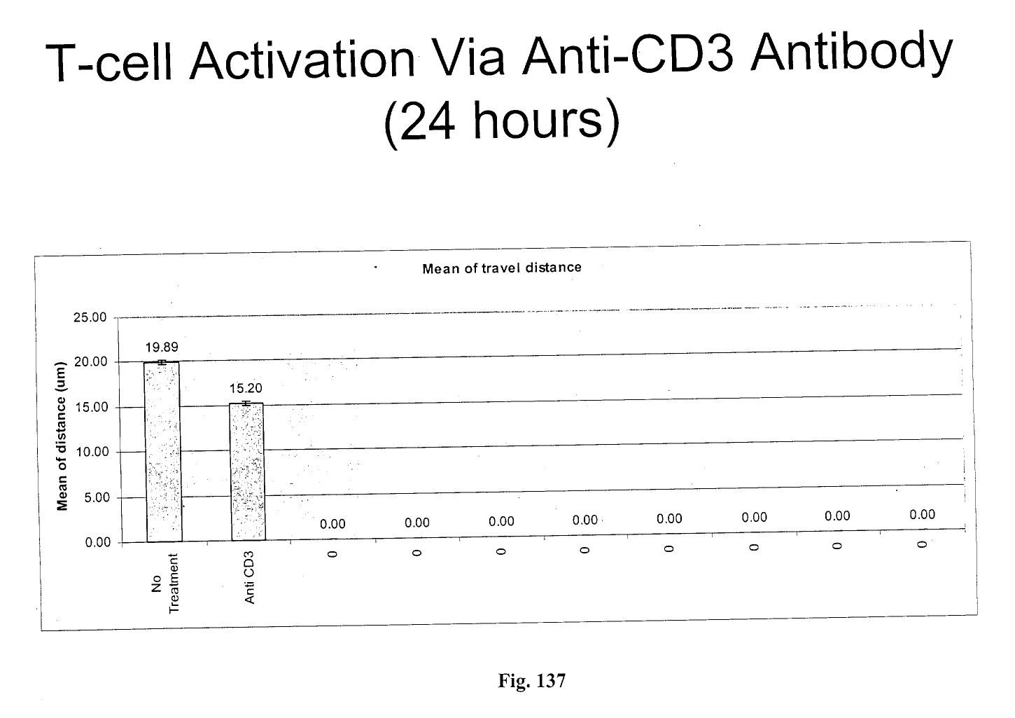

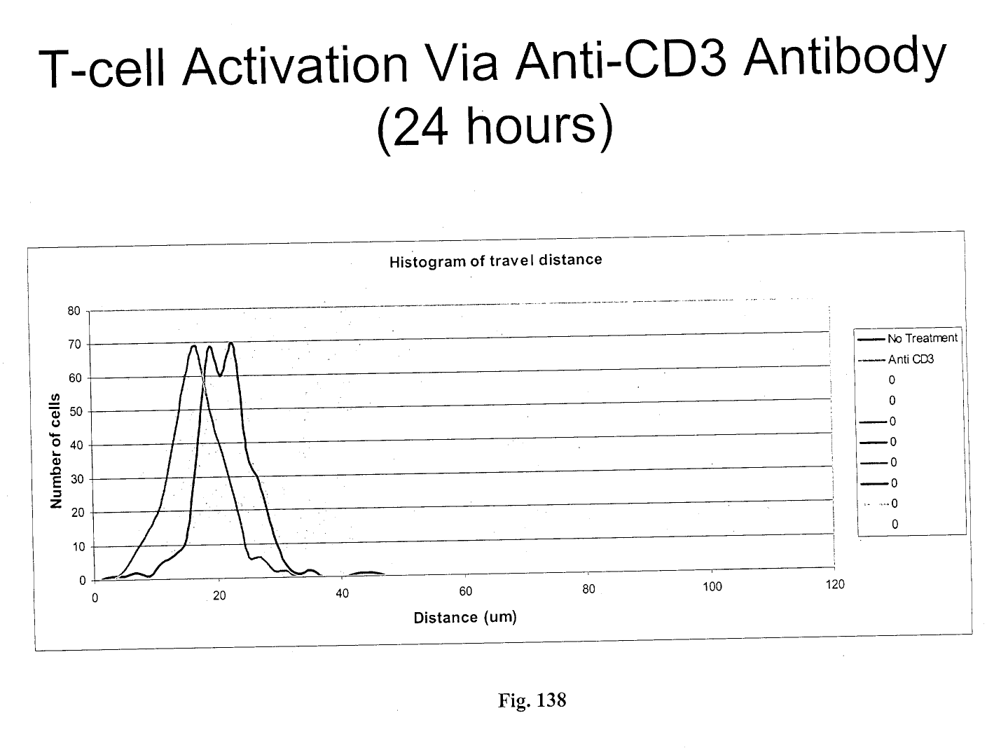

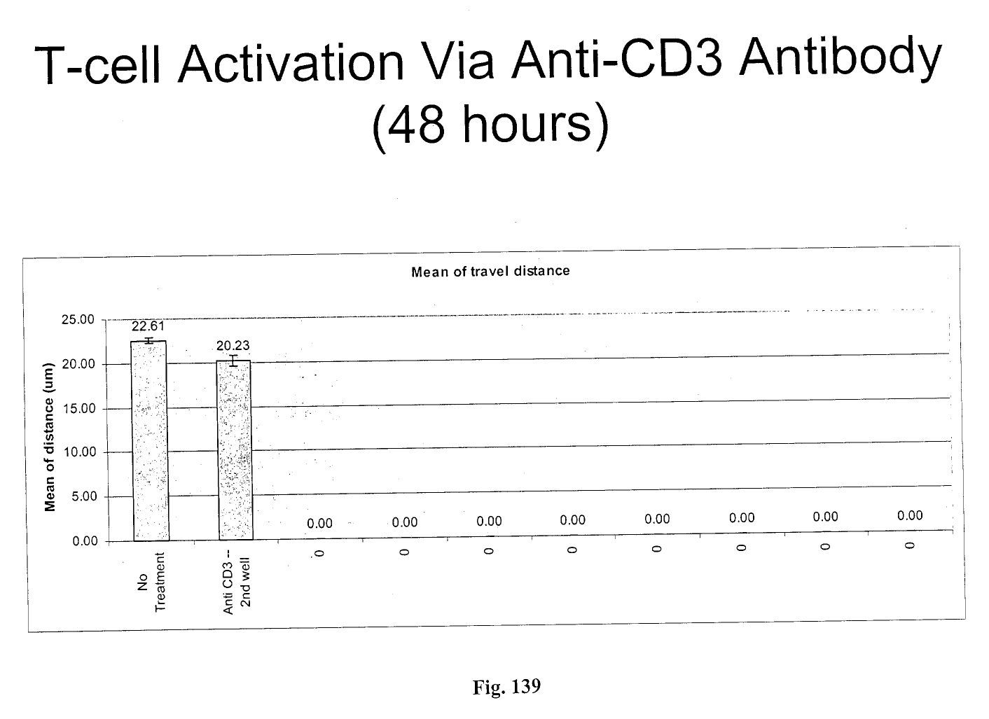

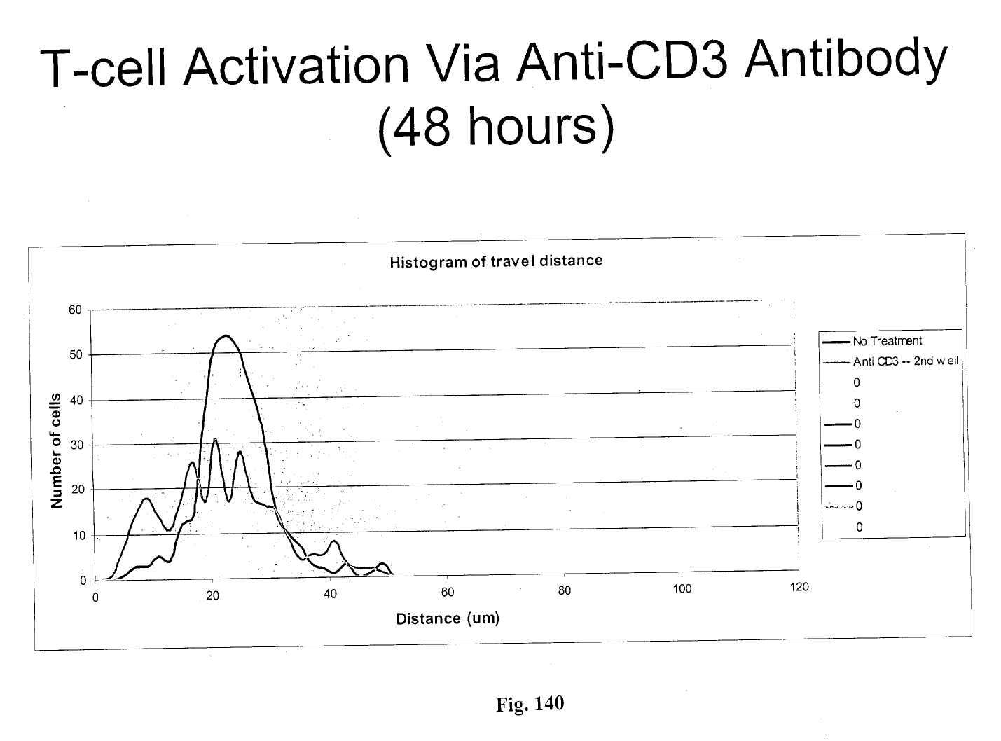

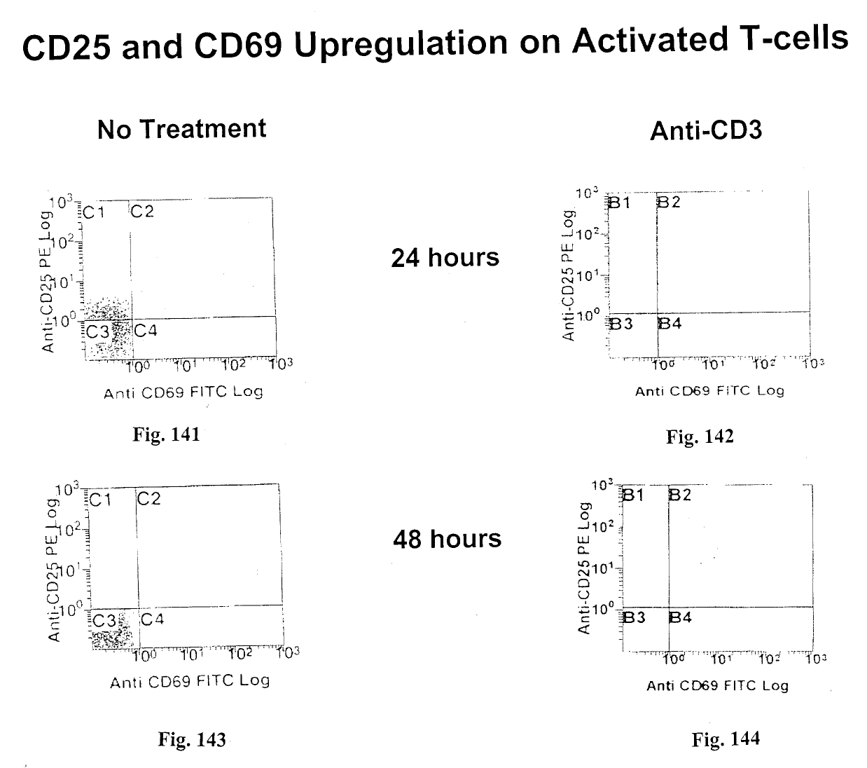

In another experiment, ligand-mediated activation of T-cells was analyzed Optophoretically. In this experiment, T-cells were obtained in the same manner as is described above with respect to the PMA and ionomycin experiments. Unlike the prior experiments, the T-cells were are incubated with an anti-CD3 antibody. The T-cells were subject to Optophoretic analysis on a fast scan instrument after 24 and 48 hour incubation periods with the anti-CD3 antibody. FIG. 137 illustrates the mean travel distances for the treated and non-treated T-cells described above after 24 hours. FIG. 138 shows the histogram of travel distances after 24 hours. FIG. 139 illustrates the mean travel distances for the treated and non-treated T-cells described above after 48 hours. FIG. 140 shows the histogram of travel distances after 48 hours. The results were confirmed with FACS analysis at both 24 and 48 hours after application of the anti-CD3 antibody (FIGS. 141-144). As seen in FIGS. 141-144 significant activity is seen in the FACS results after 24 and 48 hours of treatment with the antibody as compared to the untreated cells.

Early Detection of Cellular Differentiation Using Optophoresis

Currently, in vitro cellular differentiation assays are becoming widely used for drug discovery efforts for identifying compounds having anti-cancer and anti-obesity properties. These same assays are also used in stem cell research and tissue regeneration applications. These current cellular differentiation assays, however, are labor and time intensive. Moreover, these assay methods rely on labels or secondary reagents which, among other things, increases the cost and complexity of the assays. Typically, current assays rely on the expression of known markers indicative of cellular differentiation. The markers, however, may not be present in sufficient detectable levels until well after the onset of cellular differentiation. It has been discovered that Optophoretic techniques may be used in the early detection of cellular differentiation without the use of labels or secondary reagents.

HL-60 is a promyelocytic leukemia cell line that retains the capacity to undergo terminal differentiation and serves as a model system to study myeloid pathways of cellular differentiation. It is known that treatment of HL-60 cells with phorbol 12-myristate 13-acetate (PMA) causes the cells to differentiate into monocytes/macrophages. In addition, it is also known that treatment of HL-60 cells with dimethylsulfoxide (DMSO) causes these same cells to undergo granulocytic differentiation.

Initial tests were performed using HL-60 cells treated with PMA. HL-60 cells were seeded at about 1-3×10⁶ cells per well in six well plates. The cells were treated with 200 ng/ml PMA while the control cells were treated with ethanol (EtOH) vehicle for four hours at 37° C. After incubation for four hours the treated cells were washed with PBS and re-fed with growth media. The treated HL-60 cells were then allowed to differentiate for 72 hours in normal growth media. After the 72 hour period, the untreated control cells were collected by centrifugation, washed with PBS and pelleted for resuspension at 1.5×10⁶ cells/ml in PBS/1% BSA. Treated cells were trypsinized and collected by centrifugation then washed in PBS and pelleted for resuspension at 1×10⁶ cells/ml in PBS/1% BSA. The cells were the subjected to Optophoretic analysis by measuring the escape velocities of the treated and control cells. Table 23 shown below illustrates the measured escape velocities of the undifferentiated and differentiated cells at 72 hours post treatment.

TABLE 23

| | HL-60 Undifferentiated at | HL-60 + PMA at 72 hours |
|---|---|---|
| | 72 hours (Control) | (differentiated) |
| Cell No. | Escape Velocity (μm/sec) | Escape Velocity (μm/sec) |
| 1 | 11.0 | 13.0 |
| 2 | 11.5 | 15.5 |
| 3 | 11.0 | 11.0 |
| 4 | 11.5 | 14.0 |
| 5 | 12.0 | 14.0 |
| 6 | 12.5 | 12.5 |
| 7 | 12.0 | 14.0 |
| 8 | 10.5 | 16.5 |
| 9 | 11.0 | 14.5 |
| 10 | 12.0 | 13.0 |
| 11 | 12.5 | 14.5 |
| 12 | 13.0 | 12.5 |
| 13 | 12.0 | 13.5 |
| 14 | 11.5 | 15.5 |
| 15 | 13.0 | 15.0 |
| 16 | 12.5 | 14.5 |
| 17 | 12.0 | 12.5 |
| 18 | 11.0 | 15.0 |
| 19 | 12.5 | 16.0 |
| 20 | 12.0 | 13.5 |
| Average | 11.9 | 14.0 |

As can be seen from the data in Table 23, the HL-60 cells that were treated with PMA exhibited, on average, an increase of about 2 μm/sec in measured escape velocity as compared to the control cells treated with the EtOH vehicle.

Fast scan analysis was also performed on HL-60 cells that were treated with PMA and DMSO. A time course evaluation of PMA treated HL-60 cells was performed with fast scan data taken at 16 hours, 24 hours, 40 hours, and 72 hours post-treatment with 400 ng/ml PMA. A control sample of HL-60 cells was also tested using EtOH as the vehicle for purposes of comparing the differentiated cells with the undifferentiated HL-60 cells. The PMA-treated and control cells were harvested and trypsinized. The samples were then resuspended in PBS/1% BSA. A set of samples was also obtained and left as pellets at 4° C. for subsequent FACS analysis.

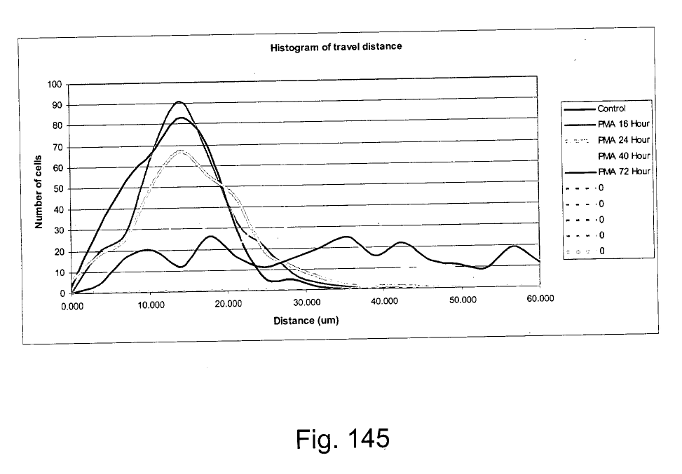

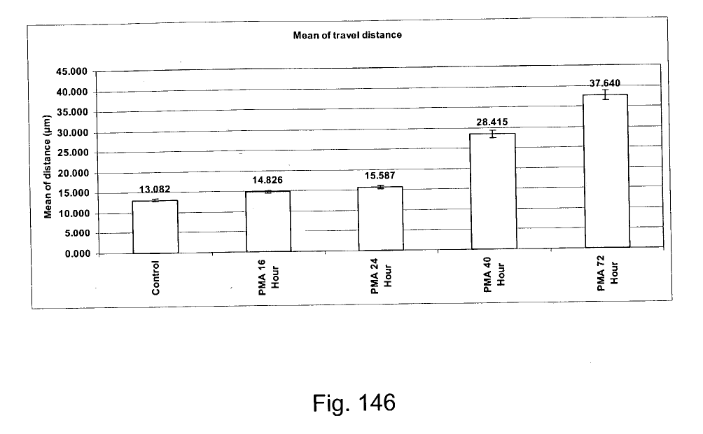

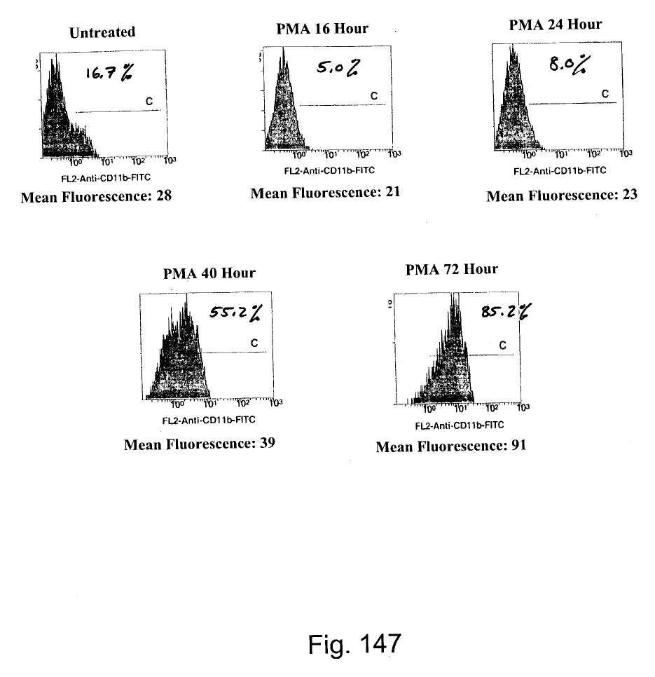

FIG. 145 illustrates a histogram of the travel distance of the control cells as well as the PMA-treated cells at 16 hours, 24 hours, 40 hours, and 72 hours post-treatment. FIG. 146 illustrates the mean travel distances of the control cells as well as the PMA-treated cells at 16 hours, 24 hours, 40 hours, and 72 hours post-treatment. FIG. 147 illustrates the FACS CD11b expression profile in PMA-treated HL-60 cells as well as the control. CD11b is a known marker of cellular differentiation and is expressed on the surface of differentiating cells. As seen in FIGS. 145-146, Optophoretic analysis detected cellular differentiation into monocytes/macrophages at least as early as 16 hours after treatment with PMA. In contrast, using conventional FACS analysis, which measures expression levels of CD11b, a noticeable change in CD11b expression levels was not observed until about 40 hours after treatment with PMA (FIG. 147). The data illustrate that Optophoretic analysis can be used as an assay for the detection and quantification of cellular differentiation. Moreover, Optophoretic analysis is able to provide for an earlier detection of cellular differentiation as compared to conventional techniques that rely on the labeling of expressed markers such as CD11b.

A time course evaluation of DMSO treated HL-60 cells was performed with fast scan data taken at 16 hours, 24 hours, 40 hours, and 70 hours post-treatment with 1% DMSO. A control sample of HL-60 cells was also tested for purposes of comparing the differentiated cells with the undifferentiated HL-60 cells. The DMSO-treated and control cells were then harvested and resuspended in PBS/1% BSA. A set of samples was also obtained and left as pellets at 4° C. for subsequent FACS analysis.

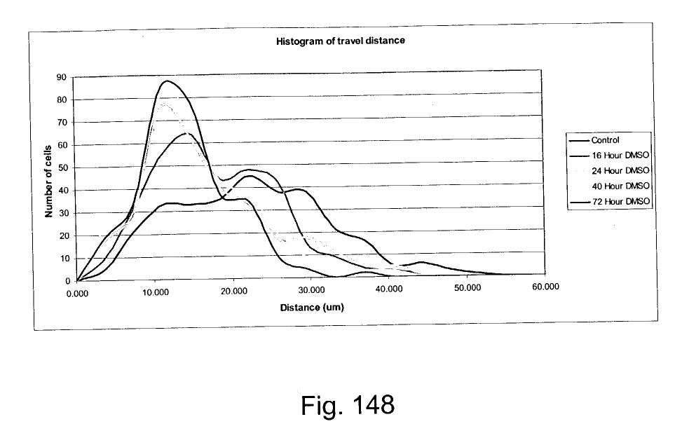

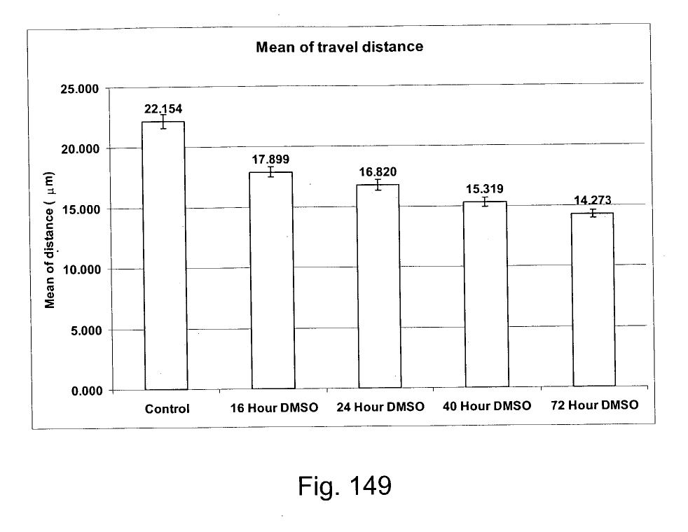

FIG. 148 illustrates a histogram of the travel distance of the control cells as well as the DMSO-treated cells at 16 hours, 24 hours, 40 hours, and 72 hours post-treatment. FIG. 149 illustrates the mean travel distances of the control cells as well as the DMSO-treated cells at 16 hours, 24 hours, 40 hours, and 72 hours post-treatment. FIG. 150 illustrates the FACS CD11b expression profile in DMSO-treated HL-60 cells as well as the control the 16 hour, 24 hour, 40 hour, and 72 hour time points. As seen in FIGS. 148 and 149, at least as early as 16 hours after treatment with DMSO, a noticeable decrease the mean travel distance of the HL-60 cells was detected. The mean travel distance progressively decreased as time elapsed (see FIG. 149). In contrast, conventional FACS analysis of CD11b expression did not detect a change in CD11b expression levels until 40 hours after treatment with DMSO.

The data illustrate that Optophoretic analysis can be used as an assay for the detection and quantification of cellular differentiation. Moreover, Optophoretic analysis is able to provide for an earlier detection of cellular differentiation as compared to conventional techniques that rely on the labeling of expressed markers such as CD11b. Moreover, Optophoretic analysis provides a more sensitive and reproducible manner of assaying cellular programming events. Finally, the Optophoretic analysis method is also able to delineate cells along a particular lineage pathway (i.e., granulocytic vs. monocytic).

Optophoretic Detection of Adipogenesis

Adipocytes are fat cells and play critical roles in energy metabolism and homeostasis. Moreover, there is a growing and increasingly accepted body of evidence that supports the hypothesis that adipose tissue contributes to the pathogenesis of obesity, cardiovascular disease, diabetes, and hypertension. Current research on adipogenesis has been greatly facilitated by the establishment of immortalized adipoblasts or preadipocytes that readily differentiate into adipocytes under particular conditions. Cell lines such as 3T3-L1 and 3T3-F442A undergo differentiation in vitro in six to eight days through a standardized induction regimen that includes cAMP, insulin, and glucocorticoids. More recently, cultured primary mesenchymal stem cells have been used to study adipogenesis, however, the complexity of these cell systems has hindered their extensive use.

Current methodologies for the detection of adipogenesis include lipid stains and fluorescent probes. FIGS. 151(a) and 151(b) illustrate uninduced and 8 day induced adipocytes stained with oil red. FIGS. 152(a) and 152(b) illustrate uninduced and 8 day induced adipocytes stained with BODIPY 505/515 fluorophore (4,4-difluoro-4-bora-3a, 4a-diaza-s-indacene having an absorption maxima at 505 nm (in methanol) and emission maxima at 515 nm).

Optophoretic analysis has been performed on 3T3-L1 mouse cells using fast scan analysis to monitor and detect adipogenesis. 3T3-L1 cells were seeded at about 2×10⁵ cells per well in six well plates (1×10⁴ for 96 well plates for BODIPY 505/515 assay) in 20% calf serum/DMEM. At 100% confluence, the cells were treated with MDI induction media (0.5 mM IBMX, 1 μM dexamethasone, 5 μg/ml human insulin in 10% FBS/DMEM) for the duration of three days, then removed and subsequently replaced with insulin media (10% FBS/DMEM supplemented with 5 μg/ml human insulin) for one day and then every two days thereafter for the duration of the regimen. The uninduced control cells were given 10% FBS/DMEM media only. At eight days post-induction, all of the cells were harvested using a mixture of Versene-EDTA and an anti-aggregation agent. A time course evaluation of the induced 3T3-L1 cells was performed with fast scan data taken at 2, 4, 6, and 8 days post-induction. An uninduced control sample of 3T3-L1 cells was also tested for purposes of comparing the differentiated cells with the undifferentiated 3T3-L1 cells.

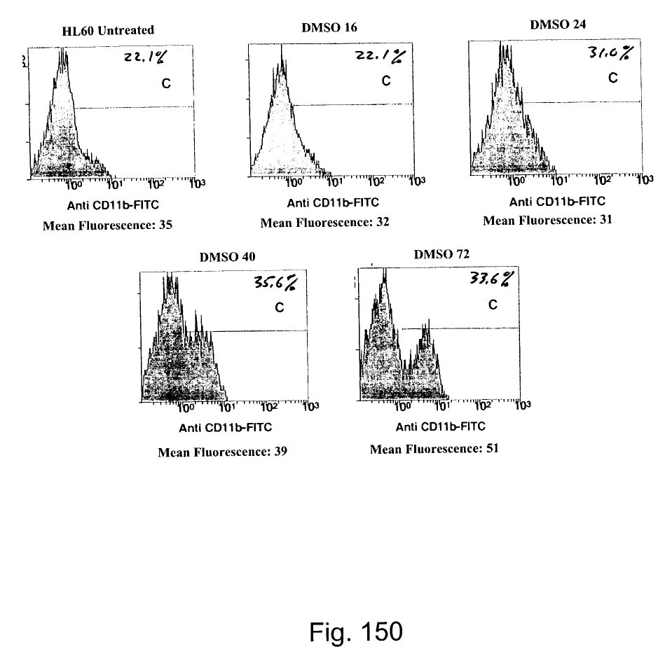

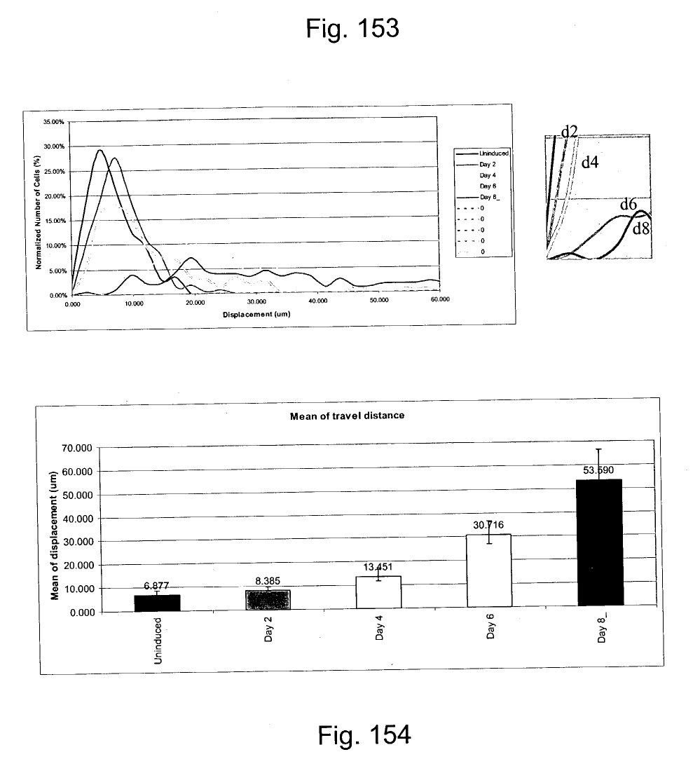

FIG. 153 illustrates a histogram of the displacement of the 3T3-L1 cells at day 2, day 4, day 6, and day 8 post-induction. Also shown in FIG. 153 are the uninduced control cells. FIG. 154 illustrates the mean travel distances of the uninduced control cells as well as the induced cells at day 2, day 4, day 6, and day 8 post-induction. An increase in mean travel distance is seen as early as two days after induction. As seen in FIG. 154, the mean travel distance progressively increases as more time elapsed. FIG. 155 shows the relative shift in mean travel distance over the eight period post-induction.

Secondary assays were conducted on induced 3T3-L1 cells over the same eight day period post-induction. One assay tested lipid accumulation using cells stained with BODIPY 505/515. FIG. 156 illustrates a graph of the fluorescent level as a function of days post-induction. As seen in FIG. 156, an increase in fluorescence is seen four days post-induction. FIG. 157 illustrates a comparison of the relative signal between BODIPY 505/515 and Optophoretic Analysis.

In yet another assay, commitment markers were measured over the eight day period post-induction. In this assay, mRNA coding for the nuclear hormone receptor peroxisome proliferator-activated receptor γ (PPARγ) and mRNA coding for the CCAAT/enhancer binding protein C/EBPα were measured. PPARγ and C/EBPα are critical transcription factors in adipogenesis It is known that after induction of adipocytes, levels of PPARγ and C/EBPα increase and induce gene expression changes of mature adipocytes. FIG. 158 illustrates normalized levels of PPARγ and C/EBPα mRNA (ratio target gene/Ribosomal 18S) over the eight day period post-induction.

Additional assays were performed for multilocular adipocyte specific products. In one assay, levels of mRNA coding for the protein Leptin were measured at days 2, 4, 6, and 8 post-induction. Leptin is a protein that plays an important role in how the body manages its supply of fat. In another assay, levels of mRNA coding for the adipocyte fatty acid binding protein aP2 were measured. FIG. 159 illustrates the normalized levels of Leptin mRNA at days 2, 4, 6, and 8 post-induction (as well as the uninduced control). Similarly, FIG. 160 illustrates the normalized levels of aP2 mRNA at days 2, 4, 6, and 8 post-induction (as well as the uninduced control).

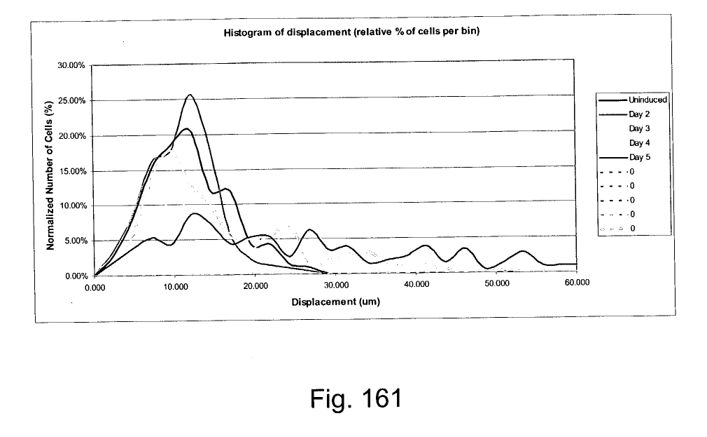

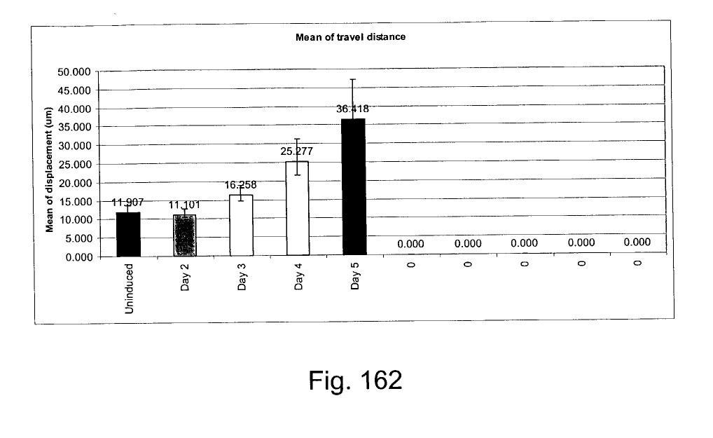

In still another experiment, induction of 3T3-L1 cells were monitored over a five day time period with measurements taken at day 2, day 3, day 4, and day 5 post-induction. The 3T3-L1 cells were prepared and harvested using the same methods described above with respect to the eight day Optophoretic monitoring of adipogenesis. FIG. 161 illustrates a histogram of the displacement of the 3T3-L1 cells at day 2, day 3, and day 5 post-induction. Also shown in FIG. 161 are the uninduced control cells. FIG. 162 illustrates the mean travel distances of the uninduced control cells as well as the induced cells at day 2, day 3, and day 5 post-induction. An increase in mean travel distance is seen as early as three days after induction. As seen in FIG. 162, the mean travel distance progressively increases as more time elapsed.

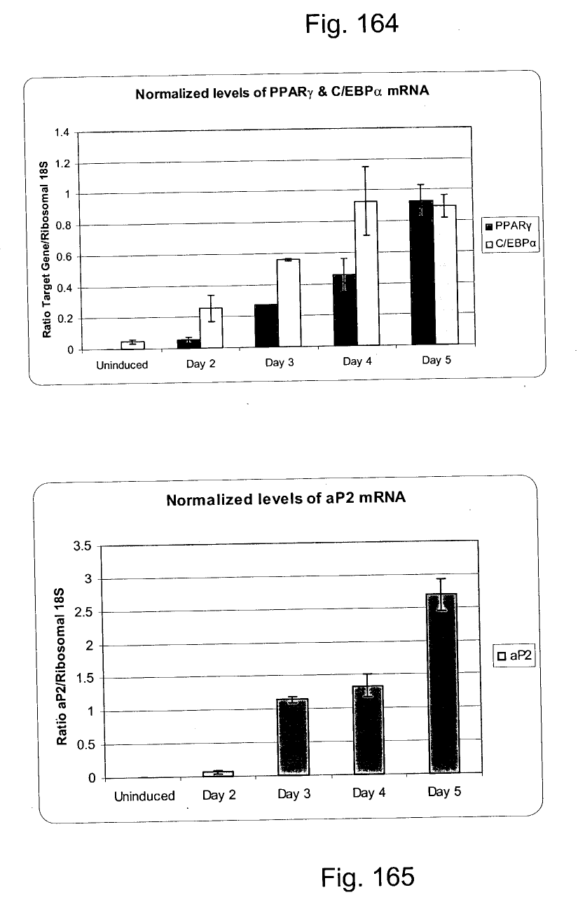

Secondary assays were performed over the same five day induction time period. Lipid accumulation was tested using cells stained with BODIPY 505/515. FIG. 163 illustrates a graph of the fluorescent level as a function of days post-induction. In yet another secondary assay, commitment markers were measured over the five day period post-induction. In this assay, mRNA coding for PPARγ and mRNA coding for C/EBPα were measured. FIG. 164 illustrates normalized levels of PPARγ and C/EBPα mRNA (ratio target gene/Ribosomal 18S) over the five day period post-induction. In still another assay, levels of mRNA coding for the adipocyte fatty acid binding protein aP2 were measured. FIG. 165 illustrates the normalized levels of aP2 mRNA at days 2, 3, 4, and 5 post-induction (as well as the uninduced control).

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

What is claimed is:

1. A method for detecting cellular differentiation using an optical gradient comprising the steps of:
(a) providing a plurality of cells;
(b) moving the optical gradient relative to the plurality of cells so as to cause displacement of at least some of the plurality of cells;
(c) measuring the travel distance of at least some of the plurality of cells;
(d) repeating steps (b) and (c) a plurality of times; and
(e) identifying those cells having changing travel distances.

2. The method according to claim 1, wherein the identified cells have travel distances that increase over time.

3. The method according to claim 1, wherein the identified cells have travel distances that decrease over time.

4. The method according to claim 1, wherein the cells comprise HL-60 cells.

5. The method according to claim 1, further comprising the step of exposing the cells to a chemical compound.

6. The method according to claim 1, wherein a change in travel distance is detected at least as early as 16 hours after testing.

7. A method of detecting adipogenesis using an optical gradient comprising the steps of:
(a) providing a plurality of preadipocytes;
(b) moving the optical gradient relative to the plurality of preadipocytes so as to cause displacement of at least some of the plurality of preadipocytes;
(c) measuring the travel distance of at least some of the plurality of preadipocytes;
(d) repeating steps (b) and (c) a plurality of times; and
(e) identifying those preadipocytes having increased travel distances.

8. The method of claim 7, wherein a preadipocytes having increased travel distances are detected at least as early as 2 days after testing.

9. A method of monitoring adipogenesis using an optical gradient comprising the steps of:
(a) providing a plurality of cells comprising preadipocytes;
(b) moving the optical gradient relative to the plurality of cells so as to cause displacement of at least some of the plurality of cells;
(c) measuring the travel distance of at least some of the plurality of cells;
(d) repeating steps (b) and (c) a plurality of times; and
(e) monitoring those cells exhibiting increased travel distances over time.
